(19) United States
(12) Patent Application Publication
(10) Pub. No.: US 2011/0118340 A1
(43) Pub. Date: May 19, 2011

(54) DELIVERY OF RNAI CONSTRUCTS TO OLIGODENDROCYTES

(76) Inventors: Muthiah Manoharan, Cambridge, MA (US); Kallanthottathil G. Rajeev, Cambridge, MA (US); Dinah Sah, Cambridge, MA (US); William Querbes, Cambridge, MA (US); Pamela Tan, Kulmbach (DE); Qingmin Chen, Cambridge, MA (US)

(21) Appl. No.: 12/866,444

(22) PCT Filed: Feb. 6, 2009
(86) PCT No.: PCT/US09/33420

§ 371 Date: Jan. 24, 2011

Related U.S. Application Data
(60) Provisional application No. 61/027,340, filed on Feb. 8, 2008.
(60) Provisional application No. 61/033,910, filed on Mar. 5, 2008.
(60) Provisional application No. 61/039,069, filed on Mar. 24, 2008.
(60) Provisional application No. 61/085,683, filed on Aug. 1, 2008.
(60) Provisional application No. 61/105,376, filed on Oct. 14, 2008.

(51) Int. Cl. A61K 31/7088; A61P 31/12; A61P 25/00; A61P 25/18; A61P 25/16; A61P 25/28

(52) U.S. Cl. 514/44 R

(57) ABSTRACT

The invention provides methods for delivering a double-stranded nbonucleic acid (dsRNA) to the central nervous system of a subject, and particularly, to oligodendrocytes of a subject by localized delivery to the brain, e.g., to the corpus caïlosum. For example, the dsRNA molecules can include a first sequence that is selected from the Sroup consisting of the sense sequences of Tables 8, 10, 13-16, and a second sequence selected from the group consisting of the antisense sequences of Tables 8, 10, and 13-16. The dsRNA molecules can include naturally occurring nucleotides or can include at least one modified nucleotide, such as a 2′-O-methyl modified nucleotide, a nucleotide comprising a 5′-phosphorothioate group, or a terminal nucleotide linked to a conjugate group, such as to a cholesteryl derivative or a vitamin E group. Alternatively, the modified nucleotide may be chosen from the group consisting of a 2f-deoxy-2′-fliιioro modified nucleotide, a 2′-de-oxy-modified nucleotide, a locked nucleotide, an abasic nucleotide, 2′-amino-modified nucleotide, 2′-alkyl-modified nucleotide, morpholino nucleotide, a phosphoramidate, and a non-natural bas comprising nucleotide. Generally, such modified sequences will be based on a first sequence of a dsRNA selected from the group consisting of the sense sequences of Tables 8, 10, and 13-16, and a second sequence selected from the group consisting of the antisense sequences of Tables 8 10, and 13-16.

### CROSS-REFERENCE TO RELATED APPLICATIONS

This application claims priority to U.S. Application Ser. No. 61/027,340, filed Feb. 8, 2008; to U.S. Ser. No. 61/033,910, filed Mar. 5, 2008; to U.S. Ser. No. 61/039,069, filed Mar. 24, 2008; to U.S. Ser. No. 61/085,683, filed Aug. 1, 2008; and to U.S. Ser. No. 61/105,376, filed Oct. 14, 2008. The entire contents of all of these provisional applications are hereby incorporated by reference in the present application.

### FIELD OF THE INVENTION

This invention relates to methods of delivering an siRNA to the central nervous system of a subject by localized delivery to oligodendrocytes.

### BACKGROUND OF THE INVENTION

Progressive multifocal leukoencephalopathy (PML) is a fatal demyelinating disease of the central nervous system which results from reactivation of the latent polyomavirus JC virus (JCV) and its productive replication in glial cells of the human brain (Berger, J. R. (1995) J. Neurovirol. 1:5-18). Once a rare disease primarily seen in patients with impaired immune systems due to lymphoproliferative and myeloproliferative disorders, PML has become one of the major neurologic problems among patients with AIDS (Cinque, P., (2003). J. Neurovirol. 9 (Suppl. 1):88-92).

It has been reported that between 4 and 8% of AIDS patients exhibit signs of PML, and JCV has been detected in the cerebrospinal fluid of affected patients, suggesting that there is active replication of the virus in the brain (Berger, J. R. (1995) J. Neurovirol. 1:5-18, Clifford, D. B., (2001) J. Neurovirol. 4:279). In addition, PML has recently been seen in patients undergoing experimental treatment with Tysbari™, an anti-VLA4 antobody, in combination with interferon. The histological hallmarks of PML include multifocal demyelinated lesions with enlarged eosinophilic nuclei in oligodendrocytes and enlarged bizarre astrocytes with lobulated hyperchromatic nuclei within white matter tracts of the brain (Cinque, P., (2003). J. Neurovirol. 9 (Suppl. 1):88-92), although in some instances atypical features that include a unifocal pattern of demyelination and involvement of the gray matter have been reported (Sweeney, B. J., (1994). J. Neurol. Neurosurg. Psychiatry 57:994-997). Earlier observations from in vitro cell culture studies and an in vivo evaluation of JCV in clinical samples led to early assumptions that oligodendrocytes and astrocytes are the only cells that support productive viral infections (Gordon, J. (1998) Int. J. Mol. Med. 1:647-655). Accordingly, molecular studies have provided evidence for cell-type-specific transcription of the viral early genome in cells derived from the central nervous system (Raj, G. V., (1995) Virology 10:283-291). However, subsequent studies have shown low, but detectable, levels of JCV gene expression in normeural cells, including B cells, and noticeably high levels of production of the viral early protein in several neural and normeural tumor cells in humans (Gordon, J. (1998) Int. J. Mol. Med. 1:647-655, Khalili, K., 2003. Oncogene 22:5181-5191).

Like the other polyomaviruses, JCV is a small DNA virus whose genome can be divided into three regions that encompass the transcription control region; the genes responsible for the expression of the viral early protein, T antigen; and the genes encoding the viral late proteins, VP1, VP2, and VP3. In addition, the late genome is also responsible for production of an auxiliary viral protein, agnoprotein. T-antigen expression is pivotal for initiation of the viral lytic cycle, as this protein stimulates transcription of the late genes and induces the process of viral DNA replication. Recent studies have ascribed an important role for agnoprotein in the transcription and replication of JCV, as inhibition of its production significantly reduced viral gene expression and replication (M. Safak et al., unpublished observations). Furthermore, the agnoprotein dysregulates the cell cycle by altering the expression of several cyclins and their associated kinases (Darbinyan, A., (2002) Oncogene 21:5574-5581).

Thus far, there are no effective therapies for the suppression of JCV replication and the treatment of PML. Cytosine arabinoside (AraC) has been tested for the treatment of PML patients, and the outcome in some instances revealed a remission of JCV-associated demyelination (Aksamit, A. (2001) J. Neurovirol. 7:386-390). Reports from the AIDS Clinical Trial Group Organized Trial 243, however, have suggested that there is no difference in the survival of human immunodeficiency virus type 1 (HIV-1)-infected patients with PML and that of the control population, although in other reports it has been suggested that the failure of AraC in the AIDS Clinical Trial Group trial may have been due to insufficient delivery of the AraC via the intravenous and intrathecal routes (Levy, R. M., (2001) J. Neurovirol. 7:382-385). Based on in vitro studies showing the ability of inhibitors of topoisomerase to suppress JCV DNA replication, the topoisomerase inhibitor topotecan was used for the treatment of AIDS-PML patients, and the results suggested that topotecan treatment may be associated with a decreased lesion size and prolonged survival (Royal, W., III, (2003) J. Neurovirol. 9:411-419).

Double-stranded RNA molecules (dsRNA) have been shown to block gene expression in a highly conserved regulatory mechanism known as RNA interference (RNAi). WO 99/32619 (Fire et al.) discloses the use of a dsRNA of at least 25 nucleotides in length to inhibit the expression of genes in C. elegans. dsRNA has also been shown to degrade target RNA in other organisms, including plants (see, e.g., WO 99/53050, Waterhouse et al.; and WO 99/61631, Heifetz et al.), Drosophila (see, e.g., Yang, D., et al., Curr. Biol. (2000) 10:1191-1200), and mammals (see WO 00/44895, Limmer; and DE 101 00 586.5, Kreutzer et al.). This natural mechanism has now become the focus for the development of a new class of pharmaceutical agents for treating disorders that are caused by the aberrant or unwanted regulation of a gene.

### SUMMARY OF THE INVENTION

The invention provides methods for delivering a double-stranded ribonucleic acid (dsRNA) to the central nervous system of a subject, and particularly, to oligodendrocytes of a subject by localized delivery to the brain, e.g., to the corpus callosum.

In one aspect, the invention provides, a method for delivering a nucleic acid drug, e.g., a dsRNA, to a subject, e.g., to the oligodendrocytes of a subject. The subject can be a mammal, such as a human or non-human primate. Delivery can be, for example, by localized delivery, e.g., injection or infusion, into the brain, such as into white matter of the brain, e.g., into the corpus callosum. In another embodiment, the dsRNA is delivered by intrastriatal infusion, into the striatum, such as into the corpus striatum. In one embodiment, delivery to oligodendrocytes is by infusion to the corpus callosum.

In one embodiment, the nucleic acid drug is a double-stranded ribonucleic acid (dsRNA) molecule for inhibiting the expression of one of the genes of the JC virus and for inhibiting viral replication. The dsRNA can include at least two sequences that are complementary to each other. The dsRNA can include a sense strand comprising a first sequence and an antisense strand comprising a second sequence. The antisense strand comprises a nucleotide sequence which is substantially complementary to at least part of an mRNA encoded by a gene from the JC Virus, and the region of complementarity is typically less than 30 nucleotides in length, generally 19-24 nucleotides in length. In one embodiment, the dsRNA, when evaluated in an in vitro assay described herein, inhibits expression of a gene from the JC Virus by at least 40%.

For example, the dsRNA molecules can include a first sequence that is selected from the group consisting of the sense sequences of Tables 8, 10, 13-16, and a second sequence selected from the group consisting of the antisense sequences of Tables 8, 10, and 13-16. The dsRNA molecules can include naturally occurring nucleotides or can include at least one modified nucleotide, such as a 2′-O-methyl modified nucleotide, a nucleotide comprising a 5′-phosphorothioate group, or a terminal nucleotide linked to a conjugate group, such as to a cholesteryl derivative or a vitamin E group. Alternatively, the modified nucleotide may be chosen from the group consisting of a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. Generally, such modified sequences will be based on a first sequence of a dsRNA selected from the group consisting of the sense sequences of Tables 8, 10, and 13-16, and a second sequence selected from the group consisting of the antisense sequences of Tables 8, 10, and 13-16.

In one embodiment, the dsRNA targets VP1, T antigen, VP2 or VP3. In another embodiment, only a single gene, e.g., VP1, is targeted. In yet another embodiment, the T Antigen gene is targeted. In yet another embodiment, more than one type of dsRNA is administered, but only one gene is targeted.

In one embodiment, the dsRNA does not activate the immune system, e.g., it does not increase cytokine levels, such as TNF-alpha or IFN-alpha levels. For example, when measured by an assay, such as an in vitro PBMC assay, such as described herein, the increase in levels of TNF-alpha or IFN-alpha, is less than 30%, 20%, or 10% of control cells treated with a control dsRNA, such as a dsRNA that does not target a JC Virus gene.

In another aspect, the invention provides a method for inhibiting the expression of a JC virus gene in a subject. The subject can be a mammal, such as a human or non-human primate. The method includes delivery, e.g., localized delivery, such as by injection or infusion, of a nucleic acid drug, such as a dsRNA, into a region of the brain, such as into the corpus callosum. In one embodiment, the method provides delivery to oligodendrocytes by infusion into the corpus callosum.

In one embodiment, the invention provides oligonucleotides and method for silencing CNPase mRNA in a subject. The subject can be a mammal, such as a human or non-human primate. The method includes delivery, e.g., localized delivery, such as by injection, infusion or intraparenchymal convection enhanced delivery (CED) of a nucleic acid drug, such as a dsRNA, into a region of the brain, such as into the corpus callosum. In one embodiment, the method provides delivery to oligodendrocytes by infusion into the corpus callosum.

In one embodiment, the nucleic acid drug is a double-stranded ribonucleic acid (dsRNA) molecule for inhibiting the expression of one of the genes of the JC virus and for inhibiting viral replication. The dsRNA can include at least two sequences that are complementary to each other. The dsRNA can include a sense strand having a first sequence and an antisense strand having a second sequence. The antisense strand includes a nucleotide sequence that is substantially complementary to at least part of an mRNA encoded by a gene from the JC Virus, and the region of complementarity is less than 30 nucleotides in length, generally 19-24 nucleotides, e.g., 19 to 21 nucleotides in length. In some embodiments, the dsRNA is from about 10 to about 15 nucleotides, and in other embodiments the dsRNA is from about 25 to about 30 nucleotides in length. In a one embodiment, the dsRNA, when evaluated in an in vitro assay described herein, inhibits expression of a gene from the JC Virus by at least 40%.

For example, the dsRNA molecules can include a first sequence selected from the group consisting of the sense sequences of Tables 8, 10, and 13-16, and a second sequence selected from the group consisting of the antisense sequences of Tables 8, 10, and 13-16. The dsRNA molecules can include naturally occurring nucleotides and can also include at least one modified nucleotide, such as a 2′-O-methyl modified nucleotide, a nucleotide comprising a 5′-phosphorothioate group, or a terminal nucleotide linked to conjugate group, such as to a cholesteryl derivative or vitamin E group. Alternatively, the modified nucleotide may be chosen from the group consisting of a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. Generally, such modified sequences of a dsRNA will be based on a first sequence selected from the group consisting of the sense sequences of Tables 8, 10, and 13-16, and a second sequence selected from the group consisting of the antisense sequences of Tables 8, 10, and 13-16.

In one embodiment, the dsRNA targets VP1, T antigen, VP2 or VP3. In another embodiment, only a single gene, e.g., VP1, is targeted. In yet another embodiment, the T Antigen gene is targeted. In yet another embodiment, more than one type of dsRNA is administered, but only one gene is targeted.

In one embodiment, the dsRNA does not activate the immune system, e.g., it does not increase cytokine levels, such as TNF-alpha or IFN-alpha levels. For example, when measured by an assay, such as an in vitro PBMC assay, such as described herein, the increase in levels of TNF-alpha or IFN-alpha, is less than 30%, 20%, or 10% of control cells treated with a control dsRNA, such as a dsRNA that does not target a JC Virus gene.

In another aspect, the invention provides a method for treating, preventing, delaying or managing a pathological process or symptom mediated by JC virus infection, such as PML, in a subject. The subject can be a mammal, such as a human or non-human primate. The method includes delivery, e.g., localized delivery, such as by injection or infusion, of a therapeutic amount of a nucleic acid drug, e.g., a dsRNA, into the white matter of the brain, such as into the corpus callosum of the subject. In one embodiment, the method provides delivery to oligodendrocytes by infusion into the corpus callosum (e.g., by intracallosal infusion). In another embodiment, the method treats, prevents or delays development of a brain tumor, such as a glioblastoma multiforme.

In one embodiment, the nucleic acid drug is a double-stranded ribonucleic acid (dsRNA) molecule for inhibiting the expression of one of the genes of the JC virus and for inhibiting viral replication. The dsRNA can include at least two sequences that are complementary to each other. The dsRNA can include a sense strand having a first sequence and an antisense strand having a second sequence. The antisense strand includes a nucleotide sequence that is substantially complementary to at least part of an mRNA encoded by a gene from the JC Virus, and the region of complementarity is typically less than 30 nucleotides in length, generally 19-24 nucleotides in length. In one embodiment, the dsRNA, when evaluated in an in vitro assay described herein, inhibits expression of a gene from the JC Virus by at least 40%.

For example, the dsRNA molecule can include a first sequence selected from the group consisting of the sense sequences of Tables 8, 10, and 13-16, and a second sequence selected from the group consisting of the antisense sequences of Tables 8, 10, and 13-16. The dsRNA molecules can include naturally occurring nucleotides and can also include at least one modified nucleotide, such as a 2′-O-methyl modified nucleotide, a nucleotide comprising a 5′-phosphorothioate group, or a terminal nucleotide linked to a conjugate group, such as to a cholesteryl derivative or a vitamin E group. Alternatively, the modified nucleotide may be chosen from the group consisting of a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. Generally, such modified sequences of a dsRNA will be based on a first sequence selected from the group consisting of the sense sequences of Tables 8, 10, and 13-16, and a second sequence selected from the group consisting of the antisense sequences of Tables 8, 10, and 13-16.

In one embodiment, the dsRNA targets VP1, T antigen, VP2 or VP3. In another embodiment, only a single gene, e.g., VP1, is targeted. In yet another embodiment, the T Antigen gene is targeted. In yet another embodiment, more than one type of dsRNA is administered, but only one gene is targeted.

In another embodiment, the dsRNA does not activate the immune system, e.g., it does not increase cytokine levels, such as TNF-alpha or IFN-alpha levels. For example, when measured by an assay, such as an in vitro PBMC assay, such as described herein, the increase in levels of TNF-alpha or IFN-alpha, is less than 30%, 20%, or 10% of control cells treated with a control dsRNA, such as a dsRNA that does not target a JC Virus gene.

In one aspect, the invention provides a pharmaceutical composition for inhibiting the replication of the JC virus in an organism, generally a human subject, containing one or more of a dsRNA featured in the invention and a pharmaceutically acceptable carrier or delivery vehicle and disposed in a device configured to provide localized delivery to the brain, such as into the white matter of the brain, e.g., into the corpus callosum. Delivery can further be administered directly into oligodendrocytes, such as into oligodendrocytes of the corpus callosum. Delivery can also be into the striatum, such as by intrastriatal infusion.

In another embodiment, the invention provides vectors for inhibiting the expression of a gene of the JC virus in a cell, where the vector includes a regulatory sequence operably linked to a nucleotide sequence that encodes at least one strand of one of the dsRNAs featured in the invention.

In another embodiment, the invention provides a cell containing a vector for inhibiting the expression of a gene of the JC virus in a cell. The vector includes a regulatory sequence operably linked to a nucleotide sequence that encodes at least one strand of one of the dsRNAs featured in the invention.

In another aspect, the invention provides a method for delivering a double-stranded ribonucleic acid (dsRNA) to the central nervous system of a subject, and particularly, to oligodendrocytes of a subject by localized delivery to the brain, e.g., to the corpus callosum.

In another aspect, the invention provides a method for delivering a dsRNA by localized delivery in to the corpus callosum of the subject, such as to an oligodendrocyte of the subject, where the dsRNA targets a CNPase nucleic acid. For example, the dsRNA targeting CNPase RNA can include a first sequence selected from the group consisting of the sense sequences of Tables 1, and 17-19, and a second sequence selected from the group consisting of the antisense sequences of Tables 1, and 17-19. In one embodiment, the dsRNA targeting CNPase is AD3222. In yet another aspect, the invention provides a method for treating, preventing or managing a neurological disorder mediated by CNPase in a subject, such as by delivering a dsRNA by localized delivery into the corpus callosum of the subject, such as into an oligodendrocyte of the subject. In certain embodiments, the CNPase RNA includes a first sequence selected from the group consisting of the sense sequences of Tables 1, and 17-19, and a second sequence selected from the group consisting of the antisense sequences of Tables 1, and 17-19. In one embodiment, the dsRNA targeting CNPase is AD3222. In other embodiments, the neurological disorder is schizophrenia or Down's Syndrome.

In another aspect, the invention provides a method for decreasing CNPase mRNA levels in a subject by, for example, delivering a dsRNA by localized delivery into the corpus callosum of the subject. In certain embodiments, the CNPase RNA includes a first sequence selected from the group consisting of the sense sequences of Tables 1, and 17-19, and a second sequence selected from the group consisting of the antisense sequences of Tables 1, and 17-19. In one embodiment, the dsRNA targeting CNPase is AD3222.

In yet another aspect, the invention features a method of treating a neurodegenerative disease in a subject by delivering a dsRNA by localized delivery into the central nervous system, such as into the corpus callosum (e.g., into an oligodendrocyte of the corpus callosum). In one embodiment the dsRNA targets a gene endogenous to the subject, and delivery of the dsRNA is for the treatment of, e.g., Parkinson's disease, multiple sclerosis, amyotrophic lateral sclerosis, autoimmune encephalomyelitis, Alzheimer's disease, stroke or Huntington's disease.

The details of one or more embodiments featured in the invention are set forth in the accompanying drawings and the description below. Other features, objects, and advantages featured in the invention will be apparent from the description and drawings, and from the claims.

### BRIEF DESCRIPTION OF THE FIGURES

FIGS. 1A, 1B, and 1C show silencing of CNP mRNA in vivo as measured by branched DNA analysis following intraparenchymal infusion of CNP siRNA AD-12436 into the corpus callosum of rats. FIG. 1A is a bar graph showing the specificity of AD-12436 against CNP mRNA, as compared to negative controls PBS and dsRNA AD-1955, which targets luciferase.

FIG. 1B is a bar graph showing the dose dependence of the gene-silencing effect of dsRNA on CNP RNA levels. FIG. 1C is a bar graph showing the sustained effected of dsRNA on RNA inhibition for up to seven days.

FIG. 2 illustrates cleavage of CNP mRNA in vivo as mediated by CNP siRNA AD-12436. The cleavage site was detected by 5′ RACE.

FIG. 3A is a graph showing silencing of rat CNPase. Data points marked with asterisks are statistically significant compared with PBS-treated animals (*** P<0.001, ** P<0.01; ANOVA). FIG. 3B is a graph showing silencing of primate CNPase.

FIG. 4 is a graph showing that VP1 siRNAs are more effective inhibitors when alone, than they are in combination with siTAg siRNAs.

FIGS. 5A and 5B are graphs showing that JCV siRNAs do not produce unwanted cytokine responses, as IFN-alpha (FIG. 5A) and TNF-alpha (FIG. 5B) levels remained low after contact with the siRNAs.

FIGS. 6A, 6B and 6C show silencing of CNP mRNA in vivo as measured by branched DNA analysis following intraparenchymal convection enhanced delivery (CED) of CNP siRNA AD-3222 and AD-3178 into the corpus callosum of rats. FIG. 6A is a bar graph comparing the gene-silencing effect of CNP siRNA AD-3222 and AD-3178. FIG. 6B is a bar graph showing the dose dependence of the gene-silencing effect of AD-3222 on CNP RNA levels. FIG. 6C is a bar graph showing the sustained effected of AD-3222 on RNA inhibition for up to seven days. FIG. 6D is a bar graph comparing the gene-silencing effect of CNP siRNA AD-3222 and AD-12436 with intrastriatal or intracortical CED infusion. Data points marked with asterisks are statistically significant compared with PBS-treated animals (*** P<0.001, ** P<0.01, * P<0.05; PBS Two-way ANOVA).

FIGS. 7A and 7B show silencing of CNP mRNA in vivo as measured by branched DNA analysis following intraparenchymal convection enhanced delivery (CED) of CNP siRNA AD-3181 and AD-3569 into the corpus callosum of rats. FIG. 7A is bar graph showing the dose dependence of the gene-silencing effect of AD-3181 on CNP RNA levels. FIG. 7B is a bar graph comparing the gene-silencing effect of CNP siRNA AD-3181 and AD-3569. FIG. 7C is a bar graph comparing the gene-silencing effect of CNP siRNA AD-3569 with dicer CNP siRNA AD-18233. Data points marked with asterisks are statistically significant compared with PBS-treated animals (*** P<0.001, ** P<0.01, * P<0.05; PBS Two-way ANOVA).

FIG. 8 is a bar graph comparing the gene-silencing effect of CNP siRNA AD-3569 and AD-18528.

FIGS. 9A and 9B are the complete genome sequence of JC virus as found at GenBank Accession No. NC_{—}001699 (GenBank version dated Dec. 24, 2007).

FIGS. 10A and 10B are the sequence of the human CNPase mRNA transcript as reported at GenBank Accession No. NM_{—}033133 (GenBank version dated Jan. 13, 2008).

### DETAILED DESCRIPTION OF THE INVENTION

The invention provides methods of administering double-stranded ribonucleic acid (dsRNA) into a cell of the central nervous system (CNS), such as an oligodendrocyte, for inhibiting the expression of a gene in the central nervous system of a mammal by localized delivery to the brain, such as to the corpus callosum or into other areas of white matter. The dsRNA can target, e.g., an endogenous gene, such as the CNPase gene, or a gene from a pathogen, such as a virus, e.g., the JC Virus. The invention also provides compositions and methods for treating pathological conditions and diseases in a mammal caused by JC virus infection using dsRNAs. A dsRNA directs the sequence-specific degradation of mRNA through a process known as RNA interference (RNAi).

The dsRNA suitable for use in the methods described herein include an RNA strand (the antisense strand) having a region that is less than 30 nucleotides in length, generally 19-24 nucleotides in length, and that is substantially complementary to at least part of an mRNA transcript of a gene from the JC Virus. The use of these dsRNAs enables the targeted degradation of mRNAs of genes that are implicated in replication and or maintenance of JC virus infection and the occurance of PML in a subject infected with the JC virus. Using cell-based and animal assays, the present inventors have demonstrated that very low dosages of these dsRNA can specifically and efficiently mediate RNAi, resulting in significant inhibition of expression of a gene from the JC Virus. Thus, the methods and compositions featured herein include dsRNAs useful for treating pathological processes mediated by JC viral infection, e.g. cancer, by targeting a gene involved in JC virus relication and/or maintainance in a cell.

The following detailed description discloses how to make and use the dsRNA and compositions containing dsRNA to inhibit the expression of a gene from the JC virus, as well as compositions and methods for treating diseases and disorders caused by the infection with the JC virus, such as PML. The pharmaceutical compositions suitable for use in the featured methods include a dsRNA having an antisense strand with a region of complementarity that is less than 30 nucleotides in length, generally 19-24 nucleotides in length, and is substantially complementary to at least part of an RNA transcript of a gene from the JC Virus, together with a pharmaceutically acceptable carrier.

Accordingly, in certain aspects, pharmaceutical compositions containing the dsRNAs described herein together with a pharmaceutically acceptable carrier, methods of using the compositions to inhibit expression of a gene from the JC Virus, and methods of using the pharmaceutical compositions to treat diseases caused by infection with the JC virus are provided.

In one embodiment, the invention provides oligonucleotides and methods for silencing CNPase mRNA in a subject, comprising delivering a dsRNA by localized delivery into the corpus callosum of the subject, wherein said dsRNA selected from the group consisting of duplex numbers AD3178, AD3222, AD12436, AD3181 and AD3569. The subject can be a mammal, such as a human or non-human primate. The method includes delivery, e.g., localized delivery, such as by injection, infusion or intraparenchymal convection enhanced delivery (CED) of a nucleic acid drug, such as a dsRNA, into a region of the brain, such as into the corpus callosum. In one embodiment, the method provides delivery to oligodendrocytes by infusion into the corpus callosum.

In one embodiment, a dsRNA delivered directly to a cell in the brain, such as to an oligodendrocyte in the brain, inhibits expression of a huntingtin gene, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21 (WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, mutations in the p53 tumor suppressor gene, mutations in the p53 family member DN-p63, mutations in the pRb tumor suppressor gene, mutations in the APC1 tumor suppressor gene, mutations in the BRCA1 tumor suppressor gene, mutations in the PTEN tumor suppressor gene, mLL fusion gene, BCR/ABL fusion gene, TEL/AML1 fusion gene, EWS/FLI1 fusion gene, TLS/FUS1 fusion gene, PAX3/FKHR fusion gene, AML1/ETO fusion gene, alpha v-integrin gene, Flt-1 receptor gene, tubulin gene, Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, 1-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCAT gene, SCA8 gene, allele gene found in LOH cells, or one allele gene of a polymorphic gene.

According to one aspect, the invention provides a method of treating a neurodegenerative disorder. As used herein, the phrase “neurodegenerative disorder” refers to any disorder, disease or condition of the nervous system (such as the CNS) which is characterized by gradual and progressive loss of neural tissue, neurotransmitter, or neural functions. Examples of neurodegenerative disorder include Parkinson's disease, multiple sclerosis, amyotrophic lateral sclerosis, autoimmune encephalomyelitis, Alzheimer's disease, stroke and Huntington's disease.

### I. DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

As used herein, localized delivery to the corpus callosum, refers to local delivery by direct introduction of the drug into oligodendrocytes, such as oligodendrocytes of the corpus callosum. Localized delivery includes by injection or infusion. Localized delivery excludes systemic administration.

“G,” “C,” “A,” “T,” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, thymidine and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences having such replacement moieties are embodiments featured in the invention.

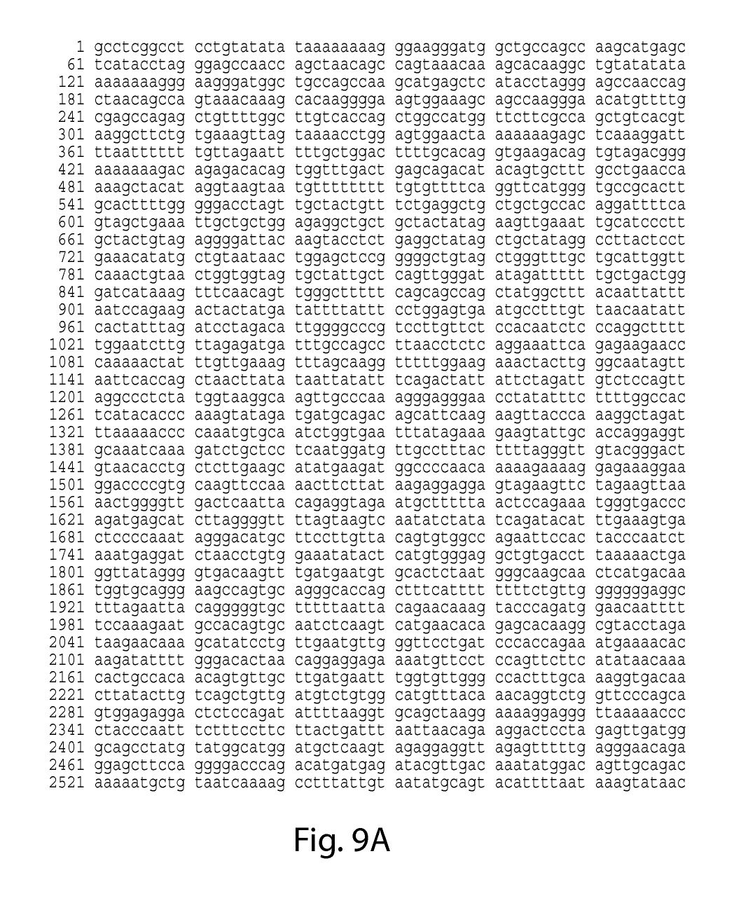

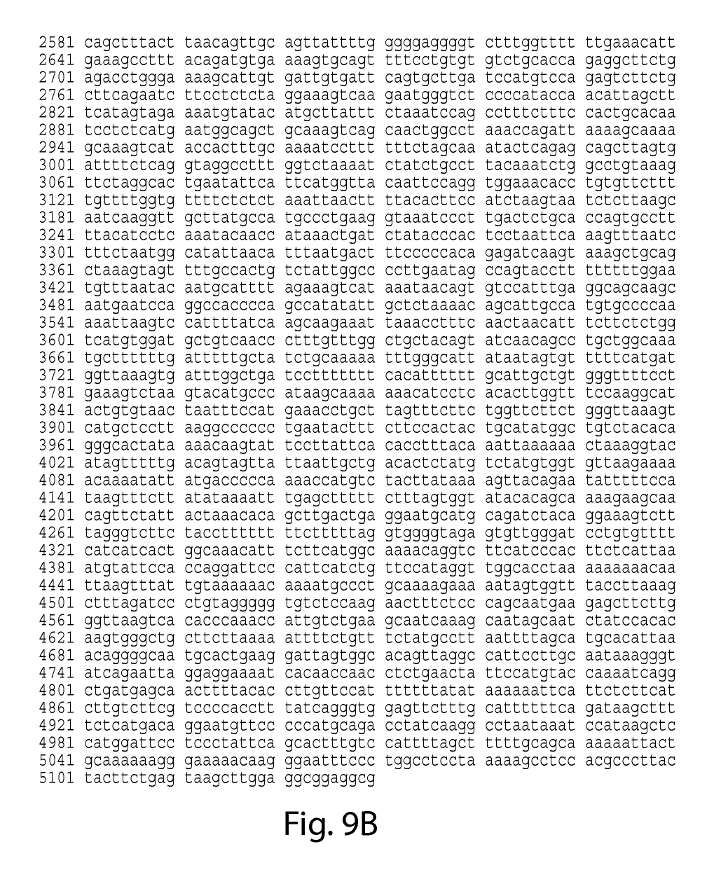

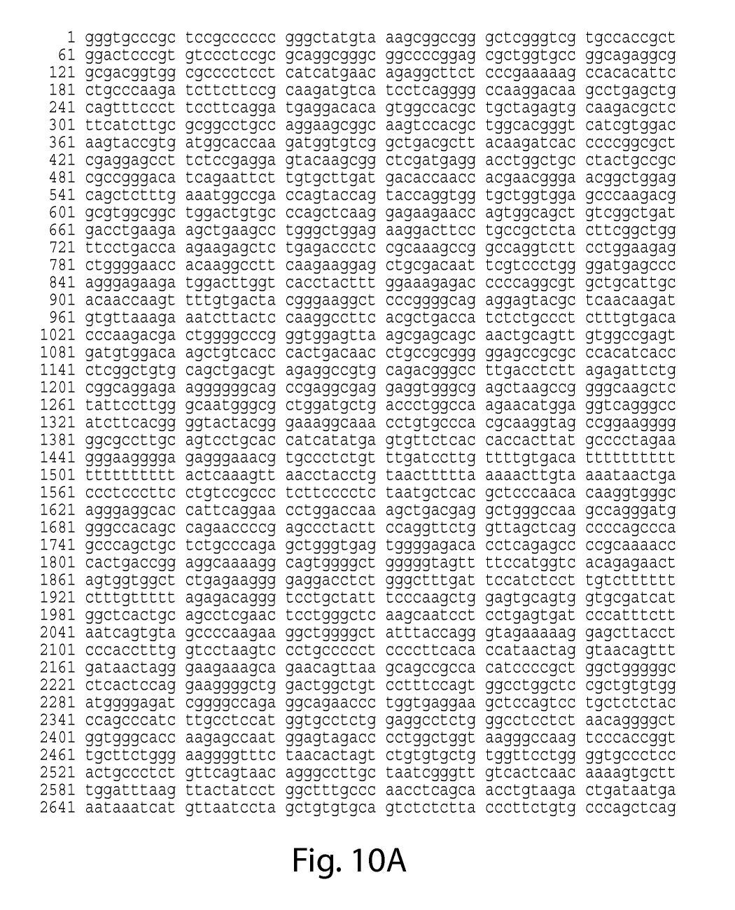

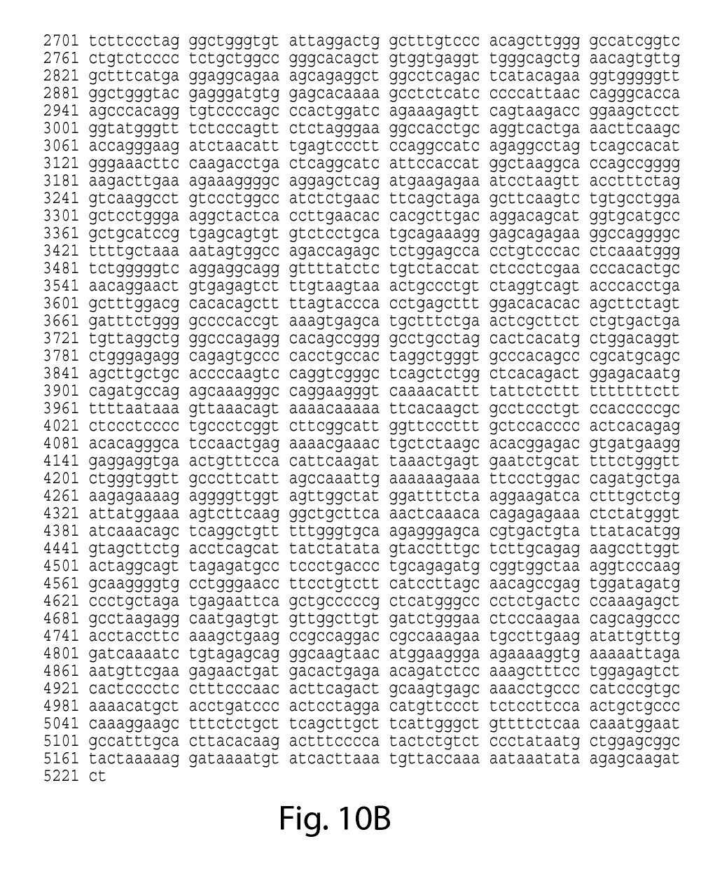

As used herein, “JC virus” refers to the latent polyomavirus JC Virus that has a reference sequence at GenBank Accession No. NC_{—}001699 (GenBank version dated Dec. 24, 2007) (see also FIGS. 9A and 9B). JC Virus is also known as JC polyomavirus. Other accession numbers of various JCVirus sequences include AB038249.1-AB038255.1, AB048545.1-AB048582.1, AB074575.1-AB074591.1, AB077855.1-AB077879.1, AB081005.1-AB081030.1, AB081600.1-AB081618.1, AB081654.1, AB092578.1-AB092587.1, AB103387.1, AB103402.1-AB103423.1, AB104487.1, AB113118.1-AB113145.1, AB118651.1-AB118659.1, AB126981.1-AB127027.1, AB127342.1, AB127344.1, AB127346.1-AB127349.1, AB127352.1-AB127353.1, AB198940.1-AB198954.1, AB220939.1-AB220943.1, AF004349.1-AF004350.1, AF015526.1-AF015537.1, AF015684.1, AF030085.1, AF281599.1-AF281626.1, AF295731.1-AF295739.1, AF300945.1-AF300967.1, AF363830.1-AF363834.1, AF396422.1-AF396435.1, AY121907.1-AY121915.1, NC_{—}001699.1, U61771.1, U73500.1-U73502.1.

As used herein, “CNPase” refers to a gene in a cell. CNPase is also known as CNP; cyclic nucleotide phosphodiesterase; 2′,3′-cyclic nucleotide 3′ phosphodiesterase; CNP1; and 2′,3′ cyclic nucleotide 3′ phosphohydrolase. The sequence of a human CNPase mRNA transcript can be found at GenBank Accession No. NM_{—}033133 (GenBank version dated Jan. 13, 2008).

CNPase is an enzyme found mainly in the central nervous system of vertebrates. The enzyme is associated with myelin, including oligodendroglial plasma membrane and uncompacted myelin (myelin-like fraction), which are in contact with glial cytoplasm. CNPase is also firmly associated with tubulin from brain tissue. CNPase acts as a microtubule-associated protein in promoting microtubule assembly at higher mole ratios. CNPase catalyzes the hydrolysis of 2′,3′-cyclic nucleotides to produce 2′-nucleotides in vitro, and is a member of the 2H phosphoesterase family.

As used herein, “target sequence” refers to a contiguous portion of the nucleotide sequence of an mRNA molecule formed during the transcription of a gene from the JC Virus, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide havng the first nucleotide sequence to the oligonucleotide or polynucleotide having the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA having one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., encoding JC virus). For example, a polynucleotide is complementary to at least a part of a JC virus mRNA if the sequence is substantially complementary to a non-interrupted portion of a mRNA encoding JC virus.

The term “double-stranded RNA” or “dsRNA”, as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs. dsRNAs as used herein are also referred to as “siRNAs” (short interfering RNAs).

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA that includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches may be in the internal or terminal regions of the molecule. Generally, the most tolerated mismatches are in the terminal regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

“Introducing into a cell,” when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell,” where the cell is part of a living organism. In such instance, introduction into the cell will include the delivery to the organism. For example, for in vivo delivery, dsRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781. U.S. Pat. Nos. 5,032,401 and 5,607,677, and U.S. Publication No. 2005/0281781 are hereby incorporated by reference in their entirety. In vitro introduction into a cell includes methods known in the art such as electroporation and lipofection.

The terms “silence” and “inhibit the expression of,” “down-regulate the expression of,” “suppress the expression of,” and the like, in as far as they refer to a target gene, e.g., gene from JC Virus, herein refer to the at least partial suppression of the expression of a target gene, e.g., from the JC Virus, as manifested by a reduction of the amount of target mRNA, e.g., JC Virus mRNA, which may be isolated from a first cell or group of cells in which a target gene is transcribed and which has or have been treated such that the expression of a target gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to target gene expression, e.g., the amount of protein encoded by a gene from the JC Virus, or the number of cells displaying a certain phenotype, e.g infection with the JC Virus. In principle, target genome silencing may be determined in any cell expressing the target, either constitutively or by genomic engineering, and by any appropriate assay. However, when a reference is needed in order to determine whether a given dsRNA inhibits the expression of a target gene by a certain degree and therefore is encompassed by the instant invention, the assay provided in the Examples below and those known in the art shall serve as such reference. For example, in certain instances, expression of a gene from the JC Virus is suppressed by at least about 5%, 10%, 15%, 20%, 25%, 30%, 35%, 40%, 45%, or 50% by administration of the double-stranded oligonucleotide. In some embodiments, a gene from the JC Virus is suppressed by at least about 60%, 70%, or 80% by administration of the double-stranded oligonucleotide. In some embodiments, a gene from the JC Virus is suppressed by at least about 85%, 90%, or 95% by administration of the double-stranded oligonucleotide.

As used herein in the context of JC virus expression, the terms “treat,” “treatment,” and the like, refer to relief from or alleviation of pathological processes mediated by JC virus infection. In the context of the present invention insofar as it relates to any of the other conditions recited herein below (other than pathological processes mediated by JC virus expression), the terms “treat,” “treatment,” and the like mean to relieve or alleviate at least one symptom associated with such condition, or to slow or reverse the progression of such condition. For example, administration of a dsRNA targeting JC virus to an oligodendrocyte of human having PML, can reduce one or more symptoms of PML, such as relief from the extreme weakness, lack of coordination or difficulty speaking experienced by those infected with PML.

As used herein, the phrases “therapeutically effective amount” and “prophylactically effective amount” refer to an amount that provides a therapeutic benefit in the treatment, prevention, or management of pathological processes mediated by JC virus infection or an overt symptom of pathological processes mediated by JC virus expression. The specific amount that is therapeutically effective can be readily determined by an ordinary medical practitioner, and may vary depending on factors known in the art, such as, for example, the type of pathological processes mediated by JC virus infection, the patient's history and age, the stage of pathological processes mediated by JC virus infection, and the administration of other anti-pathological processes mediated by JC virus infection.

As used herein, a “pharmaceutical composition” includes a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a vector has been introduced from which a dsRNA molecule may be expressed.

### II. DOUBLE-STRANDED RIBONUCLEIC ACID (dsRNA)

In one embodiment, the invention provides double-stranded ribonucleic acid (dsRNA) molecules for inhibiting the expression of a gene from the JC Virus in a cell or mammal, where the dsRNA includes an antisense strand having a region of complementarity that is complementary to at least a part of an mRNA formed in the expression of a gene from the JC Virus, and where the region of complementarity is less than 30 nucleotides in length, generally 19-24 nucleotides in length, and where the dsRNA, upon contact with a cell expressing the gene from the JC virus, inhibits expression of the JC virus gene by at least 40%.

The dsRNA includes two RNA strands that are sufficiently complementary to hybridize to form a duplex structure. One strand of the dsRNA (the antisense strand) includes a region of complementarity that is substantially complementary, and generally fully complementary, to a target sequence derived from the sequence of an mRNA formed during the expression of a gene from the JC Virus. The other strand of the dsRNA (the sense strand) includes a region that is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. In some embodiments, the dsRNA is between 10 and 15 nucleotides in length, and in other embodiments, the dsRNA is between 25 and 30 nucleotides in length. The dsRNA featured in the invention may further include one or more single-stranded nucleotide overhangs.

The dsRNA can be synthesized by standard methods known in the art as further discussed below, e.g., by use of an automated DNA synthesizer, such as are commercially available from, for example, Biosearch, Applied Biosystems, Inc. In one embodiment, a gene from the JC Virus is the human JC virus genome. In specific embodiments, the sense strand of the dsRNA includes a sequence selected from the sense sequences of Tables 8, 10, and 13-16, and the antisense strand of the dsRNA includes a sequence selected from the antisense sequences of Tables 8, 10, and 13-16. Alternative antisense sequences that target elsewhere in the target sequence provided in Tables 8, 10, and 13-16 can readily be determined using the target sequence and the flanking JC virus sequence.

In further embodiments, the dsRNA includes at least one nucleotide sequence selected from the sequences provided in Tables 8, 10, and 13-16. In other embodiments, the dsRNA includes at least two sequences selected from this group, where one of the at least two sequences is complementary to another of the at least two sequences, and one of the at least two sequences is substantially complementary to a sequence of an mRNA generated in the expression of a gene from the JC Virus. Generally, the dsRNA includes two oligonucleotides, where one oligonucleotide is described as a sense strand selected from the sense strands of Tables 8, 10, and 13-16, a second oligonucleotide is described as an antisense strand selected from the antisense strands of Tables 8, 10, and 13-16.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well. In the embodiments described above, by virtue of the nature of the oligonucleotide sequences provided in Tables 8, 10, and 13-16, the dsRNAs featured in the invention can include at least one strand of a length of minimally 21 nt. It can be reasonably expected that shorter dsRNAs having one of the sequences of Tables 8, 10, and 13-16 minus only a few nucleotides on one or both ends may be similarly effective as compared to the dsRNAs described above. Hence, dsRNAs having a partial sequence of at least 15, 16, 17, 18, 19, 20, or more contiguous nucleotides from one of the sequences of Tables 8, 10, and 13-16, and differing in their ability to inhibit the expression of a gene from the JC Virus in a FACS assay as described herein by not more than 5, 10, 15, 20, 25, or 30% inhibition from a dsRNA having the full sequence, are contemplated by the invention. Further, dsRNAs that cleave within the target sequence of a dsRNA provided in Tables 8, 10, or 13-16 can readily be made using the JC virus sequence and the target sequence provided.

In addition, the RNAi agents provided in Tables 8, 10, and 13-16 identify a site in the JC virus mRNA that is susceptible to RNAi based cleavage. As such the present invention further includes RNAi agents that target within the sequence targeted by one of the agents of the present invention. As used herein a second RNAi agent is said to target within the sequence of a first RNAi agent if the second RNAi agent cleaves the message anywhere within the mRNA that is complementary to the antisense strand of the first RNAi agent. Such a second agent will generally consist of at least 15 contiguous nucleotides from one of the sequences provided in Tables 8, 10, and 13-16 coupled to additional nucleotide sequences taken from the region contiguous to the selected sequence in a gene from the JC Virus. For example, the last 15 nucleotides of SEQ ID NO:1 combined with the next 6 nucleotides from the target JC virus genome produces a single strand agent of 21 nucleotides that is based on one of the sequences provided in Tables 8, 10, and 13-16.

A dsRNA targeting a gene of the JC virus can contain one or more mismatches to the target sequence. In one embodiment, the dsRNA contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide dsRNA strand which is complementary to a region of a gene from the JC Virus, the dsRNA generally does not contain any mismatch within the central 13 nucleotides. The methods described herein can be used to determine whether a dsRNA containing a mismatch to a target sequence is effective in inhibiting the expression of a gene from the JC Virus. Consideration of the efficacy of dsRNAs with mismatches in inhibiting expression of a gene from the JC Virus is important, especially if the particular region of complementarity in a gene from the JC Virus is known to have polymorphic sequence variation within the population.

In one embodiment, at least one end of the dsRNA has a single-stranded nucleotide overhang of 1 to 4, generally 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Such dsRNAs have improved stability and inhibitory activity, thus allowing administration at low dosages, i.e., less than 5 mg/kg body weight of the recipient per day. In one embodiment, the antisense strand of the dsRNA has 1-10 nucleotides overhangs each at the 3′ end and the 5′ end over the sense strand. In one embodiment, the sense strand of the dsRNA has 1-10 nucleotides overhangs each at the 3′ end and the 5′ end over the antisense strand. In another embodiment, one or more of the nucleotides in the overhang is replaced with a nucleoside thiophosphate.

In yet another embodiment, the dsRNA is chemically modified to enhance stability. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Specific examples of dsRNA compounds useful in this invention include dsRNAs containing modified backbones or no natural internucleoside linkages. As defined in this specification, dsRNAs having modified backbones include those that retain a phosphorus atom in the backbone and those that do not have a phosphorus atom in the backbone. For the purposes of this specification, and as sometimes referenced in the art, modified dsRNAs that do not have a phosphorus atom in their internucleoside backbone can also be considered to be oligonucleosides.

Typical modified dsRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference

Typical modified dsRNA backbones that do not include a phosphorus atom therein have backbones that are formed by short chain alkyl or cycloalkyl internucleoside linkages, mixed heteroatoms and alkyl or cycloalkyl internucleoside linkages, or ore or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other typical dsRNA mimetics, both the sugar and the internucleoside linkage, i.e., the backbone, of the nucleotide units are replaced with novel groups. The base units are maintained for hybridization with an appropriate nucleic acid target compound. One such oligomeric compound, an dsRNA mimetic that has been shown to have excellent hybridization properties, is referred to as a peptide nucleic acid (PNA). In PNA compounds, the sugar backbone of an dsRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most embodiments featured in the invention include dsRNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. Also included are dsRNAs having morpholino backbone structures of the above-referenced U.S. Pat. No. 5,034,506.

Modified dsRNAs may also contain one or more substituted sugar moieties. Typical dsRNAs include one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Typical embodiments include O[(CH₂)ₙO]ₘCH₃, O(CH₂)ₙOCH₃, O(CH₂)ₙNH₂, O(CH₂)CH₃, O(CH₂)ₙONH₂, and O(CH₂)ₙON[(CH₂)ₙCH₃)]₂, where n and m are from 1 to about 10. Other typical dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. One typical modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxy-alkoxy group. Another typical modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₃)₂, also described in examples hereinbelow.

Other modifications include 2′-methoxy (2′-OCH₃), 2′-aminopropoxy (2′-OCH₂CH₂CH₂NH₂) and 2′-fluoro (2′-F). Similar modifications may also be made at other positions on the dsRNA, particularly the 3′ position of the sugar on the 3′ terminal nucleotide or in 2′-5′ linked dsRNAs and the 5′ position of 5′ terminal nucleotide. DsRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

dsRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, DsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., DsRNA Research and Applications, CRC Press, Boca Raton, 1993, pp. 276-278) and represent typical base substitutions, particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference, and U.S. Pat. No. 5,750,692, also herein incorporated by reference.

Another modification of the dsRNAs featured in the invention involve chemically linking to the dsRNA one or more moieties or conjugates which enhance the activity, cellular distribution or cellular uptake of the dsRNA. Such moieties include but are not limited to lipid moieties such as a cholesterol moiety (Letsinger et al., Proc. Natl. Acid. Sci. USA, 199, 86, 6553-6556), cholic acid (Manoharan et al., Biorg. Med. Chem. Let., 1994 4 1053-1060), a thioether, e.g., beryl-5-tritylthiol (Manoharan et al., Ann. N.Y. Acad. Sci., 1992, 660, 306-309; Manoharan et al., Biorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyloxycholesterol moiety (Crooke et al., J. Pharmacol. Exp. Ther., 1996, 277, 923-937).

Representative U.S. patents that teach the preparation of such dsRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an dsRNA. The present invention also includes dsRNA compounds which are chimeric compounds. “Chimeric” dsRNA compounds or “chimeras,” in the context of this invention, are dsRNA compounds, particularly dsRNAs, which contain two or more chemically distinct regions, each made up of at least one monomer unit, i.e., a nucleotide in the case of an dsRNA compound. These dsRNAs typically contain at least one region wherein the dsRNA is modified so as to confer upon the dsRNA increased resistance to nuclease degradation, increased cellular uptake, and/or increased binding affinity for the target nucleic acid. An additional region of the dsRNA may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of dsRNA inhibition of gene expression. Consequently, comparable results can often be obtained with shorter dsRNAs when chimeric dsRNAs are used, compared to phosphorothioate deoxydsRNAs hybridizing to the same target region. Cleavage of the RNA target can be routinely detected by gel electrophoresis and, if necessary, associated nucleic acid hybridization techniques known in the art.

In certain instances, the dsRNA may be modified by a non-ligand group. A number of non-ligand molecules have been conjugated to dsRNAs in order to enhance the activity, cellular distribution or cellular uptake of the dsRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such dsRNA conjugates have been listed above. Typical conjugation protocols involve the synthesis of dsRNAs bearing an aminolinker at one or more positions of the sequence. The amino group is then reacted with the molecule being conjugated using appropriate coupling or activating reagents. The conjugation reaction may be performed either with the dsRNA still bound to the solid support or following cleavage of the dsRNA in solution phase. Purification of the dsRNA conjugate by HPLC typically affords the pure conjugate.

Vector Encoded RNAi Agents

In another aspect, JC virus specific dsRNA molecules that modulate JC virus genome expression activity are expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In one embodiment, a dsRNA is expressed as an inverted repeat joined by a linker polynucleotide sequence such that the dsRNA has a stem and loop structure.

The recombinant dsRNA expression vectors are generally DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Eglitis, et al., Science (1985) 230:1395-1398; Danos and Mulligan, Proc. NatI. Acad. Sci. USA (1998) 85:6460-6464; Wilson et al., 1988, Proc. NatI. Acad. Sci. USA 85:3014-3018; Armentano et al., 1990, Proc. NatI. Acad. Sci. USA 87:61416145; Huber et al., 1991, Proc. NatI. Acad. Sci. USA 88:8039-8043; Ferry et al., 1991, Proc. NatI. Acad. Sci. USA 88:8377-8381; Chowdhury et al., 1991, Science 254:1802-1805; van Beusechem. et al., 1992, Proc. Nad. Acad. Sci. USA 89:7640-19; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

Any viral vector capable of accepting the coding sequences for the dsRNA molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference.

Typical viral vectors are those derived from AV and AAV. In one embodiment, a dsRNA featured in the invention is expressed as two separate, complementary single-stranded RNA molecules from a recombinant AAV vector comprising, for example, either the U6 or H1 RNA promoters, or the cytomegalovirus (CMV) promoter.

A suitable AV vector for expressing a dsRNA, a method for constructing the recombinant AV vector, and a method for delivering the vector into target cells, are described in Xia H et al. (2002), Nat. Biotech. 20: 1006-1010.

Suitable AAV vectors for expressing the dsRNA, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

The promoter driving dsRNA expression in either a DNA plasmid or viral vector may be a eukaryotic RNA polymerase I (e.g. ribosomal RNA promoter), RNA polymerase II (e.g. CMV early promoter or actin promoter or U1 snRNA promoter) or generally RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Generally, recombinant vectors capable of expressing dsRNA molecules are delivered as described below and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single JC virus genome or multiple JC virus genomes over a period of a week or more are also contemplated by the invention. Successful introduction of vectors into host cells can be monitored using various known methods. For example, transient transfection can be monitored using a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The JC virus specific dsRNA molecules can also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### III. PHARMACEUTICAL COMPOSITIONS COMPRISING dsRNA

In one embodiment, the invention provides pharmaceutical compositions suitable for localized delivery to oligodendrocytes, such as oligodendrocytes of the corpus callosum. Such compositions include a dsRNA as described herein and a pharmaceutically acceptable carrier. The pharmaceutical composition having the dsRNA is useful for treating a disease or disorder associated with the expression or activity of a gene from the JC Virus and/or viral infection, such as PML. Such pharmaceutical compositions are formulated based on the mode of delivery. One example is a composition formulated for infusion directly into the corpus callosum.

Pharmaceutical compositions featured herein can be used for infusion into other areas of white matter, or into the striatum of the brain. Intrastriatal infusion is particularly relevant for treatment of neurological disorders such as Huntington's disease.

The pharmaceutical compositions featured herein are administered in dosages sufficient to inhibit expression of a gene from the JC Virus. In general, a suitable dose of dsRNA will be in the range of 0.01 to 200.0 milligrams per kilogram body weight of the recipient per day, generally in the range of 0.02 to 50 mg per kilogram body weight per day. For example, the dsRNA can be administered at 0.01 mg/kg, 0.1 mg/kg, 0.05 mg/kg, 0.5 mg/kg, 1 mg/kg, 2 mg/kg, 3 mg/kg, 10 mg/kg, 20 mg/kg, 30 mg/kg, 40 mg/kg, or 50 mg/kg per single dose. The pharmaceutical composition may be administered once daily, or the dsRNA may be administered as two, three, or more sub-doses at appropriate intervals throughout the day or even using continuous infusion or delivery through a controlled release formulation. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. In this embodiment, the dosage unit contains a corresponding multiple of the daily dose. In some embodiments, the dsRNA is administered daily, weekly, biweekly, or monthly.

In one embodiment, the dsRNA composition is infused directly into the corpus callosum for 1, 2, 3, 5, or 7 days or more. Methods of intracranial infusion are known in the art and can include, for example, use of an osmotic pump.

The present invention includes pharmaceutical compositions that can be delivered by injection directly into the brain. The injection can be by stereotactic injection into a particular region of the brain (e.g., white matter such as the corona radiata, or the substantia nigra, cortex, hippocampus, striatum, or globus pallidus), or the dsRNA can be delivered into multiple regions of the central nervous system (e.g., into multiple regions of the brain, and/or into the spinal cord). The dsRNA can also be delivered into diffuse regions of the brain (e.g., diffuse delivery to the cortex of the brain).

In one embodiment, a dsRNA targeting a gene expressed in the brain can be delivered by way of a cannula or other delivery device having one end implanted in the brain, e.g., white matter such as the corona radiata, or the substantia nigra, cortex, hippocampus, striatum, corpus callosum or globus pallidus of the brain. The cannula can be connected to a reservoir of the dsRNA composition. The flow or delivery can be mediated by a pump. In one embodiment, a pump and reservoir are implanted in an area distant from the tissue, e.g., in the abdomen, and delivery is effected by a conduit leading from the pump or reservoir to the site of release. Infusion of the dsRNA composition into the brain can be over several hours or for several days, e.g., for 1, 2, 3, 5, or 7 days or more. Devices for delivery to the brain are described, for example, in U.S. Pat. Nos. 6,093,180, and 5,814,014. In another embodiment, the pump is externalized (not implanted). Infusion of the dsRNA composition into the brain can be over several hours or for several days up to approximately 7 days, e.g., for 1, 2, 3, 5, or 7 days.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or series of treatments. Estimates of effective dosages and in vivo half-lives for individual dsRNAs can be made using conventional methodologies or determined on the basis of in vivo testing using an appropriate animal model, as described elsewhere herein.

Advances in mouse genetics have generated a number of mouse models for the study of various human diseases, such as pathological processes mediated by JC virus expression. Such models are used for in vivo testing of dsRNA, as well as for determining a therapeutically effective dose.

The present invention also includes methods of administering pharmaceutical compositions and formulations which include dsRNA compounds, such as those that target an RNA expressed by a pathogen, such as the JC virus.

Compositions and formulations for oral administration include powders or granules, microparticulates, nanoparticulates, suspensions or solutions in water or non-aqueous media. Thickeners, diluents, emulsifiers, dispersing aids or binders may be desirable.

Compositions and formulations for intracranial, parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions for use with methods featured in the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations for use with the methods of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Further advantages of liposomes include the following. Liposomes obtained from natural phospholipids are biocompatible and biodegradable; liposomes can incorporate a wide range of water and lipid soluble drugs; and liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes including one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes including (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes including lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Ilium et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes including phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes containing nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an dsRNA RNA. U.S. Pat. No. 5,665,710 to Rahman et al. describes certain methods of encapsulating oligodeoxynucleotides in liposomes. WO 97/04787 to Love et al. discloses liposomes comprising dsRNA dsRNAs targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Agents that enhance uptake of dsRNAs at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions suitable for intracranial administration also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′ isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions suitable for intracranial administration may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments provide the use of pharmaceutical compositions containing (a) one or more antisense compounds and (b) one or more other antiviral agents that function by a non-antisense mechanism. Examples of such antiviral agents include but are not limited to members of classes of agents including reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, ribivirin, vidarabine, indinavir, nelfinavir, amprenavir, zidovudine (AZT), stavudine (d4T), larnivudine (3TC), didanosine (DDI), zalcitabine (ddC), abacavir, acyclovir, penciclovir, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside. When used with the dsRNAs featured in the invention, such antiviral agents may be used individually, sequentially, or in combination with one or more other such antiviral agents. Anti-inflammatory drugs, including but not limited to nonsteroidal anti-inflammatory drugs and corticosteroids may also be combined in compositions. See, generally, The Merck Manual of Diagnosis and Therapy, 15th Ed., Berkow et al., eds., 1987, Rahway, N.J., pages 2499-2506 and 46-49, respectively). Other non-antisense antiviral agents are also within the scope of this invention. Two or more combined compounds may be used together or sequentially.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are typical.

The data obtained from cell culture assays and animal studies can be used in formulating a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, dsRNAs targeting JC virus can be administered in combination with other known agents effective in treatment of pathological processes mediated by JC virus expression. In any event, the administering physician can adjust the amount and timing of dsRNA administration on the basis of results observed using standard measures of efficacy known in the art or described herein.

Methods for Treating Diseases Caused by Expression of a Gene in the Brain

The invention relates in particular to the administration of a dsRNA or a pharmaceutical composition prepared therefrom for the treatment or prevention of pathological conditions associated with gene expression in the brain, such as from a JC virus infection, e.g., PML, or a brain tumor, such as glioblastoma multiforme. Owing to the inhibitory effect on gene expression, e.g., JC virus expression, a dsRNA according to the invention or a pharmaceutical composition prepared therefrom can enhance the quality of life, particularly in a patient being treated with an anti-VLA4 antibody (e.g., Tysabri™) as part of treatment for MS.

Administration is typically to the central nervous system of the individual, such as into oligodendrocytes, e.g., oligodendrocytes of the corpus callosum. Administration can also be to other areas of white matter in the brain, or into the striatum, such as by intrastriatal infusion.

The invention also relates to the use of a dsRNA or a pharmaceutical composition thereof for treating PML in combination with other pharmaceuticals and/or other therapeutic methods, e.g., with known pharmaceuticals and/or known therapeutic methods, such as, for example, those which are currently employed for treating and/or preventing viral infection.

In some embodiments, dsRNAs targeting CNPase are useful for treating a neurological disorder, e.g., schizophrenia, Down Syndrome, Alzheimer's Disease or Huntington's Disease. In other embodiments, dsRNAs targeting a gene expressed in the corpus callosum, e.g., in oligodendrocytes of the corpus callosum are useful for treatment of, for example, Parkinson's disease, multiple sclerosis, amyotrophic lateral sclerosis, autoimmune encephalomyelitis, stroke, or Huntington's disease.

The dsRNA and an additional therapeutic agent can be administered in the same combination, e.g., intracranially, such as into the corpus callosum, or the additional therapeutic agent can be administered as part of a separate composition, intracranially or by another method described herein.

The invention features a method of administering a dsRNA directly to a cell in the brain of a mammal, e.g., to an oligodendrocyte in the brain of a mammal. The dsRNA can target a gene of the JC virus, for example, in a patient infected with JC virus. Patients can be administered a therapeutic amout of dsRNA, such as 0.2 mg/kg, 0.1 mg/kg, 0.5 mg/kg, 1.0 mg/kg, 1.5 mg/kg, 2.0 mg/kg, or 2.5 mg/kg dsRNA. The dsRNA can be administered by intravenous infusion over a period of time, such as over a 5 minute, 10 minute, 15 minute, 20 minute, or 25 minute period. The administration is repeated, for example, on a regular basis, such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Administration of the dsRNA can reduce JC virus mRNA levels in a sample of the patient, e.g., a blood sample, by at least 20%, 25%, 30%, 40%, 50%, 60%, 70%, 80% or 90% or more.

In some embodiments, the dsRNA can be administered by intracranial infusion over a period of time, such as over a 30 minute, 1 hour, 2 hour, 3 hour or 4 hour period. The administration can be repeated, for example, on a regular basis, such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Intracranial infusion can be continous.

Before administration of a full dose of the dsRNA, patients can be administered a smaller dose, such as a 5% infusion reaction, and monitored for adverse effects, such as an allergic reaction

Many neurodegenerative-associated diseases and disorders are hereditary. Therefore, a patient in need of a dsRNA targeting a gene expressed in the corpus callosum can be identified by taking a family history. A healthcare provider, such as a doctor, nurse, or family member, can take a family history before prescribing or administering a dsRNA. A DNA test may also be performed on the patient to identify a mutation in the target gene, before a dsRNA is administered to the patient.

The invention can also be practiced by including with a specific RNAi agent, in combination with another anti-viral agent, such as any conventional anti-viral agent. The combination of a specific binding agent with such other agents can potentiate the anti-viral protocol. Thus, methods can be employed with such conventional regimens with the benefit of reducing side effects and enhancing efficacy.

Methods for Inhibiting Expression of a Gene from the JC Virus

In yet another aspect, the invention provides a method for inhibiting the expression of a gene from the JC Virus in a mammal. The method includes administering a dsRNA to the mammal such that expression of the target JC virus genome is silenced. Because of their high specificity, the dsRNAs featured in the invention specifically target RNAs (primary or processed) of the target JC virus gene. Compositions and methods for inhibiting the expression of these JC virus genes using dsRNAs can be performed as described elsewhere herein.

In one embodiment, the method includes administering a composition containing a dsRNA, where the dsRNA includes a nucleotide sequence that is complementary to at least a part of an RNA transcript of a gene from the JC Virus, to the mammal to be treated. The compositions are administered into oligodendrocytes, such as into oligodendrocytes within the corpus callosum.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Design of JCV siRNAs

Full-length genome sequences to JC virus available on Apr. 10, 2006, were obtained, resulting in a target pool of 388 sequences (accession numbers: AB038249.1-AB038255.1; AB048545.1-AB048582.1; AB074575.1-AB074591.1; AB077855.1-AB077879.1; AB081005.1-AB081030.1; AB081600.1-AB081618.1; AB081654.1; AB092578.1-AB092587.1; AB103387.1; AB103402.1-AB103423.1; AB104487.1; AB113118.1-AB113145.1; AB118651.1-AB118659.1; AB126981.1-AB127027.1; AB127342.1; AB127344.1; AB127346.1-AB127349.1; AB127352.1-AB127353.1; AB198940.1-AB198954.1; AB220939.1-AB220943.1; AF004349.1-AF004350.1; AF015526.1-AF015537.1; AF015684.1; AF030085.1; AF281599.1-AF281626.1; AF295731.1-AF295739.1; AF300945.1-AF300967.1; AF363830.1-AF363834.1; AF396422.1-AF396435.1; AY121907.1-AY121915.1; NC_{—}001699.1; U61771.1; U73500.1-U73502.1). NC_{—}001699 was defined as reference sequence.

The siRNA selection process was run as follows: ClustalW multiple alignment was used to generate a global alignment of all sequences from the target pool. An IUPAC consensus sequence was then generated.

All conserved 19 mer target sequences from the IUPAC consensus represented by stretches containing only A, T, C or G bases, which are therefore present in all sequences of the target pool were selected. In order to only select siRNAs that target transcribed sequence parts of the JC virus, candidate target sequences were selected out of the pool of conserved 19 mer target sequences. For this, candidate target sequences covering regions between nucleotide 163-2594 and between 2527-5115 relative to reference sequence were extracted for late and early genes, respectively. Further, as sequences for early genes are in reverse complement orientation compared with genomic sequences, candidate target sequences of these genes were transferred to reverse complement sequences and replaced the former pool of candidate target sequences.

In order to rank candidate target sequences and their respective siRNAs and select appropriate ones, their predicted potential for interacting with irrelevant targets (off-target potential) was taken as a ranking parameter. siRNAs with low off-target potential were defined as preferable and assumed to be more specific in vivo.

For predicting siRNA-specific off-target potential, the following assumptions were made:
1) positions 2 to 9 (counting 5′ to 3′) of a strand (seed region) may contribute more to off-target potential than rest of sequence (non-seed and cleavage site region)
2) positions 10 and 11 (counting 5′ to 3′) of a strand (cleavage site region) may contribute more to off-target potential than non-seed region
3) an off-target score can be calculated for each hit, based on identity to siRNA sequence and position of mismatches
4) assuming potential abortion of sense strand activity by internal modifications introduced, only off-target potential of antisense strand will be relevant

To identify potential off-target genes, 19 mer input sequences were subjected to a homology search against publically available human mRNA sequences.

To this purpose, fastA (version 3.4) searches were performed with all 19 mer candidate target sequences against a human RefSeq database (downloaded available version from ftp://ftp.ncbi.nih gov/refseq/on Nov. 7, 2006). FastA searches were executed with parameters-values-pairs—f 50-g 50 in order to take into account the homology over the full length of the 19 mer without any gaps. In order to ensure the listing of all relevant off-target hits in the fastA output file the parameter—E 30000 was used in addition. A scoring matrix was applied for the run that assessed every nucleotide match with a score of 13 and every mismatch with a score of −7. The search resulted in a list of potential off-targets for each candidate siRNA.

To sort the resulting list of potential off-targets for each siRNA, fastA output files were analyzed to identify the best off-target and its off-target score. The following off-target properties for each 19 mer input sequence were extracted for each off-target to calculate the off-target score:
Number of mismatches in non-seed region
Number of mismatches in seed region
Number of mismatches in cleavage site region

The off-target score was calculated for considering assumption 1 to 3 as follows:

$$Off \text{-} target score = number of seed mismatches * 10 + number of cleavage site mismatches * 1.2 + number of non \text{-} seed mismatches * 1$$

The most relevant off-target gene for input each 19 mer input sequences was defined as the gene with the lowest off-target score. Accordingly, the lowest off-target score was defined as the relevant off-target score for the corresponding siRNA.

In order to generate a ranking for siRNAs, calculated relevant off-target scores were transferred into a result table. All siRNAs were sorted according to the off-target score (descending).

An off-target score of 2.2 was defined as cut-off for siRNA selection (specificity criterion). In addition, all sequences with only one mismatch in the seed region were eliminated from the screening set. The selection procedure resulted in a set of 93 JCV specific siRNAs (Table 8).

An expanded screening was generated by re-calculating the predicted specificity based on the newly available human RefSeq database (Human mRNA sequences in RefSeq release version 21 (downloaded Jan. 12, 2007)) and selecting only 208 siRNAs that did not contain more than 3 G's in a row and had an off-target score of at least 2 for the antisense strand (Table 10).

Synthesis of JCV siRNAs

All siRNAs were synthesized in 0.2 μmole synthesis scale on an ABI3900 DNA synthesizer according to standard procedures.

For the initial screening set (93 different siRNA sequences), 4 different strategies of chemical modification were used:
a) exo/endo light (EEL): -sense strand: 2′-O-methyl @ all pyrimidines, PTO between nucleotides 20 and 21 (counting from 5′-end), dTdT at 3′-end (nucleotides 20 and 21)
   antisense strand: 2′-O-methyl at pyrimidines in 5′-UA-3′ and 5′-CA-3′ motives, PTO between nucleotides 20 and 21 (counting from 5′-end), dTdT at 3′-end (nucleotides 20 and 21)
b) EEL plus 2′-O-methyl in position 2 of antisense strand (only if no 5′-UA-3′ and 5′-CA-3′ at 5′-end, otherwise already covered by EEL)
c) EEL plus 2′-O-methyl in position 2 of sense strand (only if no pyrimidine in position 2, otherwise already covered by EEL)
d) EEL plus 2′-O-methyl in position 2 of sense and antisense strand (only if not already covered by a, b, and c) (Table 8)

For the expanded screening set (208 different siRNA sequences), siRNAs were composed of unmodified RNA oligonucleotides with dT/dT overhangs (dTdT at 3′-end (nucleotides 20 and 21) of antisense and sense strands) (Table 10).

Synthesis of Conjugated siRNAs

siRNAs conjugated to Vitamine E were prepared according to schemes 1 and 2. It is understood that other conjugates can be linked to the oligonucleotides via a similar method known to one of ordinary skill in the art, such methods can be found in U.S. publication nos. 2005/0107325, 2005/0164235, 2005/0256069 and 2008/0108801, which are hereby incorporated by their entirety.

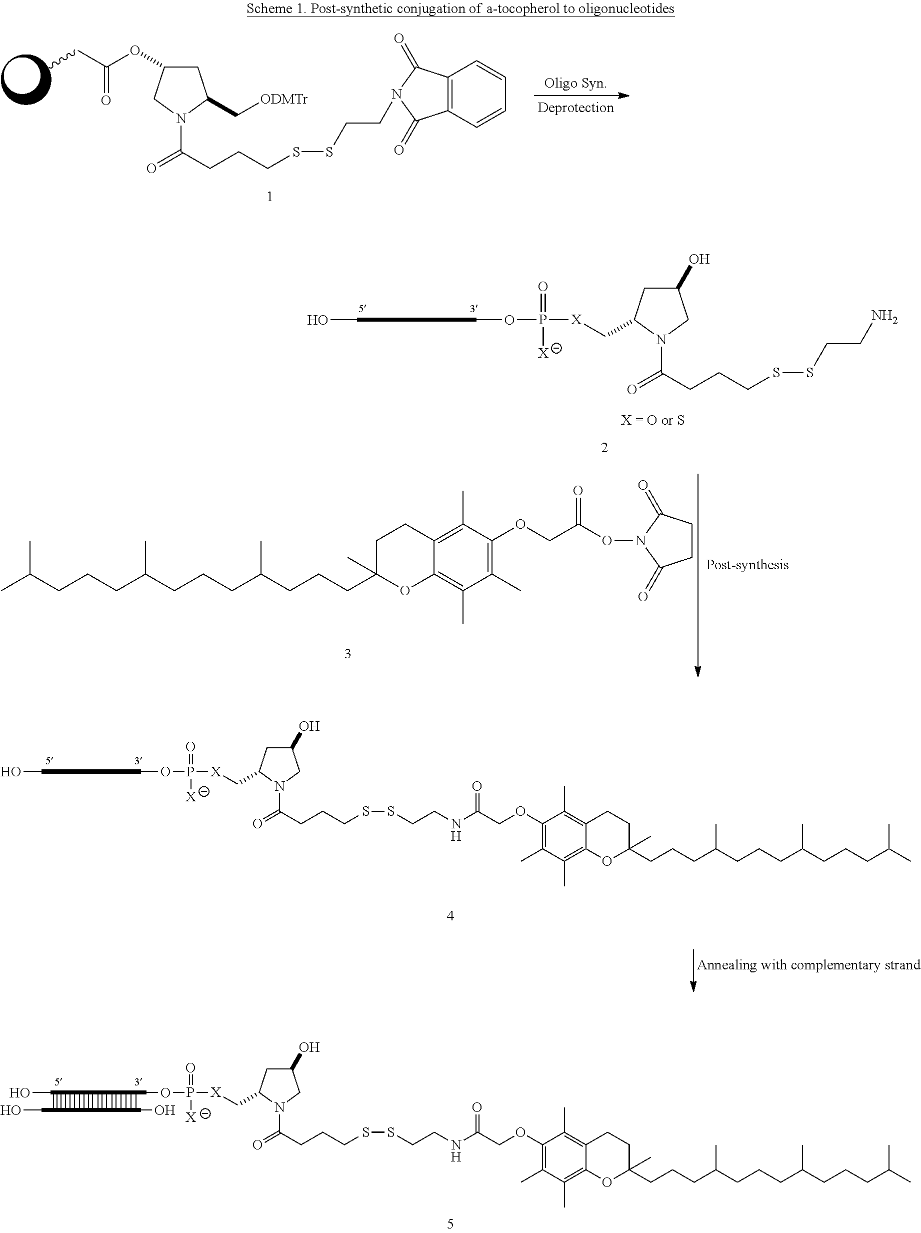

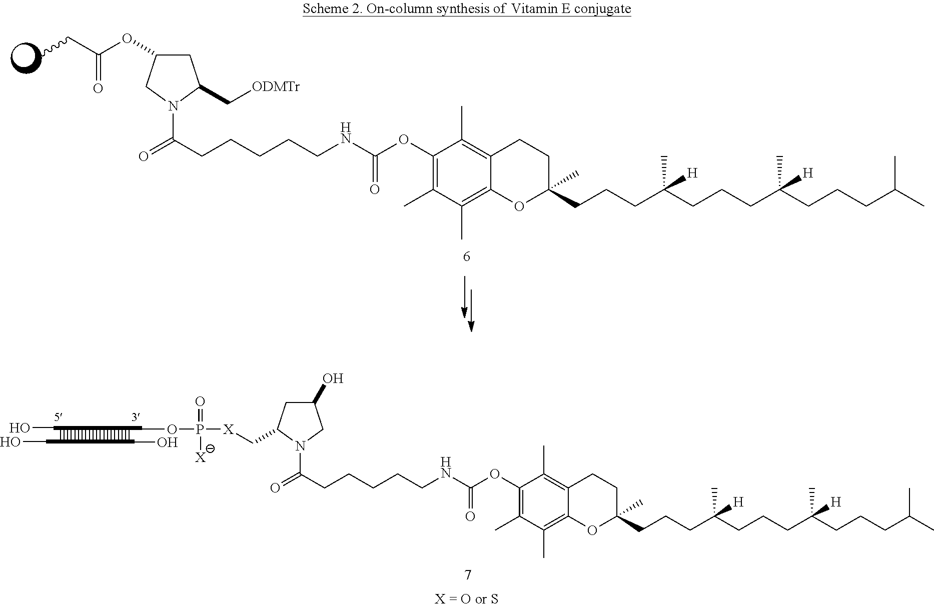

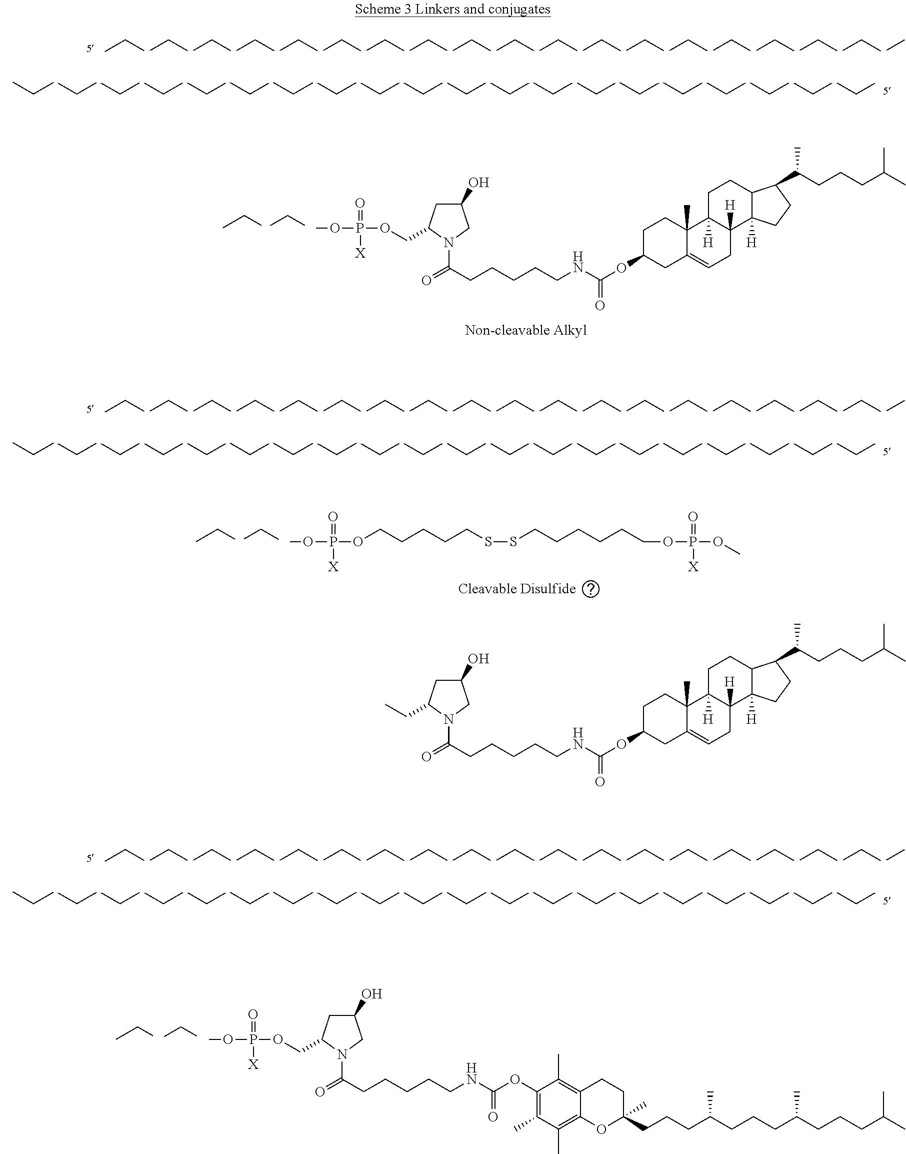

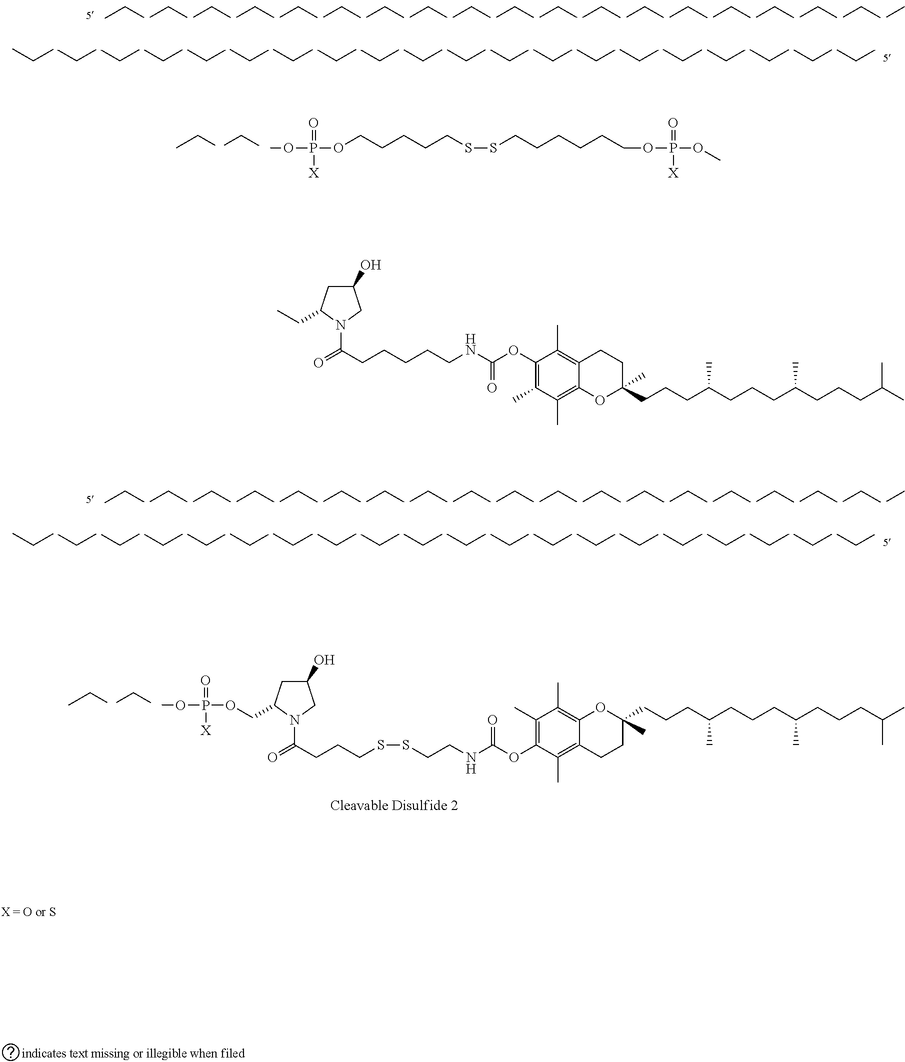

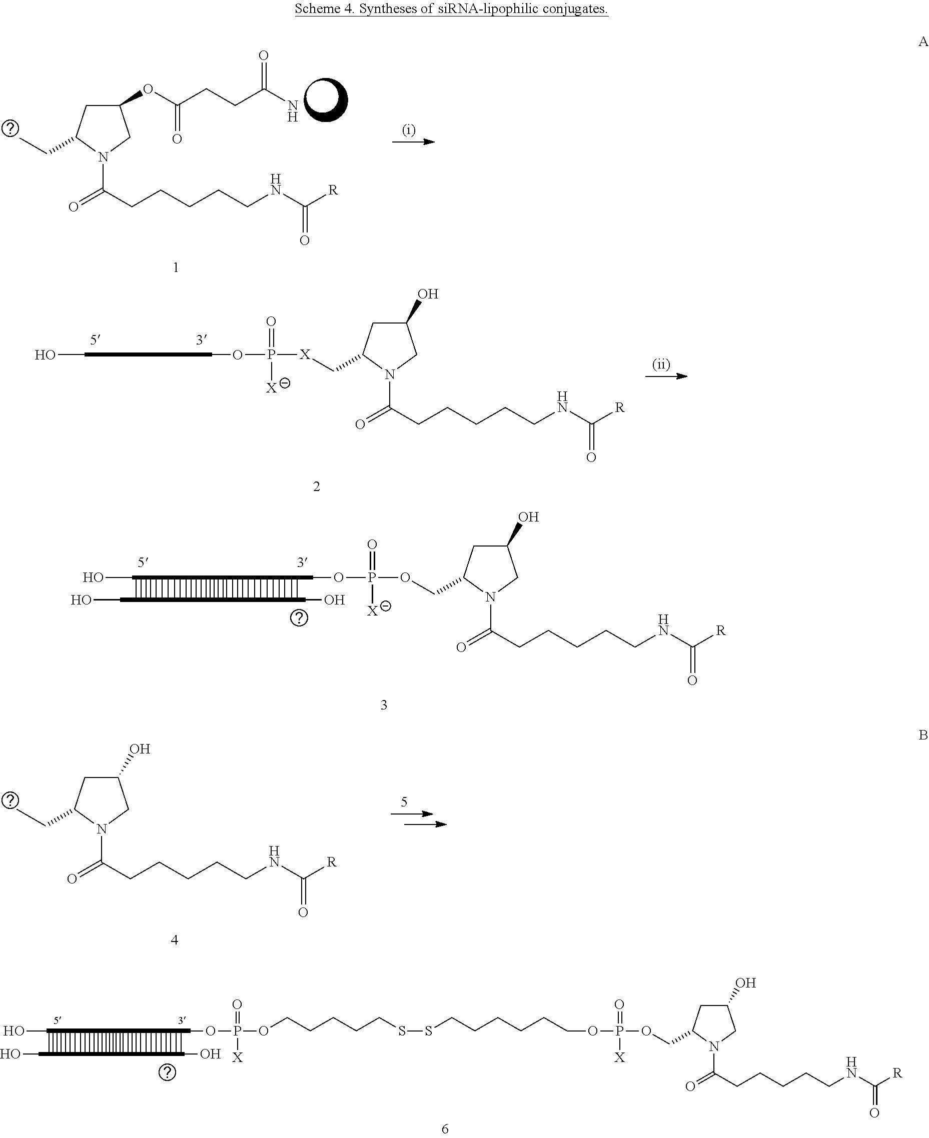

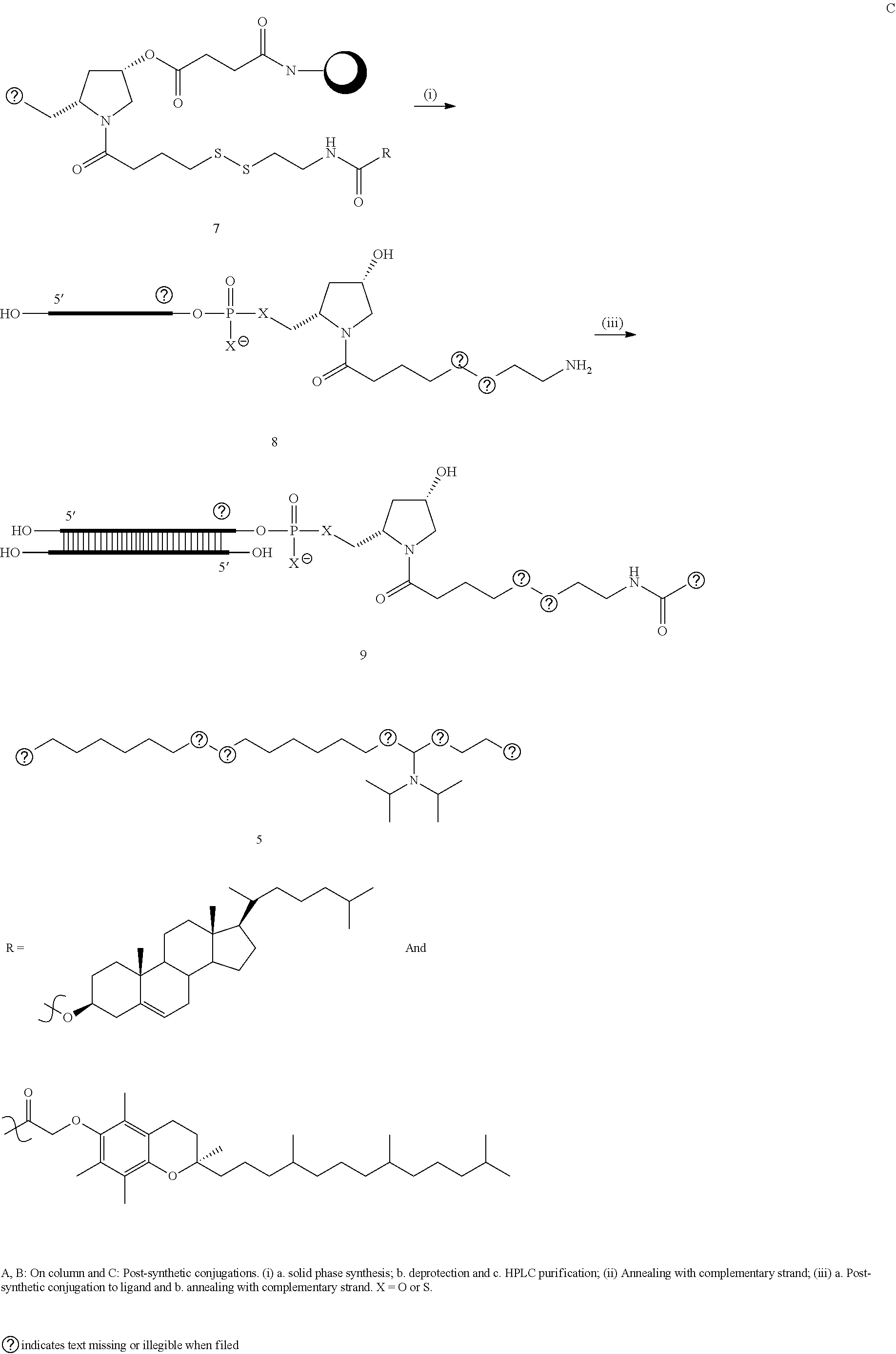

TABLE 1
Sequence of conjugated CNPase dsRNAs

| | SEQ | | | |
|---|---|---|---|---|
| | ID | | SEQ | |
| Duplex | NO: | Sense (5′ to 3′) | ID NO: | Antisense (5′ to 3′) |
| AD-3178 | 2045 | GGccuuGAccucuuAGAGAdTdTL10 | 2046 | UCUCuAAGAGGUcAAGGCCTsT |
| AD-3222 | 2047 | GGccuuGAccucuuAGAGAdTdTQ51L10 | 2048 | UCUCuAAGAGGUcAAGGCCTsT |
| AD-3181 | 2049 | GGccuuGAccucuuAGAGAdTdTL13 | 2050 | UCUCuAAGAGGUcAAGGCCTsT |
| AD-3569 | 2051 | GGccuuGAccucuuAGAGAdTdTQ51L13 | 2052 | UCUCuAAGAGGUcAAGGCCTsT |
| AD-18233 | 2053 | GGccuuGAccucuuAGAGAuUUUL13 | 2054 | AAAAUCUCuAAGAGGUcAAGGCCUG |
| AD-18528 | 2055 | GGccuuGAccucuuAGAGAdTdTL99 | 2056 | UCUCuAAGAGGUcAAGGCCTsT |

TABLE 2
Nucleotide designations for dsRNAs

| | |
|---|---|
| A | adenosine-3′-phosphate |
| C | cytidine-3′-phosphate |
| G | guanosine-3′-phosphate |
| T | 5-methyluridine-3′-phosphate |
| U | uridine-3′-phosphate |
| c | 2′-O-methylcytidine-3′-phosphate |
| dT | 2′-deoxythymidine-3′-phosphate |
| u | 2′-O-methyluridine-3′-phosphate |
| Ts | 5-methyluridine-3′-phosphorothioate |
| Y14 | 5-(psoralencarboxamidoethyl-3-acrylimido)thymidine-3′-phosphate |
| Q51 | 6-hydroxyhexyldithiohexylphosphate (Thiol-Modifier |
| | C6 S-S Glen Res. 10-1936) |
| L10 | N-(cholesterylcarboxamidocaproyl)-4-hydroxyprolinol |
| | (Hyp-C6-Chol) |
| L13 | N-(tocopherylcarboxamidocaproyl)-4-hydroxyprolinol |
| | (Hyp-C6-Vit. E) |
| L99 | aminoethyldithiobutyryl)-4-hydroxyprolinol [Hyp-C4-S-S-NH2] |
| L79 | N-(alpha-tocopherylcarboxamidoethyl-dithio-butyryl)-4- |
| | hydroxyprolinol (Hyp-S-S-VitE) |

Screening of JCV siRNAs

Construction of Reporter-Systems Encoding JCV Transcripts

The sequence of the early JCV transcript (E) was synthesized at GENEART (Regensburg, Germany) and cloned into GENEART standard vectors. The sequence of the late JCV transcript was subdivided in a first approach into two fragments: L1, including the transcript sequence of the VP1 protein, and LA23, including the sequences of VP2, VP3 and the Agnoprotein. Due to cloning problems with fragment LA23, this sequence was subdivided in a second approach into two fragments (LA23 1-700 and LA23 701-1438). All sequences were synthesized at GENEART and cloned into GENEART standard vectors. All fragments (E, L1, LA23 1-700 and LA23 701-1438) were subcloned into psiCheck-2 (Promega, Mannheim, Germany) via XhoI and NotI (both NEB, Frankfurt, Germany), resulting in constructs with the JCV sequences between the stop-codon and the polyA-signal of Renilla luciferase.

L1
(SEQ ID NO: 931)

CTCGAGACTTTTAGGGTTGTACGGGACTGTAACACCTGCTCTTGAAGCATATGAAGATGGCCCC
AACAAAAAGAAAAGGAGAAAGGAAGGACCCCGTGCAAGTTCCAAAACTTCTTATAAGAGGAGGA
GTAGAAGTTCTAGAAGTTAAAACTGGGGTTGACTCAATTACAGAGGTAGAATGCTTTTTAACTC
CAGAAATGGGTGACCCAGATGAGCATCTTAGGGGTTTTAGTAAGTCAATTTCTATATCAGATAC
ATTTGAAAGTGACTCCCCAAATAAGGACATGCTTCCTTGTTACAGTGTGGCCAGAATTCCACTA
CCCAATCTAAATGAGGATCTAACCTGTGGAAATATACTAATGTGGGAGGCTGTGACCTTAAAAA
CTGAGGTTCTAGGGGTGACAACTTTGATGAATGTGCACTCTAATGGTCAAGCAACTCATGACAA
TGGTGCAGGAAAGCCAGTGCAGGGCACCAGCTTTCATTTTTTTTCTGTTGGCGGGGAGGCTTTA
GAATTACAGGGGGTGGTTTTTAATTACAGAACAAAGTACCCAGATGGAACAATTTTTCCAAAGA
ATGCAACAGTGCAATCTCAAGTAATGAACACAGAGCACAAGGCGTACCTAGATAAGAACAAAGC
ATATCCTGTTGAATGTTGGGTTCCTGATCCCACCAGAAATGAAAACACAAGATATTTTGGGACA
CTAACAGGAGGAGAAAATGTTCCTCCAGTTCTTCATATAACAAACACTGCCACAACAGTGCTGC
TTGATGAATTTGGTGTTGGGCCACTTTGCAAAGGTGACAACTTGTATTTGTCAGCTGTTGATGT
TTGTGGAATGTTTACTAACAGATCTGGTTCCCAGCAGTGGAGAGGACTGTCCAGATATTTTAAG
GTTCAGCTCAGAAAAAGGAGGGTTAAAAACCCCTACCCAATTTCTTTCCTTCTTACTGATTTGA
TTAACAGAAGGACCCCTAGAGTTGATGGGCAACCTATGTATGGTATGGATGCTCAGGTAGAGGA
GGTTAGAGTTTTTGAGGGGACAGAGGAACTTCCAGGGGACCCAGACATGATGAGATATGTTGAC
AGATATGGACAGTTGCAAACAAAGATGCTGTAATCAAAATCCTTTATTGTAATATGCAGTACAT
TTTAATAAAGTATAACCAGCTTTACTTTACAGTTGCAGTCATGCGGCCGC
E

(SEQ ID NO: 932)
CTCGAGCCGCCTCCAAGCTTACTCAGAAGTAGTAAGGGCGTGGAGGCTTTTTAGGAGGC
CAGGGAAATTCCCTTGTTTTTCCCTTTTTTGCAGTAATTTTTTGCTGCAAAAAGCTAAAATGGA
CAAAGTGCTGAATAGGGAGGAATCCATGGAGCTTATGGATTTATTAGGCCTTGATAGGTCTGCA
TGGGGGAACATTCCTGTCATGAGAAAAGCTTATCTGAAAAAATGCAAAGAACTCCACCCTGATA
AAGGTGGGGACGAAGACAAGATGAAGAGAATGAATTTTTTATATAAAAAAATGGAACAAGGTGT
AAAAGTTGCTCATCAGCCTGATTTTGGTACATGGAATAGTTCAGAGGTTGGTTGTGATTTTCCT
CCTAATTCTGATACCCTTTATTGCAAGGAATGGCCTAACTGTGCCACTAATCCTTCAGTGCATT
GCCCCTGTTTAATGTGCATGCTAAAATTAAGGCATAGAAACAGAAAATTTTTAAGAAGCAGCCC
ACTTGTGTGGATAGATTGCTATTGCTTTGATTGCTTCAGACAATGGTTTGGGTGTGACTTAACC
CAAGAAGCTCTTCATTGCTGGGAGAAAGTTCTTGGAGACACCCCCTACAGGGATCTAAAGCTTT
AAGTGCCAACCTATGGAACAGATGAATGGGAATCCTGGTGGAATACATTTAATGAGAAGTGGGA
TGAAGACCTGTTTTGCCATGAAGAAATGTTTGCCAGTGATGATGAAAACACAGGATCCCAACAC
TCTACCCCACCTAAAAAGAAAAAAAAGGTAGAAGACCCTAAAGACTTTCCTGTAGATCTGCATG
CATTCCTCAGTCAAGCTGTGTTTAGTAATAGAACTGTTGCTTCTTTTGCTGTGTATACCACTAA
AGAAAAAGCTCAAATTTTATATAAGAAACTTATGGAAAAATATTCTGTAACTTTTATAAGTAGA
CATGGTTTTGGGGGTCATAATATTTTGTTTTTCTTAACACCACATAGACATAGAGTGTCAGCAA
TTAATAACTACTGTCAAAAACTATGTACCTTTAGTTTTTTAATTTGTAAAGGTGTGAATAAGGA
ATACTTGTTTTATAGTGCCCTGTGTAGACAGCCATATGCAGTAGTGGAAGAAAGTATTCAGGGG
GGCCTTAAGGAGCATGACTTTAACCCAGAAGAACCAGAAGAAACTAAGCAGGTTTCATGGAAAT
TAGTTACACAGTATGCCTTGGAAACCAAGTGTGAGGATGTTTTTTTGCTTATGGGCATGTACTT
AGACTTTCAGGAAAACCCACAGCAATGCAAAAAATGTGAAAAAAAGGATCAGCCAAATCACTTT
AACCATCATGAAAAACACTATTATAATGCCCAAATTTTTGCAGATAGCAAAAATCAAAAAAGCA
TTTGCCAGCAGGCTGTTGATACTGTAGCAGCCAAACAAAGGGTTGACAGCATCCACATGACCAG
AGAAGAAATGTTAGTTGAAAGGTTTAATTTCTTGCTTGATAAAATGGACTTAATTTTTGGGGCA
CATGGCAATGCTGTTTTAGAGCAATATATGGCTGGGGTGGCCTGGATTCATTGCTTGCTGCCTC
AAATGGACACTGTTATTTATGACTTTCTAAAATGCATTGTATTAAACATTCCAAAAAAAAGGTA
CTGGCTATTCAAGGGGCCAATAGACAGTGGCAAAACTACTTTAGCTGCAGCTTTACTTGATCTC
TGTGGGGGAAAGTCATTAAATGTTAATATGCCATTAGAAAGATTAAACTTTGAATTAGGAGTGG
GTATAGATCAGTTTATGGTTGTATTTGAGGATGTAAAAGGCACTGGTGCAGAGTCAAGGGATTT
ACCTTCAGGGCATGGCATAAGCAACCTTGATTGCTTAAGAGATTACTTAGATGGAAGTGTAAAA
GTTAATTTAGAGAGAAAACACCAAAACAAAAGAACACAGGTGTTTCCACCTGGAATTGTAACCA
TGAATGAATATTCAGTGCCTAGAACTTTACAGGCCAGATTTGTAAGGCAGATAGATTTTAGACC
AAAGGCCTACCTGAGAAAATCACTAAGCTGCTCTGAGTATTTGCTAGAAAAAAGGATTTTGCAA
AGTGGTATGACTTTGCTTTTGCTTTTAATCTGGTTTAGGCCAGTTGCTGACTTTGCAGCTGCCA
TTCATGAGAGGATTGTGCAGTGGAAAGAAAGGCTGGATTTAGAAATAAGCATGTATACATTTTC
TACTATGAAAGCTAATGTTGGTATGGGGAGACCCATTCTTGACTTTCCTAGAGAGGAAGATTCT
GAAGCAGAAGACTCTGGACATGGATCAAGCACTGAATCACAATCACAATGCTTTTCCCAGGTCT
CAGAAGCCTCTGGTGCAGACACACAGGAAAACTGCACTTTTCACATCTGTAAAGGCTTTCAATG
TTTCAAAAAACCAAAGACCCCTCCCCCAAAATAACTGCAACTGTGCGGCCGC
LA23 1-700
(SEQ ID NO: 933)
CTCGAGCAGCTAACAGCCAGTAAACAAAGCACAAGGGGAAGTGGAAAGCAGCCAAGGGAACATG
TTTTGCGAGCCAGAGCTGTTTTGGCTTGTCACCAGCTGGCCATGGTTCTTCGCCAGCTGTCACG
TAAGGCTTCTGTGAAAGTTAGTAAAACCTGGAGTGGAACTAAAAAAAGAGCTCAAAGGATTTTA
ATTTTTTTGTTAGAATTTTTGCTGGACTTTTGCACAGGTGAAGACAGTGTAGACGGGAAAAAAA
GACAGAGACACAGTGGTTTGACTGAGCAGACATACAGTGCTTTGCCTGAACCAAAAGCTACATA
GGTAAGTAATGTTTTTTTTTGTGTTTTCAGGTTCATGGGTGCCGCACTTGCACTTTTGGGGGAC
CTAGTTGCTACTGTTTCTGAGGCTGCTGCTGCCACAGGATTTTCAGTAGCTGAAATTGCTGCTG
GAGAGGCTGCTGCTACTATAGAAGTTGAAATTGCATCCCTTGCTACTGTAGAGGGGATTACAAG
TACCTCTGAGGCTATAGCTGCTATAGGCCTTACTCCTGAAACATATGCTGTAATAACTGGAGCT
CCGGGGGCTGTAGCTGGGTTTGCTGCATTGGTTCAAACTGTAACTGGTGGTAGTGCTATTGCTC
AGTTGGGATATAGATTTTTTGCTGACTGGGATCATAAAGTTTCAACAGTTGGGCTTTTTCGCGG
CCGC
LA23 701-1438
(SEQ ID NO: 934)
CTCGAGAGCAGCCAGCTATGGCTTTACAATTATTTAATCCAGAAGACTACTATGATATTTTATT
TCCTGGAGTGAATGCCTTTGTTAACAATATTCACTATTTAGATCCTAGACATTGGGGCCCGTCC
TTGTTCTCCACAATCTCCCAGGCTTTTTGGAATCTTGTTAGAGATGATTTGCCAGCCTTAACCT
CTCAGGAAATTCAGAGAAGAACCCAAAAACTATTTGTTGAAAGTTTAGCAAGGTTTTTGGAAGA
AACTACTTGGGCAATAGTTAATTCACCAGCTAACTTATATAATTATATTTCAGACTATTATTCT
AGATTGTCTCCAGTTAGGCCCTCTATGGTAAGGCAAGTTGCCCAAAGGGAGGGAACCTATATTT
CTTTTGGCCACTCATACACCCAAAGTATAGATGATGCAGACAGCATTCAAGAAGTTACCCAAAG
GCTAGATTTAAAAACCCCAAATGTGCAATCTGGTGAATTTATAGAAAGAAGTATTGCACCAGGA
GGTGCAAATCAAAGATCTGCTCCTCAATGGATGTTGCCTTTACTTTTAGGGTTGTACGGGACTG
TAACACCTGCTCTTGAAGCATATGAAGATGGCCCCAACAAAAAGAAAAGGAGAAAGGAAGGACC
CCGTGCAAGTTCCAAAACTTCTTATAAGAGGAGGAGTAGAAGTTCTAGAAGTTAAAACTGGGGT
TGACTCAATTACAGAGGTAGAATGCTGCGGCCGC

### Example 2

### Screen of JCV siRNAs in Transfected Cells

Cos-7 cells (DSMZ, Braunschweig, Germany, # ACC-60) were seeded at 1.5×10⁴ cells/well on white 96-well plates with clear bottoms (Greiner Bio-One GmbH, Frickenhausen, Germany) in 75 μl of growth medium. Directly after seeding the cells, 50 ng of the corresponding reporter-plasmid per well was transfected with Lipofectamine™ 2000 (Invitrogen GmbH, Karlsruhe, Germany), with the plasmid diluted in Opti-MEM to a final volume of 12.5 μl per well, prepared as a mastermix for the whole plate.

4 h after plasmid transfection, growth medium was removed from cells and replaced by 100 μl/well of fresh medium. siRNA transfections were performed using Lipofectamine™ 2000 (Invitrogen GmbH, Karlsruhe, Germany) as described by the manufacturer. Cells were incubated for 24 h at 37° C. and 5% CO₂ in a humidified incubator (Heraeus GmbH, Hanau, Germany). For the primary screen, all siRNAs were screened at a final concentration of 30 nM. Selected sequences were rescreened at a siRNA concentration of 300 pM. Each siRNA was tested in quadruplicate for each concentration.

Cells were lysed by removing growth medium and application of 150 μl of a 1:1 mixture consisting of medium and substrate from the Dual-Glo Luciferase Assay System (Promega, Mannheim, Germany). The luciferase assay was performed according to the manufacturer's protocol for Dual-Glo Luciferase assay and luminescence was measured in a Victor-Light 1420 Luminescence Counter (Perkin Elmer, Rodgau-Jügesheim, Germany). Values obtained with Renilla luciferase were normalized to the respective values obtained with Firefly luciferase in order to correct for transfection efficacy. Renilla/Firefly luciferase activities obtained after transfection with siRNAs directed against a JCV gene were normalized to Renilla/Firefly luciferase activities obtained after transfection of an unrelated control siRNA set to 100%. Tables 8, 10, and 13-16 provide the results where the siRNAs, the sequences of which are given in Tables 8, 10, and 13-16, were tested at a single dose of 30 nM. The percentage inhibition±standard deviation, compared to the unrelated control siRNA, is indicated in the column ‘Remaining luciferase activity (% of control)’. A number of JCV siRNAs at 30 nM were effective at reducing levels of the targeted mRNA by more than 70% in Cos-7 cells (i.e. remaining luciferase activity was less than 30%).

Selected JCV siRNAs from the single dose screen were further characterized by dose response curves. Transfections of JCV siRNAs for generation of dose response curves were performed with the following siRNA concentrations according to the above protocol:
from 33 nM in 3-fold dilutions down to 0.005 nM (for fragment L1)
from 24 nM in 4-fold dilutions down to 0.001 nM (for fragment E and fragments LA23 1-700 and LA23 701-1438).

IC50 values were determined by parameterized curve fitting using the program XLfit (IDBS, Guildford, Great Britain). Table 3 provides the results from two independent experiments for 32 selected JCV siRNAs. The mean IC50 from these two independent experiments is shown. Several JCV siRNAs (AD-12622, AD-12677, AD-12709, AD-12710, AD-12722, AD-12724, AD-12728, AD-12763, AD-12767, AD-12768, AD-12769, AD-12771, AD-12774, AD-12775, AD-12777, AD-12781, AD-12784, AD-12795, AD-12813, AD-12821, AD-12823, AD-12824, AD-12825, AD-12827, AD-12829, AD-12842) were particularly potent in this experimental paradigm, and exhibited IC50 values between 70 pM and 1 nM.

TABLE 3
IC50s

| | Mean IC50 |
|---|---|
| Duplex name | [nM] |
| AD-12599 | 2.37 |
| AD-12622 | 0.57 |
| AD-12666 | 3.7 |
| AD-12677 | 0.49 |
| AD-12709 | 0.19 |
| AD-12710 | 0.47 |
| AD-12712 | 2.33 |
| AD-12722 | 0.12 |
| AD-12724 | 0.26 |
| AD-12728 | 0.8 |
| AD-12761 | 1.2 |
| AD-12763 | 0.95 |
| AD-12767 | 0.09 |
| AD-12768 | 0.19 |
| AD-12769 | 0.35 |
| AD-12771 | 0.35 |
| AD-12774 | 0.13 |
| AD-12775 | 0.18 |
| AD-12777 | 0.17 |
| AD-12778 | 12.65 |
| AD-12781 | 0.18 |
| AD-12784 | 0.44 |
| AD-12795 | 0.65 |
| AD-12813 | 0.2 |
| AD-12818 | 1.88 |
| AD-12821 | 0.07 |
| AD-12823 | 0.46 |
| AD-12824 | 0.25 |
| AD-12825 | 0.52 |
| AD-12827 | 0.15 |
| AD-12829 | 0.14 |
| AD-12842 | 0.44 |

### Example 3

### Screen of JCV siRNAs Against Live JC Virus in SVG-A Cells

Cells and Virus

SVG-A cells (human fetal glial cells transformed by SV40 T antigen) obtained from Walter Atwood at Brown University were cultured in Eagle's Minimum Essential Media (ATCC, Manassas, Va.) supplemented to contain 10% fetal bovine serum (FBS) (Omega Scientific, Tarzana, Calif.), Penicillin 100 U/ml, Streptomycin 100 ug/ml (Invitrogen, Carlsbad Calif.) at 37° C. in an atmosphere with 5% CO₂ in a humidified incubator (Heraeus HERAcell, Thermo Electron Corporation, Ashville, N.C.). The Mad-1-SVEΔ strain of JCV obtained from Walter Atwood at Brown University was used in all experiments; viral stocks were prepared using SVG-A cells according to standard published methods (Liu and Atwood, Propagation and assay of the JC Virus, Methods Mol. Biol. 2001; 165:9-17).

Prophylaxis Assay

SVG-A cells were seeded on glass coverslips in 6-well dishes 24 hours prior to transfection in the media described above minus antibiotics. Cells were transfected with the indicated concentration of siRNA (10 nM, 50 nM, or 100 nM) using Lipofectamine™ 2000 according to the manufacturer's instructions (Invitrogen, Carlsbad, Calif.). Twenty-four hours post-transfection cells were washed with media containing 2% FBS and then infected with a 1:25 dilution of JCV virus stock (Mad-1-SVEΔ strain) diluted in 2% FBS media. Cells were rocked every 15 minutes by hand several times to get equal virus binding across the entire coverslip for one hour and then additional 10% FBS media was added and the infection was allowed to proceed for 72 hours. Seventy two hours post-infection, cells were fixed in acetone and stained for late viral protein (VP1) by standard immunofluoresence methods using hybridoma supernatant PAB597 recognizing JCV VP1 (obtained from Walter Atwood at Brown University) with goat anti-mouse Alexa Fluor 488 secondary antibody (Invitrogen, Carlsbad, Calif.). Infected cells were scored by counting VP1-immunoreactive cells using a fluorescence microscope (Zeiss, Imager.Z1, Thornwood, N.J.) and data were expressed as the percentage of infected cells counted for the control coverslips transfected with Luciferase siRNA. Table 4 shows the results of the prophylaxis assays at different siRNA concentrations (10 nM, 50 nM or 100 nM). The VP1 siRNAs were the most potent as a group, followed by the T antigen siRNAs, with the VP2/3 siRNAs being the least potent. The VP1 siRNAs most effective in reducing virus were consistently AD-12622, AD-12728, AD-12795, and AD-12842. The most potent T antigen siRNA was AD-12813.

TABLE 4
Prophylaxis Assay

| | | Remaining Virus (% | | |
|---|---|---|---|---|
| | Targeted | of Luciferase | | |
| Duplex | JCV | Control) | | |
| Number | Transcript | 50 nM | 10 nM | 100 nM |
| AD-12599 | VP1 | 79.9 | ND | ND |
| AD-12709 | VP1 | 46.0 | ND | ND |
| AD-12710 | VP1 | 25.9 | ND | ND |
| AD-12784 | VP1 | 30.9 | ND | ND |
| AD-12712 | VP1 | 29.7 | ND | ND |
| AD-12724 | VP1 | 30.5 | 38.9 | 25.8 |
| AD-12622 | VP1 | 22.9 | 28.2 | 9.1 |
| AD-12728 | VP1 | 21.1 | 22.2 | ND |
| AD-12795 | VP1 | 13.6 | 16.9 | 8.5 |
| AD-12842 | VP1 | 16.0 | 23.4 | 12.7 |
| AD-12761 | VP1 | 26.4 | 52.3 | ND |
| AD-12818 | VP1 | 24.0 | 50.2 | 28.0 |
| AD-12666 | VP1 | 54.1 | ND | ND |
| AD-12763 | VP1 | 39.5 | ND | ND |
| AD-12722 | T Antigen | 43.6 | 82.1 | ND |
| AD-12813 | T Antigen | 21.5 | 48.8 | 19.4 |
| AD-12767 | T Antigen | 37.6 | 52.2 | 30.9 |
| AD-12821 | T Antigen | 33.0 | 51.2 | 30.8 |
| AD-12774 | T Antigen | 74.0 | 89.2 | ND |
| AD-12827 | T Antigen | 77.0 | 92.0 | ND |
| AD-12775 | T Antigen | 81.6 | 95.4 | ND |
| AD-12777 | T Antigen | 73.3 | 93.9 | ND |
| AD-12829 | T Antigen | 78.6 | 93.6 | ND |
| AD-12781 | T Antigen | 38.8 | 62.6 | 34.4 |
| AD-12768 | VP2/3 | 73.9 | 92.4 | ND |
| AD-12771 | VP2/3 | 51.6 | 83.6 | ND |
| AD-12824 | VP2/3 | 42.1 | 79.0 | 43.7 |
| AD-12769 | VP2/3 | 35.2 | 78.0 | 39.7 |
| AD-12823 | VP2/3 | 38.1 | 78.1 | 42.0 |
| AD-12677 | VP2/3 | 99.1 | 102.1 | ND |
| AD-12825 | VP2/3 | 100.8 | 99.1 | ND |

ND indicates no data.

Post-Infection Treatment Assay

SVG-A cells were seeded on glass coverslips in 6-well dishes 24 hours prior to infection in 10% FBS media. Cells were washed with media containing 2% FBS and then infected with a 1:25 dilution of JCV virus stock diluted in 2% FBS media. Cells were rocked by hand approximately 8-10 times to get equal virus binding across the entire coverslip every 15 minutes for one hour and then additional 10% FBS media was added. Twenty-four and forty-eight hours postinfection, cells were washed with 10% FBS media containing no antibiotics and then transfected with 50 nM of the indicated siRNA using Lipofectamine™ 2000 according to the manufacturer's instructions (Invitrogen, Carlsbad, Calif.). Seventy-two hours postinfection, cells were fixed in acetone and stained for late viral protein (VP1) by standard immunofluoresence methods using hybridoma supernatant PAB597 recognizing JCV VP1 (obtained from Walter Atwood at Brown University) with goat anti-mouse Alexa Fluor 488 secondary antibody (Molecular Probes, Eugene, Oreg.). Infected cells were scored by counting VP1-immunoreactive cells using a fluorescence microscope (Zeiss, Imager.Z1, Thornwood, N.J.) and data were expressed as the percentage of infected cells counted for control coverslips transfected with Luciferase siRNA. Table 5 shows the results of the post-infection treatment experiments. All of the siRNAs tested in the treatment assay showed significant antiviral activity against JCV, such that the remaining virus was significantly less than that in the luciferase siRNA control.

TABLE 5
Treatment Assay

| | Targeted JCV | Remaining Virus (% of |
|---|---|---|
| Duplex Number | Transcript | Luciferase Control) |
| AD-12724 | VP1 | 38.9 |
| AD-12622 | VP1 | 28.2 |
| AD-12795 | VP1 | 16.9 |
| AD-12842 | VP1 | 23.4 |
| AD-12818 | VP1 | ND |
| AD-12813 | T Antigen | 48 |
| AD-12767 | T Antigen | 56.9 |
| AD-12821 | T Antigen | 75.8 |
| AD-12781 | T Antigen | 75.8 |
| AD-12824 | VP2/3 | 60.4 |
| AD-12769 | VP2/3 | 70.7 |
| AD-12823 | VP2/3 | 72.4 |

ND indicates no data.

### Example 4

### Prophylaxis Administration of JCV siRNAs Inhibits the Production of Active Progeny JC Virus

SVG-A cells were seeded in 6-well dishes 24 hours prior to transfection in the media described above minus antibiotics. Cells were transfected with 10 nM of the indicated siRNA using Lipofectamine™ 2000 according to the manufacturer's instructions (Invitrogen, Carlsbad, Calif.). Twenty-four hours post-transfection cells were washed with media containing 2% FBS and then infected with a 1:25 dilution of JCV virus stock (Mad-1-SVEΔ strain) diluted in 2% FBS media. Cells were rocked every 15 minutes by hand several times to get equal virus binding across the entire coverslip for one hour and then additional 10% FBS media was added and the infection was allowed to proceed for 6 days. Six days post-infection, progeny virus was collected either by removal of overlay media from infected cells or by scraping cells and performing virus preparations. The virus preparations consisted of scraping cells into the supernatant media, vortexing, freeze-thawing the re-suspended cells 2 times with vortexing in between, then spinning down the cell debris and taking the supernatant. Fresh SVG-A cells seeded on glass coverslips were infected secondarily with virus collected by either method using the same procedure done with the initial infection to determine the amount of infectious virus produced by cells transfected with the various siRNAs. At 72 hours post-infection of coverslips, cells were fixed in acetone and stained for late viral protein (VP1) by standard immunofluoresence methods using hybridoma supernatant PAB597 recognizing JCV VP1 (obtained from Walter Atwood at Brown University) with goat anti-mouse Alexa Fluor 488 secondary antibody (Invitrogen, Carlsbad, Calif.). Infected cells were scored by counting VP1-immunoreactive cells using a fluorescence microscope (Zeiss, Imager.Z1, Thornwood, N.J.) and data were expressed as the percentage of infected cells counted for the control coverslips transfected with Luciferase siRNA. Table 6 shows the results for selected siRNAs, demonstrating the ability of prophylaxis siRNA treatment to inhibit active progeny virus production by either method of virus collection. Transfection with siRNAs targeting VP1 (AD-12622 and AD-12842) had the greatest effect on inhibiting the production of active progeny virus regardless of whether virus was collected from media or from infected cell preparations. The T antigen siRNA AD-12813 had the next strongest inhibitory effect, whereas the VP2/3 siRNAs AD-12824 and AD-12769 still showed some albeit a lesser ability to inhibit active progeny JCV production.

TABLE 6
Prophylaxis administration of JCV siRNAs inhibits the production
of active progeny JC virus capable of secondary infection

Remaining
Virus (% of
Luciferase
Control)

| Duplex | Targeted | | Virus |
|---|---|---|---|
| Name | Transcript | Media | Preparation |
| AD-12622 | VP1 | 30.8 | 24.9 |
| AD-12842 | VP1 | 33.3 | 26.9 |
| AD-12813 | T Antigen | 57.8 | 38.7 |
| AD-12824 | VP2/3 | 83.6 | 57.6 |
| AD-12769 | VP2/3 | 79.1 | 52.2 |

### Example 5

### Stability in Cerebrospinal Fluid (CSF) of Selected siRNAs Targeting JCV

Eleven selected JCV siRNAs were tested for stability at 5 uM over 48 h at 37° C. in human CSF, as well as in PBS for comparison. 30 μl of human cerebrospinal fluid (CSF) was mixed with 3 μl of 50 μM duplex (siRNA) solution (150 pmole/well) in a 96-well plate, sealed to avoid evaporation and incubated for the indicated time at 37° C. Incubation of the siRNA in 30 ul PBS for 48 h served as a control for non-specific degradation. Reactions were stopped by the addition of 4 ul proteinase K (20 mg/ml) and 25 ul of proteinase K buffer, and an incubation for 20′ at 42° C. Samples were then spin filtered through a 0.2 μm 96 well filter plate at 3000 rpm for 20′. Incubation wells were washed with 50 ul Millipore water twice and the combined washing solutions were spin filtered also.

Samples were analyzed by ion exchange HPLC under denaturing conditions. Samples were transferred to single autosampler vials. IEX-HPLC analysis was performed under the following conditions: Dionex DNAPac PA200 (4×250 mm analytical column), temperature of 45° C. (denaturing conditions by pH=11), flow rate of 1 ml/min, injection volume of 50 ul, and detection wavelength of 260 nm with 1 nm bandwidth (reference wavelength 600 nm). In addition, the gradient conditions were as follows with HPLC Eluent A: 20 mM Na₃PO₄ in 10% ACN; pH=11 and HPLC Eluent B: 1 M NaBr in HPLC Eluent A:

| Time | % A | % B |
|---|---|---|
| 0.00 min | 75 | 25 |
| 1.00 min | 75 | 25 |
| 19.0 min | 38 | 62 |
| 19.5 min | 0 | 100 |
| 21.5 min | 0 | 100 |
| 22.0 min | 75 | 25 |
| 24.0 min | 75 | 25 |

Under the above denaturing IEX-HPLC conditions, the duplexes eluted as two separated single strands. All chromatograms were integrated automatically by the Dionex Chromeleon 6.60 HPLC software, and were adjusted manually as necessary. The area under the peak for each strand was calculated and the %-values for each intact full length product (FLP) for each time points were calculated by the following equation:

%-FLP_{(s/as; t=x)}=(PeakArea_{(s/as); t=x}/PeakArea_{(s/as); t=0min})*100%

All values were normalized to FLP at t=0 min. Table 7 provides the results after 48 hours of incubation in human CSF at 37° C. At least 75% of both antisense and sense strands of ten JCV siRNAs (AD-12622, AD-12724, AD-12767, AD-12769, AD-12795, AD-12813, AD-12818, AD-12823, AD-12824, AD-12842) were recovered, demonstrating that these siRNAs are highly stable in human CSF at 37° C. For AD-12821, 59% of the antisense and 97% of the sense strand was recovered after 48 h of incubation in human CSF at 37° C., showing that this siRNA has a half-life of greather than 48 h in human CSF at 37° C.

TABLE 7
Stability in human CSF

| | |
|---|---|
| | % full length |
| | material after |
| Duplex | 48 hours |

| name | antisense | sense |
|---|---|---|
| AD-12622 | 93 | 105 |
| AD-12724 | 90 | 106 |
| AD-12767 | 85 | 104 |
| AD-12769 | 100 | 104 |
| AD-12795 | 86 | 109 |
| AD-12813 | 94 | 98 |
| AD-12818 | 75 | 99 |
| AD-12821 | 59 | 97 |
| AD-12823 | 98 | 98 |
| AD-12824 | 84 | 98 |
| AD-12842 | 87 | 102 |

### Example 6

### In Vivo Down-Modulation of Endogenous CNP mRNA Levels by CNS Administration of Unconjugated CNP dsRNAs in Rats

Progressive multifocal leukoencephalopathy (PML) is a rapid and fatal demyelinating disease of the CNS that can occur in immunocompromised individuals. The human polyomavirus JCV has been identified as a causative agent for PML. The primary cell type infected by JCV is the oligodendrocyte. siRNAs targeting an endogenous oligodendrocyte gene (cyclic nucleotide phosphodiesterase; CNP) were used to show successful delivery of siRNAs in vivo into oligodendrocytes of normal rats. siRNAs targeting CNP and formulated in saline were infused into the rat corpus callosum where they robustly silenced the CNP gene. This silencing was durable, dose dependent, and mediated by an RNAi mechanism.

Male Sprague-Dawley rats were used in all studies (˜300 g body weight, Charles River Laboratory). Animal maintenance and surgical procedures were conducted in strict compliance with protocols approved by the Institutional Animal Care and Use Committee. Rats were anesthetized with a mixture of 0.5 ml/kg of ketamine (150 mg)/xylazine (30 mg)/acepromazine (5 mg), and placed into a stereotaxic frame (Benchmark™ Digital Stereotaxic, myNeurol.ab). Aseptic techniques were used throughout the surgical procedure. A burr hole was drilled in the rat skull, and a 30 gauge osmotic pump infusion cannula (Plastics One) was implanted into the right hemisphere, targeting the corpus callosum (stereotaxic coordinates AP 0.7, ML 2.2 and DV 3.0 relative to bregma; incisor bar 3.3 mm below the interaural line). Osmotic pumps (10 μl/hr flow rate, Alzet) containing PBS, control siRNA targeting luciferase AD-1955 (AS AL-3374: 5′-UCGAAGuACUcAGCGuAAGTsT-3′ (SEQ ID NO:2093), S AL-3372: 5′-cuuAcGcuGAGuAcuucGATsT-3′ (SEQ ID NO:2094)), or CNP siRNAs; AD-12436 (AS AL-20069: 5′-UCUCuAAGAGGUcAAGGCCTsT-3′ (SEQ ID NO:2095), S AL-20068: 5′-GGccuuGAccucuuAGAGATsT-3′ (SEQ ID NO:2096)), AD-12449 (AS AL-20095: 5′-cAAGGAAuAGAGCUUGCCCTsT-3′ (SEQ ID NO:2097), S AL-20094: 5′-GGGcAAGcucuAuuccuuGTsT-3′ (SEQ ID NO:2098)), AD-12441 (AS AL-20079: 5′-AUCUCuAAGAGGUcAAGGCTsT-3′ (SEQ ID NO:2099), S AL-20078: 5′-GccuuGAccucuuAGAGAuTsT-3′ (SEQ ID NO:2100)), AD-12438 (AS AL-20073: 5′-AAUCUCuAAGAGGUcAAGGTsT-3′ (SEQ ID NO:2101), S AL-20072: 5′-ccuuGAccucuuAGAGAuuTsT-3′ (SEQ ID NO:2102)) (see also Table 19) were primed in 0.9% saline overnight at 37° C. according to the manufacturer's instructions, and then connected to the cannula and implanted subcutaneously. All siRNAs were formulated in PBS. In the above sequences, lower case indicates 2′-O-Me modified nucleotides, and “s” indicates phosphorothioate linkages. siRNAs were generated by annealing equimolar amounts of complementary sense and antisense strands.

After 3-7 days of infusion, rats were euthanized and brains removed. Twelve coronal slices, each 1 mm thick, through the rat brain from anterior to posterior were obtained using a brain matrix (Braintree Scientific). The corpus callosum ipsilateral to the infusion was dissected from each slice and snap-frozen in liquid nitrogen for later mRNA measurement. The QuantiGene assay (Panomics) was used to quantify levels of CNP and myelin basic protein (MBP) mRNAs in rat corpus callosum after administration of siRNAs targeting CNP. Tissue lysates were directly used for CNP and MBP quantification, according to the manufacturer's instructions. CNP mRNA levels were normalized to MBP mRNA levels, and then further normalized to either PBS or control siRNA targeting luciferase (AD-1955).

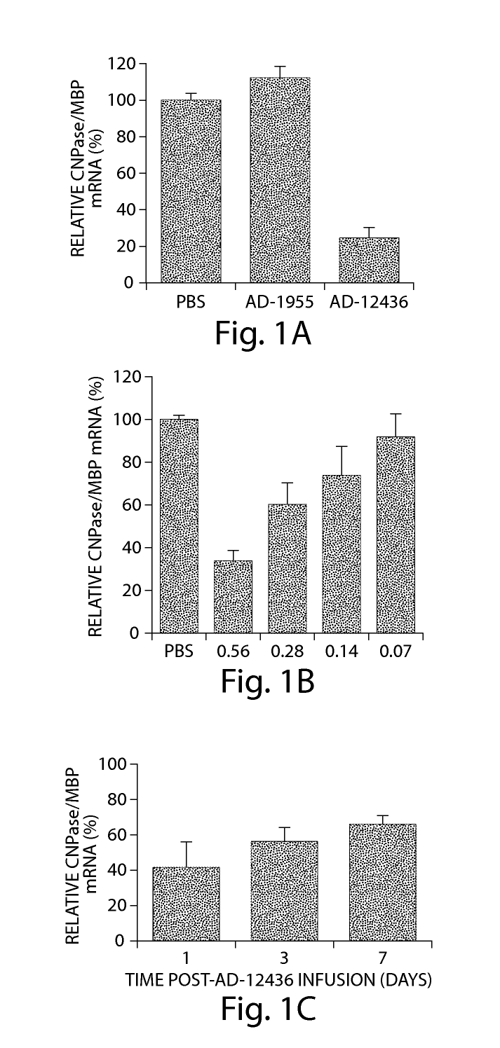

Infusion into the rat corpus callosum of CNP siRNA AD-12436 formulated simply in PBS, resulted in a 75% reduction of CNP mRNA relative to MBP mRNA (used for normalization) detected by branched DNA analysis (FIG. 1A). An siRNA targeting the non-mammalian gene, luciferase (AD-1955), had no effect on CNP mRNA levels, as expected (FIG. 1A). The dose-response for CNP reduction demonstrated that maximal down-modulation was achieved with 0.56 mM CNP siRNA AD-12436 (FIG. 1B), whereas the threshold concentration for silencing was between 70 and 140 uM. CNP siRNAs AD-12449, AD-12441, and AD-12438 also significantly silenced the CNP mRNA by 56%, 59%, and 40% respectively after 7 days of continuous infusion at 0.56 mM.

RNAi effects have been reported in peripheral organs to persist for 1-4 weeks following termination of siRNA administration (Bartlett, D. W. and Davis, M. E., Biotechnol. Bioeng. (2007), 97: 909-21). To evaluate the durability of CNP silencing, AD-12436 was infused into the rat corpus callosum for 3 days, and then after recovery periods of 1, 3 or 7 days, CNP mRNA levels were evaluated. After a recovery period of 7 days, CNP mRNA levels continued to be suppressed, although the magnitude of down-modulation after a recovery period of 1 or 3 days was greater (FIG. 1C).

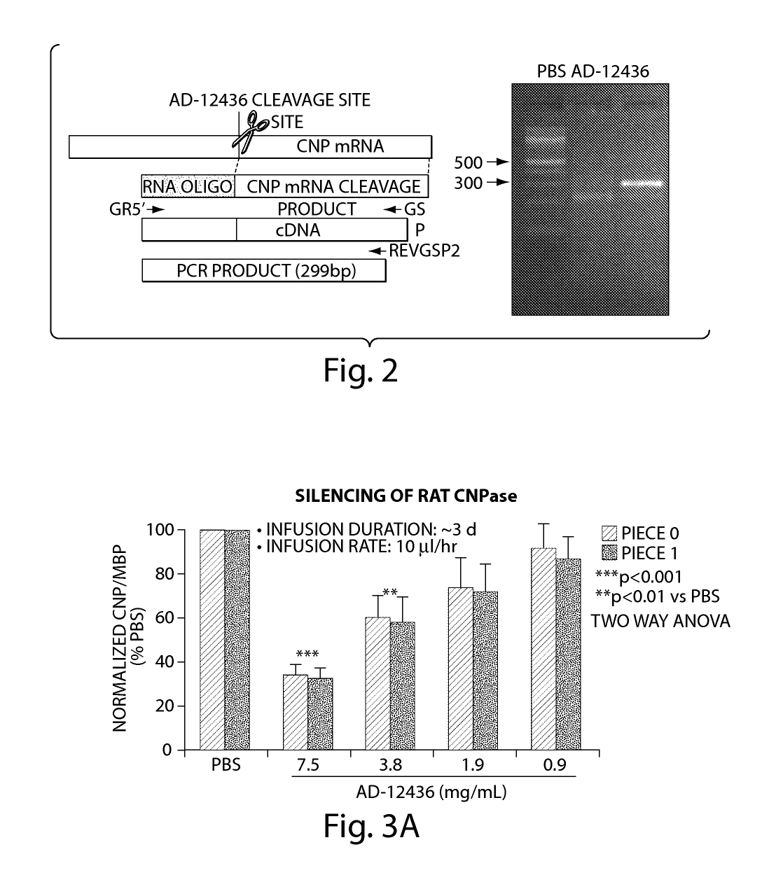

To confirm that silencing of the CNP mRNA was occurring by an RNAi mechanism 5′RACE analysis was performed. Total RNA extracted from corpus callosum tissue pooled from rats infused with AD-12436 or PBS (control) was ligated to GeneRacer Oligo (Invitrogen) without prior processing. Ligated mRNA was reverse transcribed into cDNA using a CNP specific primer 5′-CCACCTGCCTGTGTTGAGCTGAGTGTT-3′ (SEQ ID NO:2103). To detect cleavage product, PCR was performed using Platinum Taq Polymerase (Invitrogen) with the GeneRacer 5′ primer (Invitrogen) and CNP specific primer: 5′-CCACAGCGGTGGCACAGTGGCGTGAA-3′ (SEQ ID NO:2104). Amplification fragments were resolved on a 2% agarose gel and excised bands were cloned into pCR4-TOPO vector (Invitrogen) and confirmed by sequencing. We detected a band of the predicted size in tissue from AD-12436-treated but not control animals (FIG. 2). Sequencing of the excised band demonstrated that cleavage occurred at the predicted site within the CNP mRNA target region. This result suggested that the reduction in CNP mRNA levels seen following AD-12436 infusion was occurring via an RNAi mechanism.

In summary, dose dependent silencing of CNP that was durable for up to 7 days was achieved and was confirmed to be mediated by an RNAi mechanism. In view of these results, an siRNA targeting JCV offers significant promise for the effective inhibition of JCV replication in oligodendrocytes and for the treatment of PML.

These findings not only have broad implications for research studies, enabling the use of siRNAs to study the role of different molecular targets in oligodendrocytes in vivo, but also indicates that direct CNS administration of siRNA has clinical application for inhibiting pathogenic molecules in oligodendrocytes. These applications include demyelinating diseases such as PML, multiple sclerosis and leukodystrophy, as well as neurological disorders and injuries where axonal regeneration involving neuron-oligodendrocyte interactions and re-myelination represent therapeutic strategies.

### Example 7

### In Vivo Down-Modulation of Endogenous CNPase with AD-12436

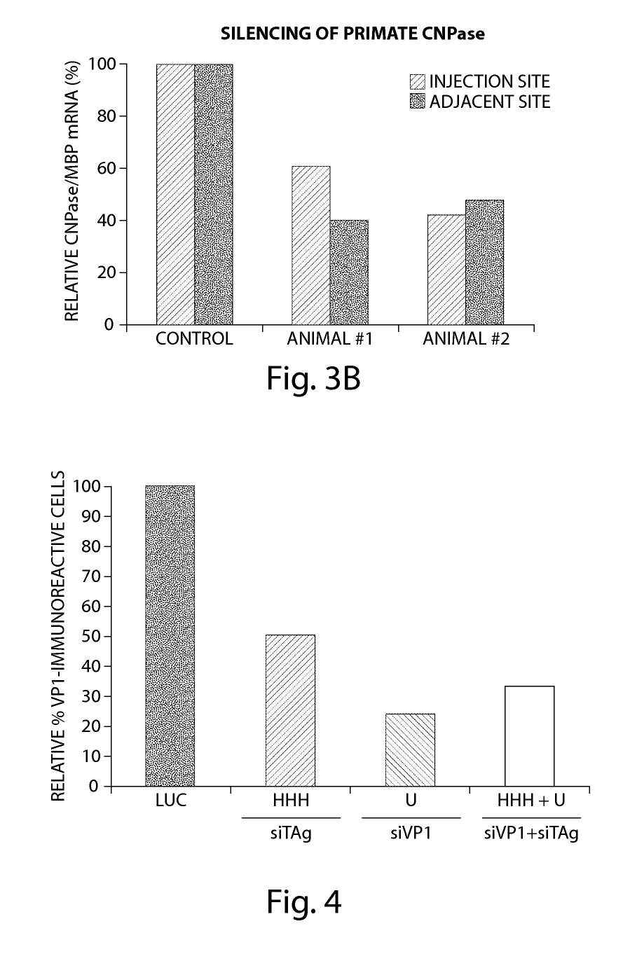

dsRNA AD-12436 was administered to rats and primates as shown in FIGS. 3A and 3B, respectively.

Experiments in rats demonstrated that silencing by siCNP is dose-dependent (n=6 per group). Intraparenchymal infusion of siCNP into the corpus callosum of rats for three days at 7.5 mg/ml was performed (stereotaxic coordinates AP 0.7, ML 2.2 and DV 3.0 relative to bregma). Following animal sacrifice, 1 mm coronal sections of brain were cut from anterior to posterior, then corpus callosum tissue pieces were dissected for evaluation of CNP mRNA knockdown. Exemplary data is shown in FIG. 3A.

siCNP also demonstrated silencing of the oligodendrocyte target, CNP, in non-human primates (FIG. 3B). Intraparenchymal infusion of siCNP into the corona radiata resulted in robust silencing of CNP mRNA at the infusion site and in adjacent white matter tissue punches in both animals examined (#1, #2), compared to a naïve animal (Control).

### Example 8

### In Vivo Down-Modulation of Endogenous CNPase with AD-3222 and AD-3178

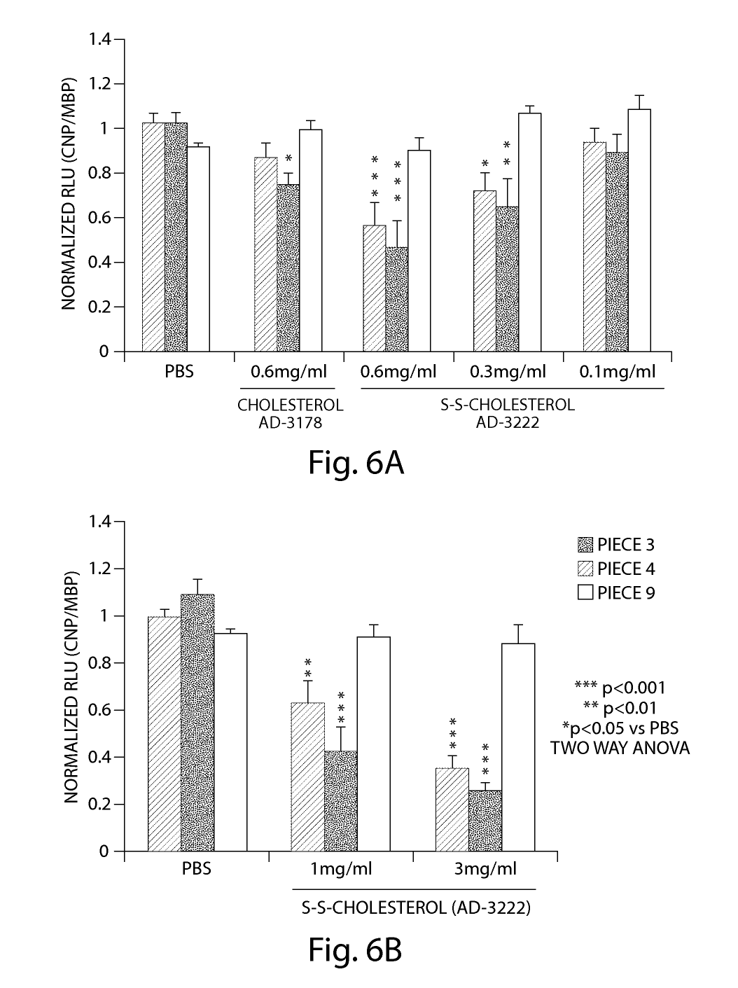

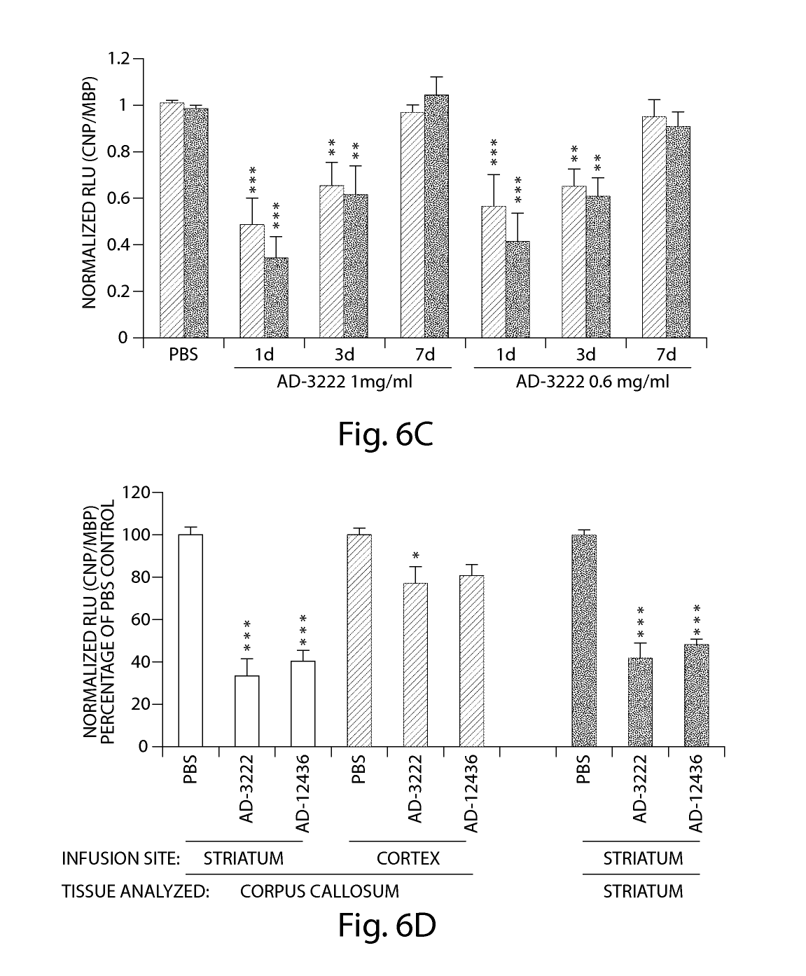

dsRNA AD-3222 and AD 3178 were administered to rats as shown in FIGS. 6A-6D.

Experiments in rats demonstrated that silencing by siCNP is dose-dependent (n=6 per group). Intraparenchymal, intrastriatial or intracortical convection enhanced delivery (CED) of siCNP into the corpus callosum or cortex of rats for three or seven days at a infusion rate of 10 μL/hr. Following animal sacrifice, 1 mm coronal sections of brain were cut from anterior to posterior, then corpus callosum tissue pieces were dissected for evaluation of CNP mRNA knockdown. Exemplary data is shown in FIGS. 6A-6D. According to FIGS. 6A and 6B, ˜26% maximum knockdown after CED of 0.6 mg/ml (0.43 mg) CNP siRNA AD-3178 into corpus callosum and ˜55% maximum knockdown after CED of 0.6 mg/ml (0.43 mg) CNP siRNA AD-3222 into corpus callosum. ˜64% maximum knockdown after CED of 1 mg/ml (0.72 mg) was observed with AD-3222 CNP siRNA into corpus callosum. ˜78% maximum knockdown after CED of 3 mg/ml (2.16 mg) AD-3222 CNP siRNA into corpus callosum. In addition, it was observed that S—S-cholesterol conjugated CNPase siRNA (AD-3222) produces better silencing of CNPase mRNA than regular cholesterol conjugated siRNA (AD-3178) (˜55% maximum knockdown at 0.6 mg/ml in S—S-cholesterol conjugated group and ˜26% maximum knockdown at 0.6 mg/ml in cholesterol conjugated group).

In a durability study of CNPase mRNA silencing after intraparenchymal CED at 1, 3 and 7 day washout (FIG. 6C), ˜68% (at 1 mg/ml) and ˜59% (at 0.6 mg/ml) maximum knockdown after 1 day washout, ˜38% (at 1 mg/ml) and ˜39% (at 0.6 mg/ml) maximum knockdown after 3 day washout. Therefore, durability of silencing induced by AD-3222 at either 1 mg/ml or 0.6 mg/ml is >3 days.

CNPase silencing with intrastriatal or intracortical CED infusion data (FIG. 6D) showed ˜67% (AD-3222 at 1 mg/ml) and ˜60% (AD-12436 at 7.5 mg/ml) maximum knockdown in the corpus callosum after 7 day intrastriatal infusion, ˜22% (AD-3222) and ˜19% (AD-12436) maximum knockdown in the corpus collosum after 7 day intracortical infusion, and ˜59% (AD-3222) and ˜53% (AD-12436) knockdown in the striatum after 7 day intrastriatal infusion. Thus, intrastriatial infusion of either unconjugated or S—S-Chol-conjugated CNPase siRNA (AD-3222) produces significant silencing in the corpus collosum (˜67% maximum knockdown at 1 mg/ml in S—S-cholesterol conjugated group (AD-3222), and ˜60% maximum knockdown at 7.5 mg/ml in unconjugated group (AD-12436)). In addition, intrastriatial infusion of either unconjugated or S—S-Chol-conjugated CNPase siRNA produces significant silencing in the striatum (˜59% maximum knockdown at 1 mg/ml in S—S-cholesterol conjugated group (AD-3222), and ˜53% maximum knockdown at 7.5 mg/ml in unconjugated group (AD-12436)).

### Example 9

### In Vivo Down-Modulation of Endogenous CNPase with AD-3569 and AD-3181

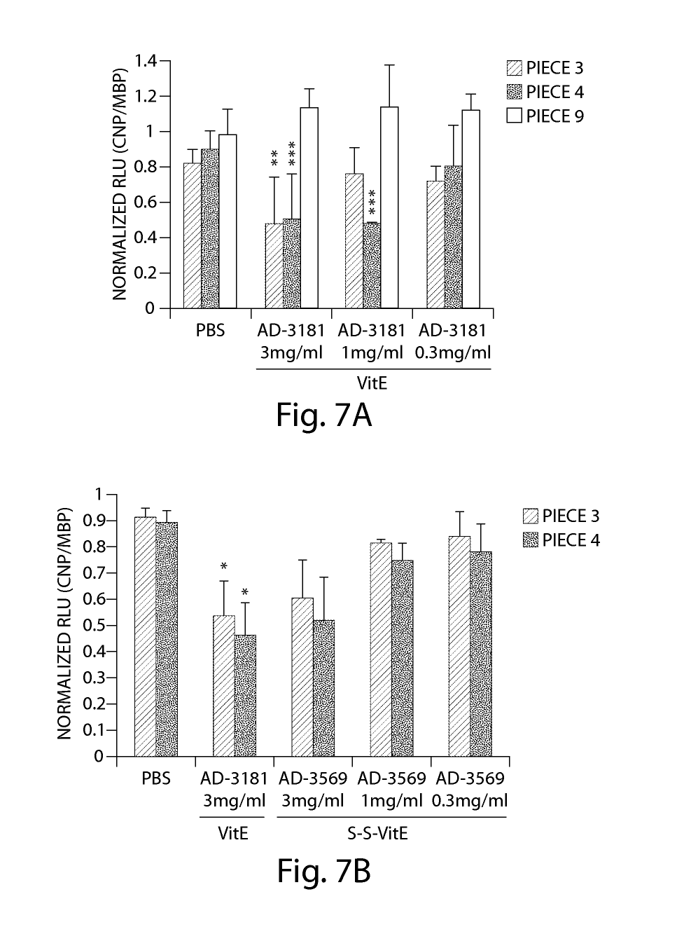

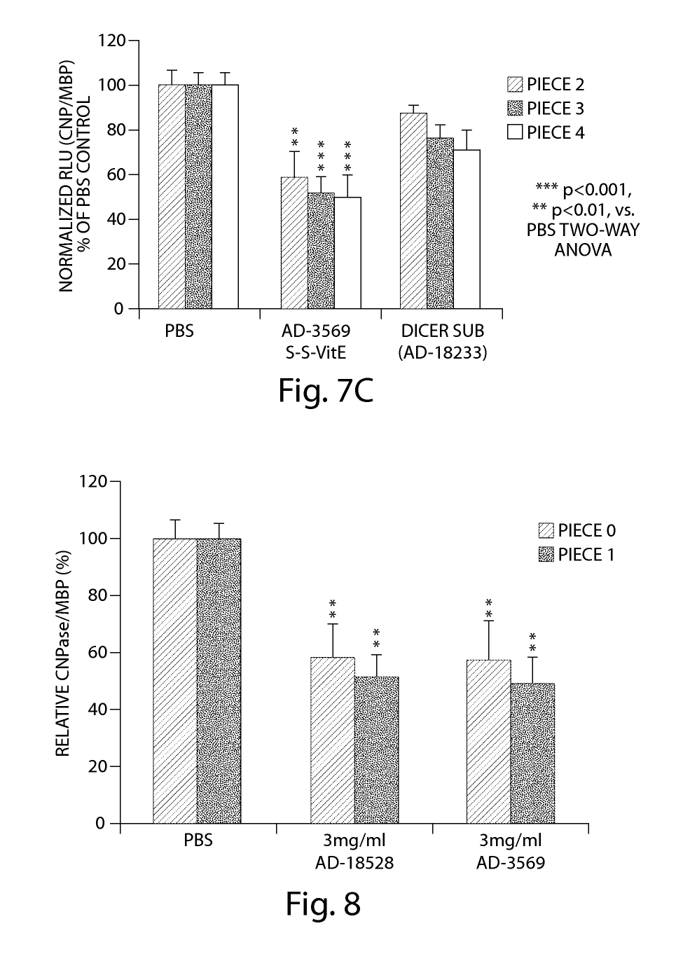

dsRNA AD-3569, AD-3181 and dicer substrate AD-18233 were administered to rats as shown in FIGS. 7A-7C.

Experiments in rats demonstrated that silencing by siCNP is dose-dependent (n=4-6 per group). Intraparenchymal convection enhanced delivery (CED) of siCNP into the corpus callosum or cortex of rats for three or seven days at a infusion rate of 10 μL/hr. Following animal sacrifice, 1 mm coronal sections of brain were cut from anterior to posterior, then corpus callosum tissue pieces were dissected for evaluation of CNP mRNA knockdown. Exemplary data is shown in FIGS. 7A-7C. According to FIG. 7A and FIG. 7B, ˜48% (AD-3181) and ˜41% (AD-3569) maximum knockdown in the corpus callosum at 3 mg/ml and ˜43% (AD-3181) and ˜16% (AD-3569) knockdown at 1 mg/ml. As shown in FIG. 7C, AD-3569 produces better silencing of CNPase mRNA than dicer substrate AD-18233 (˜50% maximum knockdown after CED of 3 mg/ml (2.16 mg) S—S-VitE conjugated CNP siRNA (AD-3569) into corpus callosum Versus ˜30% maximum knockdown after CED of 3 mg/ml (2.16 mg) Dicer CNP siRNA (AD-18233) into corpus callosum.

### Example 10

### Silencing Effect of Vitamin E Conjugates with Cleavable Disulfide Linkers

Vitamin E conjugated CNPase siRNA with non-cleavable linker (siCNP-VitE-1, 3 mg/ml), Vitamin E conjugate with disulfide linkers (siCNP-VitE-2, 0.3-3 mg/ml; siCNP-VitE-3, 3 mg/ml) or PBS was infused at 10 μl/hr over 3 days into the rat corpus callosum using an Alzet osmotic pump (2ML1). The QuantiGene assay was used to quantify levels of CNPase mRNA, normalized to myelin basic protein (MBP) mRNA in rat corpus callosum after administration of CNPase siRNA. Signals were further normalized to the PBS control group. N=4-5 per group.

At 3 mg/ml, the knockdown was comparable for the siCNP-VitE-1 with non-cleavable linker, AD-3569 and AD-18528 with cleavable disulfide linkers; the knockdown was also comparable for both disulfide constructs.

### Example 11

### VP1 siRNAs are More Potent Alone than when Combined at Half Doses with T Antigen siRNAs

A VP1 or T antigen siRNA either alone at 10 nM or in combination at 5 nM each were transfected into SVG cells. Twenty four hours post-transfection, cells were infected with JCV, and the infection was allowed to proceed for 72 hours. Cells were then fixed and stained for VP1 expression, and then scored by counting using a fluorescent microscope. The data as shown in FIG. 4 is expressed as the percentage of control infected cells transfected with Luciferase siRNAs. The silencing effect in combination is largely driven by the more potent VP1 siRNA.

### Example 12

### JCV siRNAs do not Induce IFN-α or TNF-α Release in a Human PBMC Assay

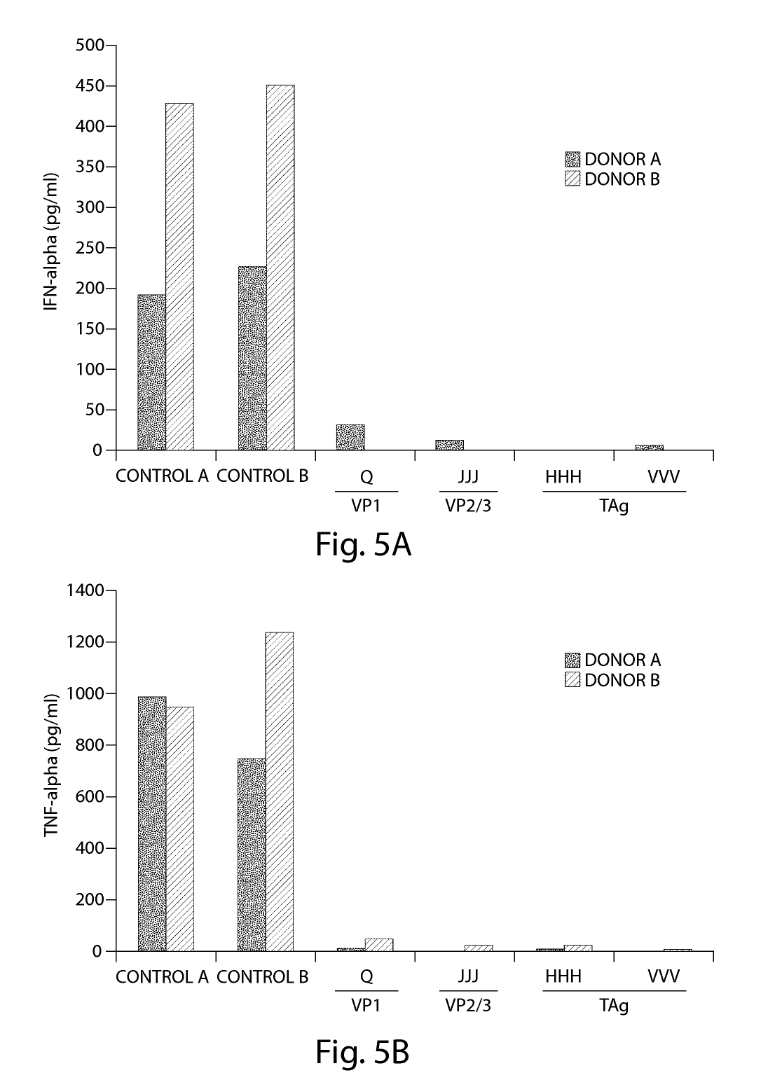

Human blood was obtained from two anonymous donors through a blood bank. Peripheral blood mononuclear cells (PBMCs) were isolated and transfected with 130 nM siRNA using GenePorter2 for IFN-α analysis and DOTAP for TNF-α analysis. Tissue culture supernatants were collected 24 h post-transfection, and cytokine levels were determined by ELISA. The data is summarized in FIGS. 5A and 5B. Control A and Control B represent two unrelated oligonucleotides that serve as positive controls.

TABLE 8

JCV Gene Walk. siRNAs targeting >95% of all strains
(>=369 out of 388). Human specific pan-JCV: 208 siRNAs;
all siRNAs double overhang design, dTdT, no modifications

| | | SEQ | | SEQ | |
|---|---|---|---|---|---|
| Position in | Duplex | ID | Sequence | ID | Sequence |
| Consensus | Name | NO: | (5′--> 3′) | NO: | (5′--> 3′) |
| 1533-1551 | AD-14742 | 515 | CUUAUAAGAGGAGGAGUAGTT | 516 | CUACUCCUCCUCUUAUAAGTT |
| 1703-1721 | AD-14743 | 517 | CAUGCUUCCUUGUUACAGUTT | 518 | ACUGUAACAAGGAAGCAUGTT |
| 1439-1457 | AD-14744 | 519 | UACGGGACUGUAACACCUGTT | 520 | CAGGUGUUACAGUCCCGUATT |
| 1705-1723 | AD-14745 | 521 | UGCUUCCUUGUUACAGUGUTT | 522 | ACACUGUAACAAGGAAGCATT |
| 2064-2082 | AD-14746 | 523 | CCUGUUGAAUGUUGGGUUCTT | 524 | GAACCCAACAUUCAACAGGTT |
| 2067-2085 | AD-14747 | 525 | GUUGAAUGUUGGGUUCCUGTT | 526 | CAGGAACCCAACAUUCAACTT |
| 2071-2089 | AD-14748 | 527 | AAUGUUGGGUUCCUGAUCCTT | 528 | GGAUCAGGAACCCAACAUUTT |
| 2121-2139 | AD-14749 | 529 | ACACUAACAGGAGGAGAAATT | 530 | UUUCUCCUCCUGUUAGUGUTT |
| 1535-1553 | AD-14750 | 531 | UAUAAGAGGAGGAGUAGAATT | 532 | UUCUACUCCUCCUCUUAUATT |
| 1536-1554 | AD-14751 | 533 | AUAAGAGGAGGAGUAGAAGTT | 534 | CUUCUACUCCUCCUCUUAUTT |
| 1445-1463 | AD-14752 | 535 | ACUGUAACACCUGCUCUUGTT | 536 | CAAGAGCAGGUGUUACAGUTT |
| 1700-1718 | AD-14753 | 537 | GGACAUGCUUCCUUGUUACTT | 538 | GUAACAAGGAAGCAUGUCCTT |
| 1702-1720 | AD-14754 | 539 | ACAUGCUUCCUUGUUACAGTT | 540 | CUGUAACAAGGAAGCAUGUTT |
| 1704-1722 | AD-14755 | 541 | AUGCUUCCUUGUUACAGUGTT | 542 | CACUGUAACAAGGAAGCAUTT |
| 2065-2083 | AD-14756 | 543 | CUGUUGAAUGUUGGGUUCCTT | 544 | GGAACCCAACAUUCAACAGTT |
| 2070-2088 | AD-14757 | 545 | GAAUGUUGGGUUCCUGAUCTT | 546 | GAUCAGGAACCCAACAUUCTT |
| 1441-1459 | AD-14758 | 547 | CGGGACUGUAACACCUGCUTT | 548 | AGCAGGUGUUACAGUCCCGTT |
| 1443-1461 | AD-14759 | 549 | GGACUGUAACACCUGCUCUTT | 550 | AGAGCAGGUGUUACAGUCCTT |
| 1444-1462 | AD-14760 | 551 | GACUGUAACACCUGCUCUUTT | 552 | AAGAGCAGGUGUUACAGUCTT |
| 1609-1627 | AD-14761 | 553 | CUCCAGAAAUGGGUGACCCTT | 554 | GGGUCACCCAUUUCUGGAGTT |
| 1537-1555 | AD-14762 | 555 | UAAGAGGAGGAGUAGAAGUTT | 556 | ACUUCUACUCCUCCUCUUATT |
| 629-647 | AD-14763 | 557 | GAGGCUGCUGCUACUAUAGTT | 558 | CUAUAGUAGCAGCAGCCUCTT |
| 656-674 | AD-14764 | 559 | AUUGCAUCCCUUGCUACUGTT | 560 | CAGUAGCAAGGGAUGCAAUTT |
| 658-676 | AD-14765 | 561 | UGCAUCCCUUGCUACUGUATT | 562 | UACAGUAGCAAGGGAUGCATT |
| 517-535 | AD-14766 | 563 | UUGUGUUUUCAGGUUCAUGTT | 564 | CAUGAACCUGAAAACACAATT |
| 559-577 | AD-14767 | 565 | GGACCUAGUUGCUACUGUUTT | 566 | AACAGUAGCAACUAGGUCCTT |
| 591-609 | AD-14768 | 567 | CUGCCACAGGAUUUUCAGUTT | 568 | ACUGAAAAUCCUGUGGCAGTT |
| 638-656 | AD-14769 | 569 | GCUACUAUAGAAGUUGAAATT | 570 | UUUCAACUUCUAUAGUAGCTT |
| 655-673 | AD-14770 | 571 | AAUUGCAUCCCUUGCUACUTT | 572 | AGUAGCAAGGGAUGCAAUUTT |
| 561-579 | AD-14771 | 573 | ACCUAGUUGCUACUGUUUCTT | 574 | GAAACAGUAGCAACUAGGUTT |
| 639-657 | AD-14772 | 575 | CUACUAUAGAAGUUGAAAUTT | 576 | AUUUCAACUUCUAUAGUAGTT |
| 715-733 | AD-14773 | 577 | AGGCCUUACUCCUGAAACATT | 578 | UGUUUCAGGAGUAAGGCCUTT |
| 716-734 | AD-14774 | 579 | GGCCUUACUCCUGAAACAUTT | 580 | AUGUUUCAGGAGUAAGGCCTT |
| 326-344 | AD-14775 | 581 | GUAAAACCUGGAGUGGAACTT | 582 | GUUCCACUCCAGGUUUUACTT |
| 518-536 | AD-14776 | 583 | UGUGUUUUCAGGUUCAUGGTT | 584 | CCAUGAACCUGAAAACACATT |
| 520-538 | AD-14777 | 585 | UGUUUUCAGGUUCAUGGGUTT | 586 | ACCCAUGAACCUGAAAACATT |
| 661-679 | AD-14778 | 587 | AUCCCUUGCUACUGUAGAGTT | 588 | CUCUACAGUAGCAAGGGAUTT |
| 560-578 | AD-14779 | 589 | GACCUAGUUGCUACUGUUUTT | 590 | AAACAGUAGCAACUAGGUCTT |
| 681-699 | AD-14780 | 591 | GGAUUACAAGUACCUCUGATT | 592 | UCAGAGGUACUUGUAAUCCTT |
| 714-732 | AD-14781 | 593 | UAGGCCUUACUCCUGAAACTT | 594 | GUUUCAGGAGUAAGGCCUATT |
| 377-395 | AD-14782 | 595 | UGUUAGAAUUUUUGCUGGATT | 596 | UCCAGCAAAAAUUCUAACATT |
| 589-607 | AD-14783 | 597 | UGCUGCCACAGGAUUUUCATT | 598 | UGAAAAUCCUGUGGCAGCATT |
| 594-612 | AD-14784 | 599 | CCACAGGAUUUUCAGUAGCTT | 600 | GCUACUGAAAAUCCUGUGGTT |
| 648-666 | AD-14785 | 601 | AAGUUGAAAUUGCAUCCCUTT | 602 | AGGGAUGCAAUUUCAACUUTT |
| 649-667 | AD-14786 | 603 | AGUUGAAAUUGCAUCCCUUTT | 604 | AAGGGAUGCAAUUUCAACUTT |
| 587-605 | AD-14787 | 605 | GCUGCUGCCACAGGAUUUUTT | 606 | AAAAUCCUGUGGCAGCAGCTT |
| 325-343 | AD-14788 | 607 | AGUAAAACCUGGAGUGGAATT | 608 | UUCCACUCCAGGUUUUACUTT |
| 515-533 | AD-14789 | 609 | UUUUGUGUUUUCAGGUUCATT | 610 | UGAACCUGAAAACACAAAATT |
| 516-534 | AD-14790 | 611 | UUUGUGUUUUCAGGUUCAUTT | 612 | AUGAACCUGAAAACACAAATT |
| 519-537 | AD-14791 | 613 | GUGUUUUCAGGUUCAUGGGTT | 614 | CCCAUGAACCUGAAAACACTT |
| 521-539 | AD-14792 | 615 | GUUUUCAGGUUCAUGGGUGTT | 616 | CACCCAUGAACCUGAAAACTT |
| 522-540 | AD-14793 | 617 | UUUUCAGGUUCAUGGGUGCTT | 618 | GCACCCAUGAACCUGAAAATT |
| 523-541 | AD-14794 | 619 | UUUCAGGUUCAUGGGUGCCTT | 620 | GGCACCCAUGAACCUGAAATT |
| 616-634 | AD-14795 | 621 | AAUUGCUGCUGGAGAGGCUTT | 622 | AGCCUCUCCAGCAGCAAUUTT |
| 657-675 | AD-14796 | 623 | UUGCAUCCCUUGCUACUGUTT | 624 | ACAGUAGCAAGGGAUGCAATT |
| 761-779 | AD-14797 | 625 | GCUGUAGCUGGGUUUGCUGTT | 626 | CAGCAAACCCAGCUACAGCTT |
| 645-663 | AD-14798 | 627 | UAGAAGUUGAAAUUGCAUCTT | 628 | GAUGCAAUUUCAACUUCUATT |
| 647-665 | AD-14799 | 629 | GAAGUUGAAAUUGCAUCCCTT | 630 | GGGAUGCAAUUUCAACUUCTT |
| 660-678 | AD-14800 | 631 | CAUCCCUUGCUACUGUAGATT | 632 | UCUACAGUAGCAAGGGAUGTT |
| 324-342 | AD-14801 | 633 | UAGUAAAACCUGGAGUGGATT | 634 | UCCACUCCAGGUUUUACUATT |
| 372-390 | AD-14802 | 635 | UUUUUUGUUAGAAUUUUUGTT | 636 | CAAAAAUUCUAACAAAAAATT |
| 640-658 | AD-14803 | 637 | UACUAUAGAAGUUGAAAUUTT | 638 | AAUUUCAACUUCUAUAGUATT |
| 562-580 | AD-14804 | 639 | CCUAGUUGCUACUGUUUCUTT | 640 | AGAAACAGUAGCAACUAGGTT |
| 563-581 | AD-14805 | 641 | CUAGUUGCUACUGUUUCUGTT | 642 | CAGAAACAGUAGCAACUAGTT |
| 566-584 | AD-14806 | 643 | GUUGCUACUGUUUCUGAGGTT | 644 | CCUCAGAAACAGUAGCAACTT |
| 625-643 | AD-14807 | 645 | UGGAGAGGCUGCUGCUACUTT | 646 | AGUAGCAGCAGCCUCUCCATT |
| 627-645 | AD-14808 | 647 | GAGAGGCUGCUGCUACUAUTT | 648 | AUAGUAGCAGCAGCCUCUCTT |
| 628-646 | AD-14809 | 649 | AGAGGCUGCUGCUACUAUATT | 650 | UAUAGUAGCAGCAGCCUCUTT |
| 632-650 | AD-14810 | 651 | GCUGCUGCUACUAUAGAAGTT | 652 | CUUCUAUAGUAGCAGCAGCTT |
| 513-531 | AD-14811 | 653 | UUUUUUGUGUUUUCAGGUUTT | 654 | AACCUGAAAACACAAAAAATT |
| 641-659 | AD-14812 | 655 | ACUAUAGAAGUUGAAAUUGTT | 656 | CAAUUUCAACUUCUAUAGUTT |
| 323-341 | AD-14813 | 657 | UUAGUAAAACCUGGAGUGGTT | 658 | CCACUCCAGGUUUUACUAATT |
| 717-735 | AD-14814 | 659 | GCCUUACUCCUGAAACAUATT | 660 | UAUGUUUCAGGAGUAAGGCTT |
| 646-664 | AD-14815 | 661 | AGAAGUUGAAAUUGCAUCCTT | 662 | GGAUGCAAUUUCAACUUCUTT |
| 592-610 | AD-14816 | 663 | UGCCACAGGAUUUUCAGUATT | 664 | UACUGAAAAUCCUGUGGCATT |
| 590-608 | AD-14817 | 665 | GCUGCCACAGGAUUUUCAGTT | 666 | CUGAAAAUCCUGUGGCAGCTT |
| 526-544 | AD-14818 | 667 | CAGGUUCAUGGGUGCCGCATT | 668 | UGCGGCACCCAUGAACCUGTT |
| 615-633 | AD-14819 | 669 | AAAUUGCUGCUGGAGAGGCTT | 670 | GCCUCUCCAGCAGCAAUUUTT |
| 617-635 | AD-14820 | 671 | AUUGCUGCUGGAGAGGCUGTT | 672 | CAGCCUCUCCAGCAGCAAUTT |
| 652-670 | AD-14821 | 673 | UGAAAUUGCAUCCCUUGCUTT | 674 | AGCAAGGGAUGCAAUUUCATT |
| 374-392 | AD-14822 | 675 | UUUUGUUAGAAUUUUUGCUTT | 676 | AGCAAAAAUUCUAACAAAATT |
| 375-393 | AD-14823 | 677 | UUUGUUAGAAUUUUUGCUGTT | 678 | CAGCAAAAAUUCUAACAAATT |
| 631-649 | AD-14824 | 679 | GGCUGCUGCUACUAUAGAATT | 680 | UUCUAUAGUAGCAGCAGCCTT |
| 376-394 | AD-14825 | 681 | UUGUUAGAAUUUUUGCUGGTT | 682 | CCAGCAAAAAUUCUAACAATT |
| 512-530 | AD-14826 | 683 | UUUUUUUGUGUUUUCAGGUTT | 684 | ACCUGAAAACACAAAAAAATT |
| 1127-1145 | AD-14827 | 685 | GAAACUACUUGGGCAAUAGTT | 686 | CUAUUGCCCAAGUAGUUUCTT |
| 1410-1428 | AD-14828 | 687 | AAUGGAUGUUGCCUUUACUTT | 688 | AGUAAAGGCAACAUCCAUUTT |
| 1406-1424 | AD-14829 | 689 | CCUCAAUGGAUGUUGCCUUTT | 690 | AAGGCAACAUCCAUUGAGGTT |
| 1418-1436 | AD-14830 | 691 | UUGCCUUUACUUUUAGGGUTT | 692 | ACCCUAAAAGUAAAGGCAATT |
| 1126-1144 | AD-14831 | 693 | AGAAACUACUUGGGCAAUATT | 694 | UAUUGCCCAAGUAGUUUCUTT |
| 1125-1143 | AD-14832 | 695 | AAGAAACUACUUGGGCAAUTT | 696 | AUUGCCCAAGUAGUUUCUUTT |
| 1419-1437 | AD-14833 | 697 | UGCCUUUACUUUUAGGGUUTT | 698 | AACCCUAAAAGUAAAGGCATT |
| 1420-1438 | AD-14834 | 699 | GCCUUUACUUUUAGGGUUGTT | 700 | CAACCCUAAAAGUAAAGGCTT |
| 1422-1440 | AD-14835 | 701 | CUUUACUUUUAGGGUUGUATT | 702 | UACAACCCUAAAAGUAAAGTT |
| 1423-1441 | AD-14836 | 703 | UUUACUUUUAGGGUUGUACTT | 704 | GUACAACCCUAAAAGUAAATT |
| 1425-1443 | AD-14837 | 705 | UACUUUUAGGGUUGUACGGTT | 706 | CCGUACAACCCUAAAAGUATT |
| 1123-1141 | AD-14838 | 707 | GGAAGAAACUACUUGGGCATT | 708 | UGCCCAAGUAGUUUCUUCCTT |
| 1409-1427 | AD-14839 | 709 | CAAUGGAUGUUGCCUUUACTT | 710 | GUAAAGGCAACAUCCAUUGTT |
| 1413-1431 | AD-14840 | 711 | GGAUGUUGCCUUUACUUUUTT | 712 | AAAAGUAAAGGCAACAUCCTT |
| 1416-1434 | AD-14841 | 713 | UGUUGCCUUUACUUUUAGGTT | 714 | CCUAAAAGUAAAGGCAACATT |
| 1414-1432 | AD-14842 | 715 | GAUGUUGCCUUUACUUUUATT | 716 | UAAAAGUAAAGGCAACAUCTT |
| 911-929 | AD-14843 | 717 | CCAGAAGACUACUAUGAUATT | 718 | UAUCAUAGUAGUCUUCUGGTT |
| 910-928 | AD-14844 | 719 | UCCAGAAGACUACUAUGAUTT | 720 | AUCAUAGUAGUCUUCUGGATT |
| 1120-1138 | AD-14845 | 721 | UUUGGAAGAAACUACUUGGTT | 722 | CCAAGUAGUUUCUUCCAAATT |
| 1404-1422 | AD-14846 | 723 | CUCCUCAAUGGAUGUUGCCTT | 724 | GGCAACAUCCAUUGAGGAGTT |
| 1337-1355 | AD-14847 | 725 | CCAAAUGUGCAAUCUGGUGTT | 726 | CACCAGAUUGCACAUUUGGTT |
| 1338-1356 | AD-14848 | 727 | CAAAUGUGCAAUCUGGUGATT | 728 | UCACCAGAUUGCACAUUUGTT |
| 1397-1415 | AD-14849 | 729 | AGAUCUGCUCCUCAAUGGATT | 730 | UCCAUUGAGGAGCAGAUCUTT |
| 1407-1425 | AD-14850 | 731 | CUCAAUGGAUGUUGCCUUUTT | 732 | AAAGGCAACAUCCAUUGAGTT |
| 4157-4175 | AD-14851 | 733 | GCUCAAAUUUUAUAUAAGATT | 734 | UCUUAUAUAAAAUUUGAGCTT |
| 4795-4813 | AD-14852 | 735 | AGCCUGAUUUUGGUACAUGTT | 736 | CAUGUACCAAAAUCAGGCUTT |
| 4156-4174 | AD-14853 | 737 | CUCAAAUUUUAUAUAAGAATT | 738 | UUCUUAUAUAAAAUUUGAGTT |
| 5002-5020 | AD-14854 | 739 | ACAAAGUGCUGAAUAGGGATT | 740 | UCCCUAUUCAGCACUUUGUTT |
| 4792-4810 | AD-14855 | 741 | CUGAUUUUGGUACAUGGAATT | 742 | UUCCAUGUACCAAAAUCAGTT |
| 4790-4808 | AD-14856 | 743 | GAUUUUGGUACAUGGAAUATT | 744 | UAUUCCAUGUACCAAAAUCTT |
| 4801-4819 | AD-14857 | 745 | CUCAUCAGCCUGAUUUUGGTT | 746 | CCAAAAUCAGGCUGAUGAGTT |
| 4622-4640 | AD-14858 | 747 | AGCCCACUUGUGUGGAUAGTT | 748 | CUAUCCACACAAGUGGGCUTT |
| 4997-5015 | AD-14859 | 749 | GUGCUGAAUAGGGAGGAAUTT | 750 | AUUCCUCCCUAUUCAGCACTT |
| 5094-5112 | AD-14860 | 751 | AGUAAGGGCGUGGAGGCUUTT | 752 | AAGCCUCCACGCCCUUACUTT |
| 4564-4582 | AD-14861 | 753 | GUGACUUAACCCAAGAAGCTT | 754 | GCUUCUUGGGUUAAGUCACTT |
| 5095-5113 | AD-14862 | 755 | UAGUAAGGGCGUGGAGGCUTT | 756 | AGCCUCCACGCCCUUACUATT |
| 4800-4818 | AD-14863 | 757 | UCAUCAGCCUGAUUUUGGUTT | 758 | ACCAAAAUCAGGCUGAUGATT |
| 4265-4283 | AD-14864 | 759 | GUAGAAGACCCUAAAGACUTT | 760 | AGUCUUUAGGGUCUUCUACTT |
| 4267-4285 | AD-14865 | 761 | AGGUAGAAGACCCUAAAGATT | 762 | UCUUUAGGGUCUUCUACCUTT |
| 4270-4288 | AD-14866 | 763 | AAAAGGUAGAAGACCCUAATT | 764 | UUAGGGUCUUCUACCUUUUTT |
| 4269-4287 | AD-14867 | 765 | AAAGGUAGAAGACCCUAAATT | 766 | UUUAGGGUCUUCUACCUUUTT |
| 2874-2892 | AD-14868 | 767 | GAUUGUGCAGUGGAAAGAATT | 768 | UUCUUUCCACUGCACAAUCTT |
| 2875-2893 | AD-14869 | 769 | GGAUUGUGCAGUGGAAAGATT | 770 | UCUUUCCACUGCACAAUCCTT |
| 3950-3968 | AD-14870 | 771 | UGUAGACAGCCAUAUGCAGTT | 772 | CUGCAUAUGGCUGUCUACATT |
| 3896-3914 | AD-14871 | 773 | CAUGACUUUAACCCAGAAGTT | 774 | CUUCUGGGUUAAAGUCAUGTT |
| 4990-5008 | AD-14872 | 775 | AUAGGGAGGAAUCCAUGGATT | 776 | UCCAUGGAUUCCUCCCUAUTT |
| 4994-5012 | AD-14873 | 777 | CUGAAUAGGGAGGAAUCCATT | 778 | UGGAUUCCUCCCUAUUCAGTT |
| 5000-5018 | AD-14874 | 779 | AAAGUGCUGAAUAGGGAGGTT | 780 | CCUCCCUAUUCAGCACUUUTT |
| 4563-4581 | AD-14875 | 781 | UGACUUAACCCAAGAAGCUTT | 782 | AGCUUCUUGGGUUAAGUCATT |
| 3895-3913 | AD-14876 | 783 | AUGACUUUAACCCAGAAGATT | 784 | UCUUCUGGGUUAAAGUCAUTT |
| 4262-4280 | AD-14877 | 785 | GAAGACCCUAAAGACUUUCTT | 786 | GAAAGUCUUUAGGGUCUUCTT |
| 4162-4180 | AD-14878 | 787 | AAAAAGCUCAAAUUUUAUATT | 788 | UAUAAAAUUUGAGCUUUUUTT |
| 4798-4816 | AD-14879 | 789 | AUCAGCCUGAUUUUGGUACTT | 790 | GUACCAAAAUCAGGCUGAUTT |
| 4799-4817 | AD-14880 | 791 | CAUCAGCCUGAUUUUGGUATT | 792 | UACCAAAAUCAGGCUGAUGTT |
| 5006-5024 | AD-14881 | 793 | AUGGACAAAGUGCUGAAUATT | 794 | UAUUCAGCACUUUGUCCAUTT |
| 4264-4282 | AD-14882 | 795 | UAGAAGACCCUAAAGACUUTT | 796 | AAGUCUUUAGGGUCUUCUATT |
| 4268-4286 | AD-14883 | 797 | AAGGUAGAAGACCCUAAAGTT | 798 | CUUUAGGGUCUUCUACCUUTT |
| 4623-4641 | AD-14884 | 799 | CAGCCCACUUGUGUGGAUATT | 800 | UAUCCACACAAGUGGGCUGTT |
| 4788-4806 | AD-14885 | 801 | UUUUGGUACAUGGAAUAGUTT | 802 | ACUAUUCCAUGUACCAAAATT |
| 4993-5011 | AD-14886 | 803 | UGAAUAGGGAGGAAUCCAUTT | 804 | AUGGAUUCCUCCCUAUUCATT |
| 4995-5013 | AD-14887 | 805 | GCUGAAUAGGGAGGAAUCCTT | 806 | GGAUUCCUCCCUAUUCAGCTT |
| 4996-5014 | AD-14888 | 807 | UGCUGAAUAGGGAGGAAUCTT | 808 | GAUUCCUCCCUAUUCAGCATT |
| 3952-3970 | AD-14889 | 809 | UGUGUAGACAGCCAUAUGCTT | 810 | GCAUAUGGCUGUCUACACATT |
| 4595-4613 | AD-14890 | 811 | UGCUUUGAUUGCUUCAGACTT | 812 | GUCUGAAGCAAUCAAAGCATT |
| 4596-4614 | AD-14891 | 813 | UUGCUUUGAUUGCUUCAGATT | 814 | UCUGAAGCAAUCAAAGCAATT |
| 4597-4615 | AD-14892 | 815 | AUUGCUUUGAUUGCUUCAGTT | 816 | CUGAAGCAAUCAAAGCAAUTT |
| 4599-4617 | AD-14893 | 817 | CUAUUGCUUUGAUUGCUUCTT | 818 | GAAGCAAUCAAAGCAAUAGTT |
| 4726-4744 | AD-14894 | 819 | AUUGCAAGGAAUGGCCUAATT | 820 | UUAGGCCAUUCCUUGCAAUTT |
| 4753-4771 | AD-14895 | 821 | AUUUUCCUCCUAAUUCUGATT | 822 | UCAGAAUUAGGAGGAAAAUTT |
| 4802-4820 | AD-14896 | 823 | GCUCAUCAGCCUGAUUUUGTT | 824 | CAAAAUCAGGCUGAUGAGCTT |
| 4803-4821 | AD-14897 | 825 | UGCUCAUCAGCCUGAUUUUTT | 826 | AAAAUCAGGCUGAUGAGCATT |
| 4806-4824 | AD-14898 | 827 | AGUUGCUCAUCAGCCUGAUTT | 828 | AUCAGGCUGAUGAGCAACUTT |
| 5091-5109 | AD-14899 | 829 | AAGGGCGUGGAGGCUUUUUTT | 830 | AAAAAGCCUCCACGCCCUUTT |
| 5093-5111 | AD-14900 | 831 | GUAAGGGCGUGGAGGCUUUTT | 832 | AAAGCCUCCACGCCCUUACTT |
| 4259-4277 | AD-14901 | 833 | GACCCUAAAGACUUUCCUGTT | 834 | CAGGAAAGUCUUUAGGGUCTT |
| 3901-3919 | AD-14902 | 835 | AGGAGCAUGACUUUAACCCTT | 836 | GGGUUAAAGUCAUGCUCCUTT |
| 4757-4775 | AD-14903 | 837 | UGUGAUUUUCCUCCUAAUUTT | 838 | AAUUAGGAGGAAAAUCACATT |
| 4758-4776 | AD-14904 | 839 | UUGUGAUUUUCCUCCUAAUTT | 840 | AUUAGGAGGAAAAUCACAATT |
| 4562-4580 | AD-14905 | 841 | GACUUAACCCAAGAAGCUCTT | 842 | GAGCUUCUUGGGUUAAGUCTT |
| 4585-4603 | AD-14906 | 843 | GCUUCAGACAAUGGUUUGGTT | 844 | CCAAACCAUUGUCUGAAGCTT |
| 4587-4605 | AD-14907 | 845 | UUGCUUCAGACAAUGGUUUTT | 846 | AAACCAUUGUCUGAAGCAATT |
| 4588-4606 | AD-14908 | 847 | AUUGCUUCAGACAAUGGUUTT | 848 | AACCAUUGUCUGAAGCAAUTT |
| 4591-4609 | AD-14909 | 849 | UUGAUUGCUUCAGACAAUGTT | 850 | CAUUGUCUGAAGCAAUCAATT |
| 5003-5021 | AD-14910 | 851 | GACAAAGUGCUGAAUAGGGTT | 852 | CCCUAUUCAGCACUUUGUCTT |
| 4165-4183 | AD-14911 | 853 | AAGAAAAAGCUCAAAUUUUTT | 854 | AAAAUUUGAGCUUUUUCUUTT |
| 4166-4184 | AD-14912 | 855 | AAAGAAAAAGCUCAAAUUUTT | 856 | AAAUUUGAGCUUUUUCUUUTT |
| 4263-4281 | AD-14913 | 857 | AGAAGACCCUAAAGACUUUTT | 858 | AAAGUCUUUAGGGUCUUCUTT |
| 4274-4292 | AD-14914 | 859 | AAAAAAAAGGUAGAAGACCTT | 860 | GGUCUUCUACCUUUUUUUUTT |
| 4266-4284 | AD-14915 | 861 | GGUAGAAGACCCUAAAGACTT | 862 | GUCUUUAGGGUCUUCUACCTT |
| 4272-4290 | AD-14916 | 863 | AAAAAAGGUAGAAGACCCUTT | 864 | AGGGUCUUCUACCUUUUUUTT |
| 4271-4289 | AD-14917 | 865 | AAAAAGGUAGAAGACCCUATT | 866 | UAGGGUCUUCUACCUUUUUTT |
| 4559-4577 | AD-14918 | 867 | UUAACCCAAGAAGCUCUUCTT | 868 | GAAGAGCUUCUUGGGUUAATT |
| 4789-4807 | AD-14919 | 869 | AUUUUGGUACAUGGAAUAGTT | 870 | CUAUUCCAUGUACCAAAAUTT |
| 4998-5016 | AD-14920 | 871 | AGUGCUGAAUAGGGAGGAATT | 872 | UUCCUCCCUAUUCAGCACUTT |
| 5070-5088 | AD-14921 | 873 | GAGGCCAGGGAAAUUCCCUTT | 874 | AGGGAAUUUCCCUGGCCUCTT |
| 4158-4176 | AD-14922 | 875 | AGCUCAAAUUUUAUAUAAGTT | 876 | CUUAUAUAAAAUUUGAGCUTT |
| 5065-5083 | AD-14923 | 877 | CAGGGAAAUUCCCUUGUUUTT | 878 | AAACAAGGGAAUUUCCCUGTT |
| 2872-2890 | AD-14924 | 879 | UUGUGCAGUGGAAAGAAAGTT | 880 | CUUUCUUUCCACUGCACAATT |
| 4782-4800 | AD-14925 | 881 | UACAUGGAAUAGUUCAGAGTT | 882 | CUCUGAACUAUUCCAUGUATT |
| 4783-4801 | AD-14926 | 883 | GUACAUGGAAUAGUUCAGATT | 884 | UCUGAACUAUUCCAUGUACTT |
| 5064-5082 | AD-14927 | 885 | AGGGAAAUUCCCUUGUUUUTT | 886 | AAAACAAGGGAAUUUCCCUTT |
| 5071-5089 | AD-14928 | 887 | GGAGGCCAGGGAAAUUCCCTT | 888 | GGGAAUUUCCCUGGCCUCCTT |
| 3951-3969 | AD-14929 | 889 | GUGUAGACAGCCAUAUGCATT | 890 | UGCAUAUGGCUGUCUACACTT |
| 3949-3967 | AD-14930 | 891 | GUAGACAGCCAUAUGCAGUTT | 892 | ACUGCAUAUGGCUGUCUACTT |
| 4355-4373 | AD-14931 | 893 | GAAGACCUGUUUUGCCAUGTT | 894 | CAUGGCAAAACAGGUCUUCTT |
| 4363-4381 | AD-14932 | 895 | AGUGGGAUGAAGACCUGUUTT | 896 | AACAGGUCUUCAUCCCACUTT |
| 4356-4374 | AD-14933 | 897 | UGAAGACCUGUUUUGCCAUTT | 898 | AUGGCAAAACAGGUCUUCATT |
| 4361-4379 | AD-14934 | 899 | UGGGAUGAAGACCUGUUUUTT | 900 | AAAACAGGUCUUCAUCCCATT |
| 4560-4578 | AD-14935 | 901 | CUUAACCCAAGAAGCUCUUTT | 902 | AAGAGCUUCUUGGGUUAAGTT |
| 2873-2891 | AD-14936 | 903 | AUUGUGCAGUGGAAAGAAATT | 904 | UUUCUUUCCACUGCACAAUTT |
| 4730-4748 | AD-14937 | 905 | CUUUAUUGCAAGGAAUGGCTT | 906 | GCCAUUCCUUGCAAUAAAGTT |
| 3899-3917 | AD-14938 | 907 | GAGCAUGACUUUAACCCAGTT | 908 | CUGGGUUAAAGUCAUGCUCTT |
| 4756-4774 | AD-14939 | 909 | GUGAUUUUCCUCCUAAUUCTT | 910 | GAAUUAGGAGGAAAAUCACTT |
| 4590-4608 | AD-14940 | 911 | UGAUUGCUUCAGACAAUGGTT | 912 | CCAUUGUCUGAAGCAAUCATT |
| 4159-4177 | AD-14941 | 913 | AAGCUCAAAUUUUAUAUAATT | 914 | UUAUAUAAAAUUUGAGCUUTT |
| 2743-2761 | AD-14942 | 915 | CUGGACAUGGAUCAAGCACTT | 916 | GUGCUUGAUCCAUGUCCAGTT |
| 4155-4173 | AD-14943 | 917 | UCAAAUUUUAUAUAAGAAATT | 918 | UUUCUUAUAUAAAAUUUGATT |
| 2871-2889 | AD-14944 | 919 | UGUGCAGUGGAAAGAAAGGTT | 920 | CCUUUCUUUCCACUGCACATT |
| 4786-4804 | AD-14945 | 921 | UUGGUACAUGGAAUAGUUCTT | 922 | GAACUAUUCCAUGUACCAATT |
| 4364-4382 | AD-14946 | 923 | AAGUGGGAUGAAGACCUGUTT | 924 | ACAGGUCUUCAUCCCACUUTT |
| 4359-4377 | AD-14947 | 925 | GGAUGAAGACCUGUUUUGCTT | 926 | GCAAAACAGGUCUUCAUCCTT |
| 2744-2762 | AD-14948 | 927 | UCUGGACAUGGAUCAAGCATT | 928 | UGCUUGAUCCAUGUCCAGATT |
| 4787-4805 | AD-14949 | 929 | UUUGGUACAUGGAAUAGUUTT | 930 | AACUAUUCCAUGUACCAAATT |

TABLE 9
Silencing effect of JCVirus siRNAs

| | Residual | | | Relative | | |
|---|---|---|---|---|---|---|
| | luciferase | | | siRNA | | |
| | activity | | | activity | | |
| | (relative to | SD of | Residual | (normalized | SD of | Relative |
| | control | residual | luciferase | to positive | relative | siRNA |
| Duplex | siRNA treated | luciferase | activity +/− | control luc- | siRNA | activity +/− |
| Name | cells) | activity | SD | siRNA) | activity | SD |
| AD-14742 | 40.85 | 4.38 | 41 ± 4% | 82.24 | 8.83 | 82 ± 9% |
| AD-14743 | 20.92 | 4.1 | 21 ± 4% | 109.97 | 21.56 | 110 ± 22% |
| AD-14744 | 62.2 | 4.47 | 62 ± 4% | 52.56 | 3.78 | 53 ± 4% |
| AD-14745 | 43.97 | 2.6 | 44 ± 3% | 77.91 | 4.6 | 78 ± 5% |
| AD-14746 | 24.52 | 1.96 | 25 ± 2% | 104.96 | 8.38 | 105 ± 8% |
| AD-14747 | 32.67 | 4.51 | 33 ± 5% | 93.62 | 12.94 | 94 ± 13% |
| AD-14748 | 93.99 | 3.23 | 94 ± 3% | 8.36 | 0.29 | 8 ± 0% |
| AD-14749 | 55.16 | 2.81 | 55 ± 3% | 62.35 | 3.18 | 62 ± 3% |
| AD-14750 | 30.86 | 3.11 | 31 ± 3% | 96.14 | 9.7 | 96 ± 10% |
| AD-14751 | 54.44 | 4.03 | 54 ± 4% | 63.35 | 4.69 | 63 ± 5% |
| AD-14752 | 53.88 | 7.58 | 54 ± 8% | 64.13 | 9.02 | 64 ± 9% |
| AD-14753 | 35.24 | 7.45 | 35 ± 7% | 90.05 | 19.03 | 90 ± 19% |
| AD-14754 | 70.39 | 2.8 | 70 ± 3% | 41.17 | 1.64 | 41 ± 2% |
| AD-14755 | 41.8 | 1.6 | 42 ± 2% | 80.93 | 3.1 | 81 ± 3% |
| AD-14756 | 56.69 | 3.05 | 57 ± 3% | 60.22 | 3.24 | 60 ± 3% |
| AD-14757 | 39.16 | 2.16 | 39 ± 2% | 84.6 | 4.67 | 85 ± 5% |
| AD-14758 | 39.79 | 2.95 | 40 ± 3% | 83.72 | 6.22 | 84 ± 6% |
| AD-14759 | 30.62 | 1.01 | 31 ± 1% | 96.48 | 3.2 | 96 ± 3% |
| AD-14760 | 28.14 | 2.74 | 28 ± 3% | 99.93 | 9.72 | 100 ± 10% |
| AD-14761 | 67.42 | 2.83 | 67 ± 3% | 45.3 | 1.9 | 45 ± 2% |
| AD-14762 | 36.1 | 1.3 | 36 ± 1% | 88.85 | 3.21 | 89 ± 3% |
| AD-14763 | 49.39 | 6.77 | 49 ± 7% | 78.14 | 10.71 | 78 ± 11% |
| AD-14764 | 74.04 | 5.32 | 74 ± 5% | 40.09 | 2.88 | 40 ± 3% |
| AD-14765 | 50.84 | 10.47 | 51 ± 10% | 75.91 | 15.63 | 76 ± 16% |
| AD-14766 | 72.59 | 3.55 | 73 ± 4% | 42.32 | 2.07 | 42 ± 2% |
| AD-14767 | 34.82 | 7.41 | 35 ± 7% | 100.63 | 21.4 | 101 ± 21% |
| AD-14768 | 48.68 | 6.31 | 49 ± 6% | 79.24 | 10.27 | 79 ± 10% |
| AD-14769 | 39.07 | 5.53 | 39 ± 6% | 94.08 | 13.31 | 94 ± 13% |
| AD-14770 | 45.59 | 5.89 | 46 ± 6% | 84.01 | 10.85 | 84 ± 11% |
| AD-14771 | 45.57 | 4.1 | 46 ± 4% | 84.04 | 7.56 | 84 ± 8% |
| AD-14772 | 33.12 | 3.64 | 33 ± 4% | 103.26 | 11.36 | 103 ± 11% |
| AD-14773 | 37.38 | 5.72 | 37 ± 6% | 96.69 | 14.78 | 97 ± 15% |
| AD-14774 | 42.38 | 4.41 | 42 ± 4% | 88.96 | 9.26 | 89 ± 9% |
| AD-14775 | 46.59 | 3 | 47 ± 3% | 82.47 | 5.31 | 82 ± 5% |
| AD-14776 | 71.28 | 8.67 | 71 ± 9% | 44.35 | 5.4 | 44 ± 5% |
| AD-14777 | 64.55 | 6.21 | 65 ± 6% | 54.74 | 5.26 | 55 ± 5% |
| AD-14778 | 60.45 | 8.91 | 60 ± 9% | 61.07 | 9 | 61 ± 9% |
| AD-14779 | 32.46 | 0.82 | 32 ± 1% | 104.27 | 2.63 | 104 ± 3% |
| AD-14780 | 22.96 | 2.86 | 23 ± 3% | 118.94 | 14.81 | 119 ± 15% |
| AD-14781 | 56.99 | 9.43 | 57 ± 9% | 66.41 | 10.99 | 66 ± 11% |
| AD-14782 | 29.9 | 8.74 | 30 ± 9% | 108.24 | 31.65 | 108 ± 32% |
| AD-14783 | 42.63 | 6.57 | 43 ± 7% | 88.58 | 13.66 | 89 ± 14% |
| AD-14784 | 67.06 | 1.35 | 67 ± 1% | 50.86 | 1.03 | 51 ± 1% |
| AD-14785 | 48.9 | 3.32 | 49 ± 3% | 78.89 | 5.35 | 79 ± 5% |
| AD-14786 | 27.74 | 2.06 | 28 ± 2% | 111.57 | 8.29 | 112 ± 8% |
| AD-14787 | 38.77 | 6.24 | 39 ± 6% | 94.53 | 15.22 | 95 ± 15% |
| AD-14788 | 32.84 | 8.6 | 33 ± 9% | 103.7 | 27.17 | 104 ± 27% |
| AD-14789 | 46.96 | 1.7 | 47 ± 2% | 81.89 | 2.96 | 82 ± 3% |
| AD-14790 | 43.61 | 4.9 | 44 ± 5% | 87.06 | 9.79 | 87 ± 10% |
| AD-14791 | 35.55 | 4.34 | 36 ± 4% | 99.51 | 12.15 | 100 ± 12% |
| AD-14792 | 38.22 | 3.51 | 38 ± 4% | 95.38 | 8.75 | 95 ± 9% |
| AD-14793 | 90.85 | 5.92 | 91 ± 6% | 14.13 | 0.92 | 14 ± 1% |
| AD-14794 | 83.37 | 3.27 | 83 ± 3% | 25.68 | 1.01 | 26 ± 1% |
| AD-14795 | 55.06 | 3.61 | 55 ± 4% | 69.38 | 4.55 | 69 ± 5% |
| AD-14796 | 30.98 | 5.78 | 31 ± 6% | 106.56 | 19.89 | 107 ± 20% |
| AD-14797 | 28.95 | 3.15 | 29 ± 3% | 109.7 | 11.95 | 110 ± 12% |
| AD-14798 | 67.39 | 3.7 | 67 ± 4% | 50.35 | 2.76 | 50 ± 3% |
| AD-14799 | 66.83 | 4.72 | 67 ± 5% | 51.21 | 3.61 | 51 ± 4% |
| AD-14800 | 33.26 | 5.72 | 33 ± 6% | 103.04 | 17.71 | 103 ± 18% |
| AD-14801 | 39.15 | 4.57 | 39 ± 5% | 93.96 | 10.97 | 94 ± 11% |
| AD-14802 | 91.2 | 5.35 | 91 ± 5% | 13.58 | 0.8 | 14 ± 1% |
| AD-14803 | 34.15 | 7.94 | 34 ± 8% | 101.67 | 23.64 | 102 ± 24% |
| AD-14804 | 30.08 | 6.54 | 30 ± 7% | 107.96 | 23.48 | 108 ± 23% |
| AD-14805 | 32.44 | 4.27 | 32 ± 4% | 104.31 | 13.73 | 104 ± 14% |
| AD-14806 | 35.62 | 3.11 | 36 ± 3% | 99.41 | 8.67 | 99 ± 9% |
| AD-14807 | 28.27 | 7.28 | 28 ± 7% | 110.76 | 28.52 | 111 ± 29% |
| AD-14808 | 30.29 | 3.96 | 30 ± 4% | 107.63 | 14.08 | 108 ± 14% |
| AD-14809 | 31.59 | 4.46 | 32 ± 4% | 105.63 | 14.91 | 106 ± 15% |
| AD-14810 | 30.11 | 5.71 | 30 ± 6% | 107.91 | 20.46 | 108 ± 20% |
| AD-14811 | 55.27 | 6.82 | 55 ± 7% | 69.06 | 8.52 | 69 ± 9% |
| AD-14812 | 45.27 | 5.99 | 45 ± 6% | 84.51 | 11.19 | 85 ± 11% |
| AD-14813 | 77.97 | 7.01 | 78 ± 7% | 34.01 | 3.06 | 34 ± 3% |
| AD-14814 | 29.54 | 3.56 | 30 ± 4% | 108.78 | 13.09 | 109 ± 13% |
| AD-14815 | 65.04 | 3.18 | 65 ± 3% | 53.97 | 2.64 | 54 ± 3% |
| AD-14816 | 64.03 | 4.63 | 64 ± 5% | 55.53 | 4.02 | 56 ± 4% |
| AD-14817 | 37.83 | 2.89 | 38 ± 3% | 95.99 | 7.33 | 96 ± 7% |
| AD-14818 | 28.88 | 5.6 | 29 ± 6% | 109.82 | 21.3 | 110 ± 21% |
| AD-14819 | 92.9 | 4.87 | 93 ± 5% | 10.97 | 0.58 | 11 ± 1% |
| AD-14820 | 75.41 | 3.69 | 75 ± 4% | 37.97 | 1.86 | 38 ± 2% |
| AD-14821 | 73.08 | 6.22 | 73 ± 6% | 41.57 | 3.54 | 42 ± 4% |
| AD-14822 | 86.39 | 9.34 | 86 ± 9% | 21.02 | 2.27 | 21 ± 2% |
| AD-14823 | 96.5 | 10.46 | 97 ± 10% | 5.4 | 0.59 | 5 ± 1% |
| AD-14824 | 32.62 | 3.41 | 33 ± 3% | 104.03 | 10.89 | 104 ± 11% |
| AD-14825 | 102.71 | 7.66 | 103 ± 8% | −4.18 | 0.31 | −4 ± 0% |
| AD-14826 | 92.45 | 5.66 | 92 ± 6% | 11.66 | 0.71 | 12 ± 1% |
| AD-14827 | 63.46 | 16.38 | 63 ± 16% | 46 | 11.88 | 46 ± 12% |
| AD-14828 | 45.99 | 15.21 | 46 ± 15% | 67.99 | 22.49 | 68 ± 22% |
| AD-14829 | 40.54 | 16.03 | 41 ± 16% | 74.86 | 29.6 | 75 ± 30% |
| AD-14830 | 117.1 | 3.66 | 117 ± 4% | −21.52 | 0.67 | −22 ± 1% |
| AD-14831 | 54.78 | 21.12 | 55 ± 21% | 56.93 | 21.95 | 57 ± 22% |
| AD-14832 | 67.07 | 10.81 | 67 ± 11% | 41.46 | 6.68 | 41 ± 7% |
| AD-14833 | 71.52 | 11.9 | 72 ± 12% | 35.85 | 5.97 | 36 ± 6% |
| AD-14834 | 58.05 | 16.37 | 58 ± 16% | 52.81 | 14.89 | 53 ± 15% |
| AD-14835 | 93.36 | 5.43 | 93 ± 5% | 8.36 | 0.49 | 8 ± 0% |
| AD-14836 | 108.84 | 4.85 | 109 ± 5% | −11.13 | 0.5 | −11 ± 0% |
| AD-14837 | 106.68 | 10.06 | 107 ± 10% | −8.41 | 0.79 | −8 ± 1% |
| AD-14838 | 37.06 | 6.68 | 37 ± 7% | 79.23 | 14.28 | 79 ± 14% |
| AD-14839 | 36.03 | 7.54 | 36 ± 8% | 80.53 | 16.84 | 81 ± 17% |
| AD-14840 | 38.51 | 5.9 | 39 ± 6% | 77.4 | 11.86 | 77 ± 12% |
| AD-14841 | 110.86 | 8.91 | 111 ± 9% | −13.67 | 1.1 | −14 ± 1% |
| AD-14842 | 34.83 | 5.51 | 35 ± 6% | 82.04 | 12.98 | 82 ± 13% |
| AD-14843 | 23.75 | 6.04 | 24 ± 6% | 95.99 | 24.41 | 96 ± 24% |
| AD-14844 | 27.47 | 5.29 | 27 ± 5% | 91.3 | 17.57 | 91 ± 18% |
| AD-14845 | 93.12 | 4.7 | 93 ± 5% | 8.67 | 0.44 | 9 ± 0% |
| AD-14846 | 81.72 | 8.26 | 82 ± 8% | 23.01 | 2.33 | 23 ± 2% |
| AD-14847 | 77.89 | 5.29 | 78 ± 5% | 27.83 | 1.89 | 28 ± 2% |
| AD-14848 | 44.4 | 4.95 | 44 ± 5% | 69.99 | 7.81 | 70 ± 8% |
| AD-14849 | 46.41 | 5.08 | 46 ± 5% | 67.46 | 7.38 | 67 ± 7% |
| AD-14850 | 35.52 | 6.7 | 36 ± 7% | 81.17 | 15.31 | 81 ± 15% |
| AD-14851 | 36.07 | 1.13 | 36 ± 1% | 102.63 | 3.22 | 103 ± 3% |
| AD-14852 | 67.98 | 6.75 | 68 ± 7% | 51.41 | 5.11 | 51 ± 5% |
| AD-14853 | 69.44 | 3.07 | 69 ± 3% | 49.05 | 2.17 | 49 ± 2% |
| AD-14854 | 29.12 | 6.88 | 29 ± 7% | 113.79 | 26.89 | 114 ± 27% |
| AD-14855 | 36.04 | 7.07 | 36 ± 7% | 102.68 | 20.14 | 103 ± 20% |
| AD-14856 | 33.61 | 7.93 | 34 ± 8% | 106.57 | 25.15 | 107 ± 25% |
| AD-14857 | 50.76 | 8.76 | 51 ± 9% | 79.04 | 13.64 | 79 ± 14% |
| AD-14858 | 53.6 | 7.26 | 54 ± 7% | 74.49 | 10.09 | 74 ± 10% |
| AD-14859 | 39.07 | 9.34 | 39 ± 9% | 97.82 | 23.38 | 98 ± 23% |
| AD-14860 | 62.78 | 6.85 | 63 ± 7% | 59.75 | 6.52 | 60 ± 7% |
| AD-14861 | 87.47 | 1.86 | 87 ± 2% | 20.12 | 0.43 | 20 ± 0% |
| AD-14862 | 79.95 | 4.02 | 80 ± 4% | 32.19 | 1.62 | 32 ± 2% |
| AD-14863 | 30.46 | 4.49 | 30 ± 4% | 111.64 | 16.46 | 112 ± 16% |
| AD-14864 | 33.18 | 5.07 | 33 ± 5% | 107.26 | 16.38 | 107 ± 16% |
| AD-14865 | 26.25 | 3.98 | 26 ± 4% | 118.39 | 17.96 | 118 ± 18% |
| AD-14866 | 36.73 | 1.24 | 37 ± 1% | 101.57 | 3.44 | 102 ± 3% |
| AD-14867 | 33.16 | 3.13 | 33 ± 3% | 107.3 | 10.12 | 107 ± 10% |
| AD-14868 | 29.91 | 4.56 | 30 ± 5% | 112.52 | 17.16 | 113 ± 17% |
| AD-14869 | 28.24 | 3.66 | 28 ± 4% | 115.2 | 14.91 | 115 ± 15% |
| AD-14870 | 50.37 | 3.04 | 50 ± 3% | 79.67 | 4.81 | 80 ± 5% |
| AD-14871 | 39.37 | 5.11 | 39 ± 5% | 97.32 | 12.63 | 97 ± 13% |
| AD-14872 | 34.71 | 4.12 | 35 ± 4% | 104.82 | 12.43 | 105 ± 12% |
| AD-14873 | 32.14 | 1.79 | 32 ± 2% | 108.93 | 6.07 | 109 ± 6% |
| AD-14874 | 101.77 | 4.87 | 102 ± 5% | −2.85 | 0.14 | −3 ± 0% |
| AD-14875 | 80.81 | 4.39 | 81 ± 4% | 30.8 | 1.67 | 31 ± 2% |
| AD-14876 | 30.74 | 1.88 | 31 ± 2% | 111.18 | 6.81 | 111 ± 7% |
| AD-14877 | 57.38 | 2.84 | 57 ± 3% | 68.42 | 3.39 | 68 ± 3% |
| AD-14878 | 70.23 | 3.35 | 70 ± 3% | 47.79 | 2.28 | 48 ± 2% |
| AD-14879 | 79.03 | 7.72 | 79 ± 8% | 33.66 | 3.29 | 34 ± 3% |
| AD-14880 | 21.65 | 2.46 | 22 ± 2% | 125.78 | 14.28 | 126 ± 14% |
| AD-14881 | 27.66 | 1.71 | 28 ± 2% | 116.13 | 7.17 | 116 ± 7% |
| AD-14882 | 34.01 | 2.94 | 34 ± 3% | 105.93 | 9.16 | 106 ± 9% |
| AD-14883 | 40.62 | 3.22 | 41 ± 3% | 95.33 | 7.56 | 95 ± 8% |
| AD-14884 | 35.73 | 5.94 | 36 ± 6% | 103.18 | 17.14 | 103 ± 17% |
| AD-14885 | 47.4 | 7.65 | 47 ± 8% | 84.45 | 13.63 | 84 ± 14% |
| AD-14886 | 37.23 | 3.94 | 37 ± 4% | 100.76 | 10.67 | 101 ± 11% |
| AD-14887 | 42.94 | 7.26 | 43 ± 7% | 91.61 | 15.5 | 92 ± 15% |
| AD-14888 | 32.58 | 4.06 | 33 ± 4% | 108.24 | 13.5 | 108 ± 14% |
| AD-14889 | 83.09 | 2.98 | 83 ± 3% | 27.15 | 0.97 | 27 ± 1% |
| AD-14890 | 59.49 | 2.94 | 59 ± 3% | 65.04 | 3.22 | 65 ± 3% |
| AD-14891 | 21.93 | 5.52 | 22 ± 6% | 125.32 | 31.52 | 125 ± 32% |
| AD-14892 | 72.69 | 2.19 | 73 ± 2% | 43.84 | 1.32 | 44 ± 1% |
| AD-14893 | 24.43 | 7.07 | 24 ± 7% | 121.32 | 35.11 | 121 ± 35% |
| AD-14894 | 33.84 | 5.08 | 34 ± 5% | 106.2 | 15.95 | 106 ± 16% |
| AD-14895 | 21.68 | 4.46 | 22 ± 4% | 125.73 | 25.84 | 126 ± 26% |
| AD-14896 | 26.99 | 5.01 | 27 ± 5% | 117.2 | 21.73 | 117 ± 22% |
| AD-14897 | 29.04 | 2.72 | 29 ± 3% | 113.92 | 10.67 | 114 ± 11% |
| AD-14898 | 32.64 | 4.87 | 33 ± 5% | 108.14 | 16.13 | 108 ± 16 |
| AD-14899 | 61.71 | 4.59 | 62 ± 5% | 61.47 | 4.57 | 61 ± 5% |
| AD-14900 | 31.01 | 2.84 | 31 ± 3% | 110.75 | 10.14 | 111 ± 10% |
| AD-14901 | 31.47 | 1.57 | 31 ± 2% | 110.01 | 5.49 | 110 ± 5% |
| AD-14902 | 76.99 | 0.55 | 77 ± 1% | 36.95 | 0.26 | 37 ± 0% |
| AD-14903 | 20.55 | 3.55 | 21 ± 4% | 127.55 | 22.05 | 128 ± 22% |
| AD-14904 | 22.65 | 6.87 | 23 ± 7% | 124.18 | 37.68 | 124 ± 38% |
| AD-14905 | 56.98 | 4.94 | 57 ± 5% | 69.07 | 5.99 | 69 ± 6% |
| AD-14906 | 34.2 | 3.66 | 34 ± 4% | 105.63 | 11.29 | 106 ± 11% |
| AD-14907 | 28.59 | 8.12 | 29 ± 8% | 114.64 | 32.56 | 115 ± 33% |
| AD-14908 | 34.08 | 3.36 | 34 ± 3% | 105.82 | 10.44 | 106 ± 10% |
| AD-14909 | 76.57 | 2.33 | 77 ± 2% | 37.61 | 1.15 | 38 ± 1% |
| AD-14910 | 46.5 | 4.14 | 46 ± 4% | 85.89 | 7.64 | 86 ± 8% |
| AD-14911 | 29.62 | 2.02 | 30 ± 2% | 112.99 | 7.69 | 113 ± 8% |
| AD-14912 | 22.27 | 0.48 | 22 ± 0% | 124.78 | 2.69 | 125 ± 3% |
| AD-14913 | 59.8 | 2.85 | 60 ± 3% | 64.53 | 3.08 | 65 ± 3% |
| AD-14914 | 93.21 | 5.1 | 93 ± 5% | 10.9 | 0.6 | 11 ± 1% |
| AD-14915 | 25.99 | 4.45 | 26 ± 4% | 118.82 | 20.34 | 119 ± 20% |
| AD-14916 | 48.2 | 1.46 | 48 ± 1% | 83.16 | 2.51 | 83 ± 3% |
| AD-14917 | 41.03 | 3.07 | 41 ± 3% | 94.67 | 7.08 | 95 ± 7% |
| AD-14918 | 110.62 | 6.34 | 111 ± 6% | −17.04 | 0.98 | −17 ± 1% |
| AD-14919 | 73.66 | 3.68 | 74 ± 4% | 42.29 | 2.11 | 42 ± 2% |
| AD-14920 | 19.8 | 1.72 | 20 ± 2% | 128.75 | 11.2 | 129 ± 11% |
| AD-14921 | 33.13 | 1.14 | 33 ± 1% | 107.34 | 3.71 | 107 ± 4% |
| AD-14922 | 52.94 | 6.99 | 53 ± 7% | 63.41 | 8.37 | 63 ± 8% |
| AD-14923 | 33.77 | 8.92 | 34 ± 9% | 89.23 | 23.56 | 89 ± 24% |
| AD-14924 | 64.47 | 10.96 | 64 ± 11% | 47.86 | 8.13 | 48 ± 8% |
| AD-14925 | 97.16 | 7.57 | 97 ± 8% | 3.83 | 0.3 | 4 ± 0% |
| AD-14926 | 27.29 | 8.79 | 27 ± 9% | 97.96 | 31.56 | 98 ± 32% |
| AD-14927 | 27.02 | 10.01 | 27 ± 10% | 98.33 | 36.42 | 98 ± 36% |
| AD-14928 | 76.75 | 4.78 | 77 ± 5% | 31.32 | 1.95 | 31 ± 2% |
| AD-14929 | 32.92 | 9.44 | 33 ± 9% | 90.38 | 25.93 | 90 ± 26% |
| AD-14930 | 31 | 9.4 | 31 ± 9% | 92.97 | 28.21 | 93 ± 28% |
| AD-14931 | 31.36 | 8.73 | 31 ± 9% | 92.48 | 25.74 | 92 ± 26% |
| AD-14932 | 32.42 | 9.01 | 32 ± 9% | 91.05 | 25.29 | 91 ± 25% |
| AD-14933 | 39.94 | 6.96 | 40 ± 7% | 80.92 | 14.1 | 81 ± 14% |
| AD-14934 | 42.94 | 7.66 | 43 ± 8% | 76.88 | 13.71 | 77 ± 14% |
| AD-14935 | 47.74 | 8.48 | 48 ± 8% | 70.41 | 12.51 | 70 ± 13% |
| AD-14936 | 35.21 | 4.02 | 35 ± 4% | 87.29 | 9.97 | 87 ± 10% |
| AD-14937 | 89.25 | 3.53 | 89 ± 4% | 14.48 | 0.57 | 14 ± 1% |
| AD-14938 | 29.38 | 6.46 | 29 ± 6% | 95.15 | 20.91 | 95 ± 21% |
| AD-14939 | 26.45 | 8.33 | 26 ± 8% | 99.09 | 31.21 | 99 ± 31% |
| AD-14940 | 77.5 | 6.51 | 78 ± 7% | 30.31 | 2.55 | 30 ± 3% |
| AD-14941 | 36.4 | 10.76 | 36 ± 11% | 85.68 | 25.32 | 86 ± 25% |
| AD-14942 | 65.11 | 5.84 | 65 ± 6% | 47.01 | 4.22 | 47 ± 4% |
| AD-14943 | 89.96 | 4.69 | 90 ± 5% | 13.52 | 0.71 | 14 ± 1% |
| AD-14944 | 48.98 | 5.85 | 49 ± 6% | 68.74 | 8.21 | 69 ± 8% |
| AD-14945 | 43.45 | 3.29 | 43 ± 3% | 76.18 | 5.77 | 76 ± 6% |
| AD-14946 | 41.25 | 1.26 | 41 ± 1% | 79.15 | 2.43 | 79 ± 2% |
| AD-14947 | 42.1 | 7.34 | 42 ± 7% | 78.01 | 13.6 | 78 ± 14% |
| AD-14948 | 42.39 | 5.75 | 42 ± 6% | 77.62 | 10.54 | 78 ± 11% |
| AD-14949 | 27.68 | 5.79 | 28 ± 6% | 97.44 | 20.39 | 97 ± 20% |

TABLE 10
siRNAs targeting JCV transcripts for primary screen.

| | | | SEQ | | | |
|---|---|---|---|---|---|---|
| Duplex | | Position in | ID | Sequence | SEQ | Sequence |
| Name | Chemistry | Consensus | NO: | (5′--> 3′) | ID NO: | (5′--> 3′) |

| | | | | | | |
|---|---|---|---|---|---|---|
| AD-12598 | a | 1426-1444 | 1 | AcuuuuAGGGuuGuAcGGGTsT | 2 | CcCGuAcAACCCuAAAAGUTsT |
| AD-12708 | b | 1426-1444 | 3 | AcuuuuAGGGuuGuAcGGGTsT | 4 | CCCGuAcAACCCuAAAAGUTsT |
| AD-12599 | a | 1427-1445 | 5 | cuuuuAGGGuuGuAcGGGATsT | 6 | UcCCGuAcAACCCuAAAAGTsT |
| AD-12709 | b | 1427-1445 | 7 | cuuuuAGGGuuGuAcGGGATsT | 8 | UCCCGuAcAACCCuAAAAGTsT |
| AD-12600 | a | 2026-2044 | 9 | cAGAGcAcAAGGcGuAccuTsT | 10 | AgGuACGCCUUGUGCUCUGTsT |
| AD-12710 | b | 2026-2044 | 11 | cAGAGcAcAAGGcGuAccuTsT | 12 | AGGuACGCCUUGUGCUCUGTsT |
| AD-12784 | c | 2026-2044 | 13 | caGAGcAcAAGGcGuAccuTsT | 14 | AGGuACGCCUUGUGCUCUGTsT |
| AD-12832 | d | 2026-2044 | 15 | caGAGcAcAAGGcGuAccuTsT | 16 | AgGuACGCCUUGUGCUCUGTsT |
| AD-12601 | a | 1431-1449 | 17 | uAGGGuuGuAcGGGAcuGuTsT | 18 | AcAGUCCCGuAcAACCCuATsT |
| AD-12785 | c | 1431-1449 | 19 | uaGGGuuGuAcGGGAcuGuTsT | 20 | AcAGUCCCGuAcAACCCuATsT |
| AD-12602 | a | 1432-1450 | 21 | AGGGuuGuAcGGGAcuGuATsT | 22 | uacAGUCCCGuAcAACCCUTsT |
| AD-12711 | b | 1432-1450 | 23 | AGGGuuGuAcGGGAcuGuATsT | 24 | uAcAGUCCCGuAcAACCCUTsT |
| AD-12786 | c | 1432-1450 | 25 | AgGGuuGuAcGGGAcuGuATsT | 26 | uAcAGUCCCGuAcAACCCUTsT |
| AD-12833 | d | 1432-1450 | 27 | AgGGuuGuAcGGGAcuGuATsT | 28 | uacAGUCCCGuAcAACCCUTsT |
| AD-12603 | a | 1436-1454 | 29 | uuGuAcGGGAcuGuAAcAcTsT | 30 | GuGUuAcAGUCCCGuAcAATsT |
| AD-12712 | b | 1436-1454 | 31 | uuGuAcGGGAcuGuAAcAcTsT | 32 | GUGUuAcAGUCCCGuAcAATsT |
| AD-12604 | a | 4794-4812 | 33 | GccuGAuuuuGGuAcAuGGTsT | 34 | CcAUGuACcAAAAUcAGGCTsT |
| AD-12605 | a | 5099-5117 | 35 | GAAGuAGuAAGGGcGuGGATsT | 36 | UccACGCCCUuACuACUUCTsT |
| AD-12713 | b | 5099-5117 | 37 | GAAGuAGuAAGGGcGuGGATsT | 38 | UCcACGCCCUuACuACUUCTsT |
| AD-12787 | c | 5099-5117 | 39 | GaAGuAGuAAGGGcGuGGATsT | 40 | UCcACGCCCUuACuACUUCTsT |
| AD-12834 | d | 5099-5117 | 41 | GaAGuAGuAAGGGcGuGGATsT | 42 | UccACGCCCUuACuACUUCTsT |
| AD-12606 | a | 713-731 | 43 | AuAGGccuuAcuccuGAAATsT | 44 | UuUcAGGAGuAAGGCCuAUTsT |
| AD-12714 | b | 713-731 | 45 | AuAGGccuuAcuccuGAAATsT | 46 | UUUcAGGAGuAAGGCCuAUTsT |
| AD-12607 | a | 3946-3964 | 47 | GAcAGccAuAuGcAGuAGuTsT | 48 | AcuACUGcAuAUGGCUGUCTsT |
| AD-12715 | b | 3946-3964 | 49 | GAcAGccAuAuGcAGuAGuTsT | 50 | ACuACUGcAuAUGGCUGUCTsT |
| AD-12788 | c | 3946-3964 | 51 | GacAGccAuAuGcAGuAGuTsT | 52 | ACuACUGcAuAUGGCUGUCTsT |
| AD-12835 | d | 3946-3964 | 53 | GacAGccAuAuGcAGuAGuTsT | 54 | AcuACUGcAuAUGGCUGUCTsT |
| AD-12608 | a | 1128-1146 | 55 | AAAcuAcuuGGGcAAuAGuTsT | 56 | AcuAUUGCCcAAGuAGUUUTsT |
| AD-12716 | b | 1128-1146 | 57 | AAAcuAcuuGGGcAAuAGuTsT | 58 | ACuAUUGCCcAAGuAGUUUTsT |
| AD-12789 | c | 1128-1146 | 59 | AaAcuAcuuGGGcAAuAGuTsT | 60 | ACuAUUGCCcAAGuAGUUUTsT |
| AD-12836 | d | 1128-1146 | 61 | AaAcuAcuuGGGcAAuAGuTsT | 62 | AcuAUUGCCcAAGuAGUUUTsT |
| AD-12609 | a | 525-543 | 63 | ucAGGuucAuGGGuGccGcTsT | 64 | GcGGcACCcAUGAACCUGATsT |
| AD-12717 | b | 525-543 | 65 | ucAGGuucAuGGGuGccGcTsT | 66 | GCGGcACCcAUGAACCUGATsT |
| AD-12610 | a | 5096-5114 | 67 | GuAGuAAGGGcGuGGAGGcTsT | 68 | GcCUCcACGCCCUuACuACTsT |
| AD-12718 | b | 5096-5114 | 69 | GuAGuAAGGGcGuGGAGGcTsT | 70 | GCCUCcACGCCCUuACuACTsT |
| AD-12611 | a | 4727-4745 | 71 | uAuuGcAAGGAAuGGccuATsT | 72 | uaGGCcAUUCCUUGcAAuATsT |
| AD-12719 | b | 4727-4745 | 73 | uAuuGcAAGGAAuGGccuATsT | 74 | uAGGCcAUUCCUUGcAAuATsT |
| AD-12790 | c | 4727-4745 | 75 | uauuGcAAGGAAuGGccuATsT | 76 | uAGGCcAUUCCUUGcAAuATsT |
| AD-12837 | d | 4727-4745 | 77 | uauuGcAAGGAAuGGccuATsT | 78 | uaGGCcAUUCCUUGcAAuATsT |
| AD-12612 | a | 5097-5115 | 79 | AGuAGuAAGGGcGuGGAGGTsT | 80 | CcUCcACGCCCUuACuACUTsT |
| AD-12720 | b | 5097-5115 | 81 | AGuAGuAAGGGcGuGGAGGTsT | 82 | CCUCcACGCCCUuACuACUTsT |
| AD-12791 | c | 5097-5115 | 83 | AguAGuAAGGGcGuGGAGGTsT | 84 | CCUCcACGCCCUuACuACUTsT |
| AD-12838 | d | 5097-5115 | 85 | AguAGuAAGGGcGuGGAGGTsT | 86 | CcUCcACGCCCUuACuACUTsT |
| AD-12613 | a | 4601-4619 | 87 | uGcuAuuGcuuuGAuuGcuTsT | 88 | AgcAAUcAAAGcAAuAGcATsT |
| AD-12721 | b | 4601-4619 | 89 | uGcuAuuGcuuuGAuuGcuTsT | 90 | AGcAAUcAAAGcAAuAGcATsT |
| AD-12792 | c | 4601-4619 | 91 | ugcuAuuGcuuuGAuuGcuTsT | 92 | AGcAAUcAAAGcAAuAGcATsT |
| AD-12839 | d | 4601-4619 | 93 | ugcuAuuGcuuuGAuuGcuTsT | 94 | AgcAAUcAAAGcAAuAGcATsT |
| AD-12614 | a | 4600-4618 | 95 | GcuAuuGcuuuGAuuGcuuTsT | 96 | AaGcAAUcAAAGcAAuAGCTsT |
| AD-12722 | b | 4600-4618 | 97 | GcuAuuGcuuuGAuuGcuuTsT | 98 | AAGcAAUcAAAGcAAuAGCTsT |
| AD-12615 | a | 1421-1439 | 99 | ccuuuAcuuuuAGGGuuGuTsT | 100 | AcAACCCuAAAAGuAAAGGTsT |
| AD-12616 | a | 1424-1442 | 101 | uuAcuuuuAGGGuuGuAcGTsT | 102 | CguAcAACCCuAAAAGuAATsT |
| AD-12723 | b | 1424-1442 | 103 | uuAcuuuuAGGGuuGuAcGTsT | 104 | CGuAcAACCCuAAAAGuAATsT |
| AD-12617 | a | 1403-1421 | 105 | GcuccucAAuGGAuGuuGcTsT | 106 | GcAAcAUCcAUUGAGGAGCTsT |
| AD-12618 | a | 1534-1552 | 107 | uuAuAAGAGGAGGAGuAGATsT | 108 | UcuACUCCUCCUCUuAuAATsT |
| AD-12724 | b | 1534-1552 | 109 | uuAuAAGAGGAGGAGuAGATsT | 110 | UCuACUCCUCCUCUuAuAATsT |
| AD-12619 | a | 5098-5116 | 111 | AAGuAGuAAGGGcGuGGAGTsT | 112 | CuCcACGCCCUuACuACUUTsT |
| AD-12725 | b | 5098-5116 | 113 | AAGuAGuAAGGGcGuGGAGTsT | 114 | CUCcACGCCCUuACuACUUTsT |
| AD-12793 | c | 5098-5116 | 115 | AaGuAGuAAGGGcGuGGAGTsT | 116 | CUCcACGCCCUuACuACUUTsT |
| AD-12840 | d | 5098-5116 | 117 | AaGuAGuAAGGGcGuGGAGTsT | 118 | CuCcACGCCCUuACuACUUTsT |
| AD-12620 | a | 1430-1448 | 119 | uuAGGGuuGuAcGGGAcuGTsT | 120 | caGUCCCGuAcAACCCuAATsT |
| AD-12726 | b | 1430-1448 | 121 | uuAGGGuuGuAcGGGAcuGTsT | 122 | cAGUCCCGuAcAACCCuAATsT |
| AD-12621 | a | 1701-1719 | 123 | GAcAuGcuuccuuGuuAcATsT | 124 | UguAAcAAGGAAGcAUGUCTsT |
| AD-12727 | b | 1701-1719 | 125 | GAcAuGcuuccuuGuuAcATsT | 126 | UGuAAcAAGGAAGcAUGUCTsT |
| AD-12794 | c | 1701-1719 | 127 | GacAuGcuuccuuGuuAcATsT | 128 | UGuAAcAAGGAAGcAUGUCTsT |
| AD-12841 | d | 1701-1719 | 129 | GacAuGcuuccuuGuuAcATsT | 130 | UguAAcAAGGAAGcAUGUCTsT |
| AD-12622 | a | 2066-2084 | 131 | uGuuGAAuGuuGGGuuccuTsT | 132 | AgGAACCcAAcAUUcAAcATsT |
| AD-12728 | b | 2066-2084 | 133 | uGuuGAAuGuuGGGuuccuTsT | 134 | AGGAACCcAAcAUUcAAcATsT |
| AD-12795 | c | 2066-2084 | 135 | uguuGAAuGuuGGGuuccuTsT | 136 | AGGAACCcAAcAUUcAAcATsT |
| AD-12842 | d | 2066-2084 | 137 | uguuGAAuGuuGGGuuccuTsT | 138 | AgGAACCcAAcAUUcAAcATsT |
| AD-12623 | a | 4561-4579 | 139 | AcuuAAcccAAGAAGcucuTsT | 140 | AgAGCUUCUUGGGUuAAGUTsT |
| AD-12729 | b | 4561-4579 | 141 | AcuuAAcccAAGAAGcucuTsT | 142 | AGAGCUUCUUGGGUuAAGUTsT |
| AD-12624 | a | 4797-4815 | 143 | ucAGccuGAuuuuGGuAcATsT | 144 | UguACcAAAAUcAGGCUGATsT |
| AD-12730 | b | 4797-4815 | 145 | ucAGccuGAuuuuGGuAcATsT | 146 | UGuACcAAAAUcAGGCUGATsT |
| AD-12625 | a | 1428-1446 | 147 | uuuuAGGGuuGuAcGGGAcTsT | 148 | GuCCCGuAcAACCCuAAAATsT |
| AD-12731 | b | 1428-1446 | 149 | uuuuAGGGuuGuAcGGGAcTsT | 150 | GUCCCGuAcAACCCuAAAATsT |
| AD-12626 | a | 1429-1447 | 151 | uuuAGGGuuGuAcGGGAcuTsT | 152 | AgUCCCGuAcAACCCuAAATsT |
| AD-12732 | b | 1429-1447 | 153 | uuuAGGGuuGuAcGGGAcuTsT | 154 | AGUCCCGuAcAACCCuAAATsT |
| AD-12627 | a | 662-680 | 155 | ucccuuGcuAcuGuAGAGGTsT | 156 | CcUCuAcAGuAGcAAGGGATsT |
| AD-12733 | b | 662-680 | 157 | ucccuuGcuAcuGuAGAGGTsT | 158 | CCUCuAcAGuAGcAAGGGATsT |
| AD-12628 | a | 663-681 | 59 | cccuuGcuAcuGuAGAGGGTsT | 160 | CcCUCuAcAGuAGcAAGGGTsT |
| AD-12734 | b | 663-681 | 161 | cccuuGcuAcuGuAGAGGGTsT | 162 | CCCUCuAcAGuAGcAAGGGTsT |
| AD-12629 | a | 1402-1420 | 163 | uGcuccucAAuGGAuGuuGTsT | 164 | caAcAUCcAUUGAGGAGcATsT |
| AD-12735 | b | 1402-1420 | 165 | uGcuccucAAuGGAuGuuGTsT | 166 | cAAcAUCcAUUGAGGAGcATsT |
| AD-12796 | c | 1402-1420 | 167 | ugcuccucAAuGGAuGuuGTsT | 168 | cAAcAUCcAUUGAGGAGcATsT |
| AD-12843 | d | 1402-1420 | 169 | ugcuccucAAuGGAuGuuGTsT | 170 | caAcAUCcAUUGAGGAGcATsT |
| AD-12630 | a | 1398-1416 | 171 | GAucuGcuccucAAuGGAuTsT | 172 | AuCcAUUGAGGAGcAGAUCTsT |
| AD-12736 | b | 1398-1416 | 173 | GAucuGcuccucAAuGGAuTsT | 174 | AUCcAUUGAGGAGcAGAUCTsT |
| AD-12797 | c | 1398-1416 | 175 | GaucuGcuccucAAuGGAuTsT | 176 | AUCcAUUGAGGAGcAGAUCTsT |
| AD-12844 | d | 1398-1416 | 177 | GaucuGcuccucAAuGGAuTsT | 178 | AuCcAUUGAGGAGcAGAUCTsT |
| AD-12631 | a | 1399-1417 | 179 | AucuGcuccucAAuGGAuGTsT | 180 | caUCcAUUGAGGAGcAGAUTsT |
| AD-12737 | b | 1399-1417 | 181 | AucuGcuccucAAuGGAuGTsT | 182 | cAUCcAUUGAGGAGcAGAUTsT |
| AD-12632 | a | 1400-1418 | 183 | ucuGcuccucAAuGGAuGuTsT | 184 | AcAUCcAUUGAGGAGcAGATsT |
| AD-12633 | a | 1401-1419 | 185 | cuGcuccucAAuGGAuGuuTsT | 186 | AacAUCcAUUGAGGAGcAGTsT |
| AD-12738 | b | 1401-1419 | 187 | cuGcuccucAAuGGAuGuuTsT | 188 | AAcAUCcAUUGAGGAGcAGTsT |
| AD-12634 | a | 1435-1453 | 189 | GuuGuAcGGGAcuGuAAcATsT | 190 | UgUuAcAGUCCCGuAcAACTsT |
| AD-12739 | b | 1435-1453 | 191 | GuuGuAcGGGAcuGuAAcATsT | 192 | UGUuAcAGUCCCGuAcAACTsT |
| AD-12635 | a | 1437-1455 | 193 | uGuAcGGGAcuGuAAcAccTsT | 194 | GgUGUuAcAGUCCCGuAcATsT |
| AD-12740 | b | 1437-1455 | 195 | uGuAcGGGAcuGuAAcAccTsT | 196 | GGUGUuAcAGUCCCGuAcATsT |
| AD-12798 | c | 1437-1455 | 197 | uguAcGGGAcuGuAAcAccTsT | 198 | GGUGUuAcAGUCCCGuAcATsT |
| AD-12845 | d | 1437-1455 | 199 | uguAcGGGAcuGuAAcAccTsT | 200 | GgUGUuAcAGUCCCGuAcATsT |
| AD-12636 | a | 1438-1456 | 201 | GuAcGGGAcuGuAAcAccuTsT | 202 | AgGUGUuAcAGUCCCGuACTsT |
| AD-12741 | b | 1438-1456 | 203 | GuAcGGGAcuGuAAcAccuTsT | 204 | AGGUGUuAcAGUCCCGuACTsT |
| AD-12637 | a | 4796-4814 | 205 | cAGccuGAuuuuGGuAcAuTsT | 206 | AuGuACcAAAAUcAGGCUGTsT |
| AD-12742 | b | 4796-4814 | 207 | cAGccuGAuuuuGGuAcAuTsT | 208 | AUGuACcAAAAUcAGGCUGTsT |
| AD-12799 | c | 4796-4814 | 209 | caGccuGAuuuuGGuAcAuTsT | 210 | AUGuACcAAAAUcAGGCUGTsT |
| AD-12846 | d | 4796-4814 | 211 | caGccuGAuuuuGGuAcAuTsT | 212 | AuGuACcAAAAUcAGGCUGTsT |
| AD-12638 | a | 4992-5010 | 213 | GAAuAGGGAGGAAuccAuGTsT | 214 | caUGGAUUCCUCCCuAUUCTsT |
| AD-12743 | b | 4992-5010 | 215 | GAAuAGGGAGGAAuccAuGTsT | 216 | cAUGGAUUCCUCCCuAUUCTsT |
| AD-12800 | c | 4992-5010 | 217 | GaAuAGGGAGGAAuccAuGTsT | 218 | cAUGGAUUCCUCCCuAUUCTsT |
| AD-12847 | d | 4992-5010 | 219 | GaAuAGGGAGGAAuccAuGTsT | 220 | caUGGAUUCCUCCCuAUUCTsT |
| AD-12639 | a | 4999-5017 | 221 | AAGuGcuGAAuAGGGAGGATsT | 222 | UcCUCCCuAUUcAGcACUUTsT |
| AD-12744 | b | 4999-5017 | 223 | AAGuGcuGAAuAGGGAGGATsT | 224 | UCCUCCCuAUUcAGcACUUTsT |
| AD-12801 | c | 4999-5017 | 225 | AaGuGcuGAAuAGGGAGGATsT | 226 | UCCUCCCuAUUcAGcACUUTsT |
| AD-12848 | d | 4999-5017 | 227 | AaGuGcuGAAuAGGGAGGATsT | 228 | UcCUCCCuAUUcAGcACUUTsT |
| AD-12640 | a | 630-648 | 229 | AGGcuGcuGcuAcuAuAGATsT | 230 | UcuAuAGuAGcAGcAGCCUTsT |
| AD-12745 | b | 630-648 | 231 | AGGcuGcuGcuAcuAuAGATsT | 232 | UCuAuAGuAGcAGcAGCCUTsT |
| AD-12802 | c | 630-648 | 233 | AgGcuGcuGcuAcuAuAGATsT | 234 | UCuAuAGuAGcAGcAGCCUTsT |
| AD-12849 | d | 630-648 | 235 | AgGcuGcuGcuAcuAuAGATsT | 236 | UcuAuAGuAGcAGcAGCCUTsT |
| AD-12641 | a | 3947-3965 | 237 | AGAcAGccAuAuGcAGuAGTsT | 238 | CuACUGcAuAUGGCUGUCUTsT |
| AD-12803 | c | 3947-3965 | 239 | AgAcAGccAuAuGcAGuAGTsT | 240 | CuACUGcAuAUGGCUGUCUTsT |
| AD-12642 | a | 524-542 | 241 | uucAGGuucAuGGGuGccGTsT | 242 | CgGcACCcAUGAACCUGAATsT |
| AD-12746 | b | 524-542 | 243 | uucAGGuucAuGGGuGccGTsT | 244 | CGGcACCcAUGAACCUGAATsT |
| AD-12643 | a | 3948-3966 | 245 | uAGAcAGccAuAuGcAGuATsT | 246 | uaCUGcAuAUGGCUGUCuATsT |
| AD-12747 | b | 3948-3966 | 247 | uAGAcAGccAuAuGcAGuATsT | 248 | uACUGcAuAUGGCUGUCuATsT |
| AD-12804 | c | 3948-3966 | 249 | uaGAcAGccAuAuGcAGuATsT | 250 | uACUGcAuAUGGCUGUCuATsT |
| AD-12850 | d | 3948-3966 | 251 | uaGAcAGccAuAuGcAGuATsT | 252 | uaCUGcAuAUGGCUGUCuATsT |
| AD-12644 | a | 3900-3918 | 253 | GGAGcAuGAcuuuAAcccATsT | 254 | UgGGUuAAAGUcAUGCUCCTsT |
| AD-12748 | b | 3900-3918 | 255 | GGAGcAuGAcuuuAAcccATsT | 256 | UGGGUuAAAGUcAUGCUCCTsT |
| AD-12805 | c | 3900-3918 | 257 | GgAGcAuGAcuuuAAcccATsT | 258 | UGGGUuAAAGUcAUGCUCCTsT |
| AD-12851 | d | 3900-3918 | 259 | GgAGcAuGAcuuuAAcccATsT | 260 | UgGGUuAAAGUcAUGCUCCTsT |
| AD-12645 | a | 1417-1435 | 261 | GuuGccuuuAcuuuuAGGGTsT | 262 | CcCuAAAAGuAAAGGcAACTsT |
| AD-12749 | b | 1417-1435 | 263 | GuuGccuuuAcuuuuAGGGTsT | 264 | CCCuAAAAGuAAAGGcAACTsT |
| AD-12646 | a | 4565-4583 | 265 | uGuGAcuuAAcccAAGAAGTsT | 266 | CuUCUUGGGUuAAGUcAcATsT |
| AD-12750 | b | 4565-4583 | 267 | uGuGAcuuAAcccAAGAAGTsT | 268 | CUUCUUGGGUuAAGUcAcATsT |
| AD-12806 | c | 4565-4583 | 269 | uguGAcuuAAcccAAGAAGTsT | 270 | CUUCUUGGGUuAAGUcAcATsT |
| AD-12852 | d | 4565-4583 | 271 | uguGAcuuAAcccAAGAAGTsT | 272 | CuUCUUGGGUuAAGUcAcATsT |
| AD-12647 | a | 4598-4616 | 273 | uAuuGcuuuGAuuGcuucATsT | 274 | UgAAGcAAUcAAAGcAAuATsT |
| AD-12751 | b | 4598-4616 | 275 | uAuuGcuuuGAuuGcuucATsT | 276 | UGAAGcAAUcAAAGcAAuATsT |
| AD-12807 | c | 4598-4616 | 277 | uauuGcuuuGAuuGcuucATsT | 278 | UGAAGcAAUcAAAGcAAuATsT |
| AD-12853 | d | 4598-4616 | 279 | uauuGcuuuGAuuGcuucATsT | 280 | UgAAGcAAUcAAAGcAAuATsT |
| AD-12648 | a | 2060-2078 | 281 | AuAuccuGuuGAAuGuuGGTsT | 282 | CcAAcAUUcAAcAGGAuAUTsT |
| AD-12649 | a | 4729-4747 | 283 | uuuAuuGcAAGGAAuGGccTsT | 284 | GgCcAUUCCUUGcAAuAAATsT |
| AD-12752 | b | 4729-4747 | 285 | uuuAuuGcAAGGAAuGGccTsT | 286 | GGCcAUUCCUUGcAAuAAATsT |
| AD-12650 | a | 1122-1140 | 287 | uGGAAGAAAcuAcuuGGGcTsT | 288 | GcCcAAGuAGUUUCUUCcATsT |
| AD-12753 | b | 1122-1140 | 289 | uGGAAGAAAcuAcuuGGGcTsT | 290 | GCCcAAGuAGUUUCUUCcATsT |
| AD-12808 | c | 1122-1140 | 291 | ugGAAGAAAcuAcuuGGGcTsT | 292 | GCCcAAGuAGUUUCUUCcATsT |
| AD-12854 | d | 1122-1140 | 293 | ugGAAGAAAcuAcuuGGGcTsT | 294 | GcCcAAGuAGUUUCUUCcATsT |
| AD-12651 | a | 4261-4279 | 295 | AAGAcccuAAAGAcuuuccTsT | 296 | GgAAAGUCUUuAGGGUCUUTsT |
| AD-12754 | b | 4261-4279 | 297 | AAGAcccuAAAGAcuuuccTsT | 298 | GGAAAGUCUUuAGGGUCUUTsT |
| AD-12809 | c | 4261-4279 | 299 | AaGAcccuAAAGAcuuuccTsT | 300 | GGAAAGUCUUuAGGGUCUUTsT |
| AD-12855 | d | 4261-4279 | 301 | AaGAcccuAAAGAcuuuccTsT | 302 | GgAAAGUCUUuAGGGUCUUTsT |
| AD-12652 | a | 1412-1430 | 303 | uGGAuGuuGccuuuAcuuuTsT | 304 | AaAGuAAAGGcAAcAUCcATsT |
| AD-12755 | b | 1412-1430 | 305 | uGGAuGuuGccuuuAcuuuTsT | 306 | AAAGuAAAGGcAAcAUCcATsT |
| AD-12810 | c | 1412-1430 | 307 | ugGAuGuuGccuuuAcuuuTsT | 308 | AAAGuAAAGGcAAcAUCcATsT |
| AD-12856 | d | 1412-1430 | 309 | ugGAuGuuGccuuuAcuuuTsT | 310 | AaAGuAAAGGcAAcAUCcATsT |
| AD-12653 | a | 4592-4610 | 311 | uuuGAuuGcuucAGAcAAuTsT | 312 | AuUGUCUGAAGcAAUcAAATsT |
| AD-12756 | b | 4592-4610 | 313 | uuuGAuuGcuucAGAcAAuTsT | 314 | AUUGUCUGAAGcAAUcAAATsT |
| AD-12654 | a | 4991-5009 | 315 | AAuAGGGAGGAAuccAuGGTsT | 316 | CcAUGGAUUCCUCCCuAUUTsT |
| AD-12811 | c | 4991-5009 | 317 | AauAGGGAGGAAuccAuGGTsT | 318 | CcAUGGAUUCCUCCCuAUUTsT |
| AD-12655 | a | 5004-5022 | 319 | GGAcAAAGuGcuGAAuAGGTsT | 320 | CcuAUUcAGcACUUUGUCCTsT |
| AD-12757 | b | 5004-5022 | 321 | GGAcAAAGuGcuGAAuAGGTsT | 322 | CCuAUUcAGcACUUUGUCCTsT |
| AD-12812 | c | 5004-5022 | 323 | GgAcAAAGuGcuGAAuAGGTsT | 324 | CCuAUUcAGcACUUUGUCCTsT |
| AD-12857 | d | 5004-5022 | 325 | GgAcAAAGuGcuGAAuAGGTsT | 326 | CcuAUUcAGcACUUUGUCCTsT |
| AD-12656 | a | 5005-5023 | 327 | uGGAcAAAGuGcuGAAuAGTsT | 328 | CuAUUcAGcACUUUGUCcATsT |
| AD-12813 | c | 5005-5023 | 329 | ugGAcAAAGuGcuGAAuAGTsT | 330 | CuAUUcAGcACUUUGUCcATsT |
| AD-12657 | a | 654-672 | 331 | AAAuuGcAucccuuGcuAcTsT | 332 | GuAGcAAGGGAUGcAAUUUTsT |
| AD-12814 | c | 654-672 | 333 | AaAuuGcAucccuuGcuAcTsT | 334 | GuAGcAAGGGAUGcAAUUUTsT |
| AD-12658 | a | 659-677 | 335 | GcAucccuuGcuAcuGuAGTsT | 336 | CuAcAGuAGcAAGGGAUGCTsT |
| AD-12659 | a | 4273-4291 | 337 | AAAAAAAGGuAGAAGAcccTsT | 338 | GgGUCUUCuACCUUUUUUUTsT |
| AD-12758 | b | 4273-4291 | 339 | AAAAAAAGGuAGAAGAcccTsT | [symbol] | [symbol] |
| AD-12815 | c | 4273-4291 | 341 | AaAAAAAGGuAGAAGAcccTsT | 342 | GGGUCUUCuACCUUUUUUUTsT |
| AD-12858 | d | 4273-4291 | 343 | AaAAAAAGGuAGAAGAcccTsT | 344 | GgGUCUUCuACCUUUUUUUTsT |
| AD-12660 | a | 2025-2043 | 345 | AcAGAGcAcAAGGcGuAccTsT | 346 | GguACGCCUUGUGCUCUGUTsT |
| AD-12759 | b | 2025-2043 | 347 | AcAGAGcAcAAGGcGuAccTsT | 348 | GGuACGCCUUGUGCUCUGUTsT |
| AD-12661 | a | 4791-4809 | 349 | uGAuuuuGGuAcAuGGAAuTsT | 350 | AuUCcAUGuACcAAAAUcATsT |
| AD-12760 | b | 4791-4809 | 351 | uGAuuuuGGuAcAuGGAAuTsT | 352 | AUUCcAUGuACcAAAAUcATsT |
| AD-12816 | c | 4791-4809 | 353 | ugAuuuuGGuAcAuGGAAuTsT | 354 | AUUCcAUGuACcAAAAUcATsT |
| AD-12859 | d | 4791-4809 | 355 | ugAuuuuGGuAcAuGGAAuTsT | 356 | AuUCcAUGuACcAAAAUcATsT |
| AD-12662 | a | 1433-1451 | 357 | GGGuuGuAcGGGAcuGuAATsT | 358 | UuAcAGUCCCGuAcAACCCTsT |
| AD-12817 | c | 1433-1451 | 359 | GgGuuGuAcGGGAcuGuAATsT | 360 | UuAcAGUCCCGuAcAACCCTsT |
| AD-12663 | a | 1434-1452 | 361 | GGuuGuAcGGGAcuGuAAcTsT | 362 | GuuAcAGUCCCGuAcAACCTsT |
| AD-12761 | b | 1434-1452 | 363 | GGuuGuAcGGGAcuGuAAcTsT | 364 | GUuAcAGUCCCGuAcAACCTsT |
| AD-12818 | c | 1434-1452 | 365 | GguuGuAcGGGAcuGuAAcTsT | 366 | GUuAcAGUCCCGuAcAACCTsT |
| AD-12860 | d | 1434-1452 | 367 | GguuGuAcGGGAcuGuAAcTsT | 368 | GuuAcAGUCCCGuAcAACCTsT |
| AD-12664 | a | 1440-1458 | 369 | AcGGGAcuGuAAcAccuGcTsT | 370 | GcAGGUGUuAcAGUCCCGUTsT |
| AD-12665 | a | 1442-1460 | 371 | GGGAcuGuAAcAccuGcucTsT | 372 | GaGcAGGUGUuAcAGUCCCTsT |
| AD-12762 | b | 1442-1460 | 373 | GGGAcuGuAAcAccuGcucTsT | 374 | GAGcAGGUGUuAcAGUCCCTsT |
| AD-12819 | c | 1442-1460 | 375 | GgGAcuGuAAcAccuGcucTsT | 376 | GAGcAGGUGUuAcAGUCCCTsT |
| AD-12861 | d | 1442-1460 | 377 | GgGAcuGuAAcAccuGcucTsT | 378 | GaGcAGGUGUuAcAGUCCCTsT |
| AD-12666 | a | 1608-1626 | 379 | AcuccAGAAAuGGGuGAccTsT | 380 | GgUcACCcAUUUCUGGAGUTsT |
| AD-12763 | b | 1608-1626 | 381 | AcuccAGAAAuGGGuGAccTsT | 382 | GGUcACCcAUUUCUGGAGUTsT |
| AD-12667 | a | 4793-4811 | 383 | ccuGAuuuuGGuAcAuGGATsT | 384 | UccAUGuACcAAAAUcAGGTsT |
| AD-12764 | b | 4793-4811 | 385 | ccuGAuuuuGGuAcAuGGATsT | 386 | UCcAUGuACcAAAAUcAGGTsT |
| AD-12668 | a | 5001-5019 | 387 | cAAAGuGcuGAAuAGGGAGTsT | 388 | CuCCCuAUUcAGcACUUUGTsT |
| AD-12765 | b | 5001-5019 | 389 | cAAAGuGcuGAAuAGGGAGTsT | 390 | CUCCCuAUUcAGcACUUUGTsT |
| AD-12820 | c | 5001-5019 | 391 | caAAGuGcuGAAuAGGGAGTsT | 392 | CUCCCuAUUcAGcACUUUGTsT |
| AD-12862 | d | 5001-5019 | 393 | caAAGuGcuGAAuAGGGAGTsT | 394 | CuCCCuAUUcAGcACUUUGTsT |
| AD-12669 | a | 5066-5084 | 395 | ccAGGGAAAuucccuuGuuTsT | 396 | AacAAGGGAAUUUCCCUGGTsT |
| AD-12766 | b | 5066-5084 | 397 | ccAGGGAAAuucccuuGuuTsT | 398 | AAcAAGGGAAUUUCCCUGGTsT |
| AD-12670 | a | 5069-5087 | 399 | AGGccAGGGAAAuucccuuTsT | 400 | AaGGGAAUUUCCCUGGCCUTsT |
| AD-12767 | b | 5069-5087 | 401 | AGGccAGGGAAAuucccuuTsT | 402 | AAGGGAAUUUCCCUGGCCUTsT |
| AD-12821 | c | 5069-5087 | 403 | AgGccAGGGAAAuucccuuTsT | 404 | AAGGGAAUUUCCCUGGCCUTsT |
| AD-12863 | d | 5069-5087 | 405 | AgGccAGGGAAAuucccuuTsT | 406 | AaGGGAAUUUCCCUGGCCUTsT |
| AD-12671 | a | 564-582 | 407 | uAGuuGcuAcuGuuucuGATsT | 408 | UcAGAAAcAGuAGcAACuATsT |
| AD-12822 | c | 564-582 | 409 | uaGuuGcuAcuGuuucuGATsT | 410 | UcAGAAAcAGuAGcAACuATsT |
| AD-12672 | a | 633-651 | 411 | cuGcuGcuAcuAuAGAAGuTsT | 412 | AcUUCuAuAGuAGcAGcAGTsT |
| AD-12768 | b | 633-651 | 413 | cuGcuGcuAcuAuAGAAGuTsT | 414 | ACUUCuAuAGuAGcAGcAGTsT |
| AD-12673 | a | 634-652 | 415 | uGcuGcuAcuAuAGAAGuuTsT | 416 | AaCUUCuAuAGuAGcAGcATsT |
| AD-12769 | b | 634-652 | 417 | uGcuGcuAcuAuAGAAGuuTsT | 418 | AACUUCuAuAGuAGcAGcATsT |
| AD-12823 | c | 634-652 | 419 | ugcuGcuAcuAuAGAAGuuTsT | 420 | AACUUCuAuAGuAGcAGcATsT |
| AD-12864 | d | 634-652 | 421 | ugcuGcuAcuAuAGAAGuuTsT | 422 | AaCUUCuAuAGuAGcAGcATsT |
| AD-12674 | a | 635-653 | 423 | GcuGcuAcuAuAGAAGuuGTsT | 424 | caACUUCuAuAGuAGcAGCTsT |
| AD-12770 | b | 635-653 | 425 | GcuGcuAcuAuAGAAGuuGTsT | 426 | cAACUUCuAuAGuAGcAGCTsT |
| AD-12675 | a | 636-654 | 427 | cuGcuAcuAuAGAAGuuGATsT | 428 | UcAACUUCuAuAGuAGcAGTsT |
| AD-12676 | a | 637-655 | 429 | uGcuAcuAuAGAAGuuGAATsT | 430 | UucAACUUCuAuAGuAGcATsT |
| AD-12771 | b | 637-655 | 431 | uGcuAcuAuAGAAGuuGAATsT | 432 | UUcAACUUCuAuAGuAGcATsT |
| AD-12824 | c | 637-655 | 433 | ugcuAcuAuAGAAGuuGAATsT | 434 | UUcAACUUCuAuAGuAGcATsT |
| AD-12865 | d | 637-655 | 435 | ugcuAcuAuAGAAGuuGAATsT | 436 | UucAACUUCuAuAGuAGcATsT |
| AD-12677 | a | 912-930 | 437 | cAGAAGAcuAcuAuGAuAuTsT | 438 | AuAUcAuAGuAGUCUUCUGTsT |
| AD-12825 | c | 912-930 | 439 | caGAAGAcuAcuAuGAuAuTsT | 440 | AuAUcAuAGuAGUCUUCUGTsT |
| AD-12678 | a | 4153-4171 | 441 | AAAuuuuAuAuAAGAAAcuTsT | 442 | AgUUUCUuAuAuAAAAUUUTsT |
| AD-12772 | b | 4153-4171 | 443 | AAAuuuuAuAuAAGAAAcuTsT | 444 | AGUUUCUuAuAuAAAAUUUTsT |
| AD-12826 | c | 4153-4171 | 445 | AaAuuuuAuAuAAGAAAcuTsT | 446 | AGUUUCUuAuAuAAAAUUUTsT |
| AD-12866 | d | 4153-4171 | 447 | AaAuuuuAuAuAAGAAAcuTsT | 448 | AgUUUCUuAuAuAAAAUUUTsT |
| AD-12679 | a | 4779-4797 | 449 | AuGGAAuAGuucAGAGGuuTsT | 450 | AaCCUCUGAACuAUUCcAUTsT |
| AD-12773 | b | 4779-4797 | 451 | AuGGAAuAGuucAGAGGuuTsT | 452 | AACCUCUGAACuAUUCcAUTsT |
| AD-12680 | a | 4780-4798 | 453 | cAuGGAAuAGuucAGAGGuTsT | 454 | AcCUCUGAACuAUUCcAUGTsT |
| AD-12774 | b | 4780-4798 | 455 | cAuGGAAuAGuucAGAGGuTsT | 456 | ACCUCUGAACuAUUCcAUGTsT |
| AD-12827 | c | 4780-4798 | 457 | cauGGAAuAGuucAGAGGuTsT | 458 | ACCUCUGAACuAUUCcAUGTsT |
| AD-12867 | d | 4780-4798 | 459 | cauGGAAuAGuucAGAGGuTsT | 460 | AcCUCUGAACuAUUCcAUGTsT |
| AD-12681 | a | 4781-4799 | 461 | AcAuGGAAuAGuucAGAGGTsT | 462 | CcUCUGAACuAUUCcAUGUTsT |
| AD-12775 | b | 4781-4799 | 463 | AcAuGGAAuAGuucAGAGGTsT | 464 | CCUCUGAACuAUUCcAUGUTsT |
| AD-12682 | a | 4784-4802 | 465 | GGuAcAuGGAAuAGuucAGTsT | 466 | CuGAACuAUUCcAUGuACCTsT |
| AD-12776 | b | 4784-4802 | 467 | GGuAcAuGGAAuAGuucAGTsT | 468 | CUGAACuAUUCcAUGuACCTsT |
| AD-12828 | c | 4784-4802 | 469 | GguAcAuGGAAuAGuucAGTsT | 470 | CUGAACuAUUCcAUGuACCTsT |
| AD-12868 | d | 4784-4802 | 471 | GguAcAuGGAAuAGuucAGTsT | 472 | CuGAACuAUUCcAUGuACCTsT |
| AD-12683 | a | 4785-4803 | 473 | uGGuAcAuGGAAuAGuucATsT | 474 | UgAACuAUUCcAUGuACcATsT |
| AD-12777 | b | 4785-4803 | 475 | uGGuAcAuGGAAuAGuucATsT | 476 | UGAACuAUUCcAUGuACcATsT |
| AD-12829 | c | 4785-4803 | 477 | ugGuAcAuGGAAuAGuucATsT | 478 | UGAACuAUUCcAUGuACcATsT |
| AD-12869 | d | 4785-4803 | 479 | ugGuAcAuGGAAuAGuucATsT | 480 | UgAACuAUUCcAUGuACcATsT |
| AD-12684 | a | 719-737 | 481 | cuuAcuccuGAAAcAuAuGTsT | 482 | cauAUGUUUcAGGAGuAAGTsT |
| AD-12778 | b | 719-737 | 483 | cuuAcuccuGAAAcAuAuGTsT | 484 | cAuAUGUUUcAGGAGuAAGTsT |
| AD-12685 | a | 909-927 | 485 | AuccAGAAGAcuAcuAuGATsT | 486 | UcAuAGuAGUCUUCUGGAUTsT |
| AD-12686 | a | 1119-1137 | 487 | uuuuGGAAGAAAcuAcuuGTsT | 488 | caAGuAGUUUCUUCcAAAATsT |
| AD-12779 | b | 1119-1137 | 489 | uuuuGGAAGAAAcuAcuuGTsT | 490 | cAAGuAGUUUCUUCcAAAATsT |
| AD-12687 | a | 1121-1139 | 491 | uuGGAAGAAAcuAcuuGGGTsT | 492 | CccAAGuAGUUUCUUCcAATsT |
| AD-12780 | b | 1121-1139 | 493 | uuGGAAGAAAcuAcuuGGGTsT | 494 | CCcAAGuAGUUUCUUCcAATsT |
| AD-12688 | a | 4357-4375 | 495 | AuGAAGAccuGuuuuGccATsT | 496 | UgGcAAAAcAGGUCUUcAUTsT |
| AD-12781 | b | 4357-4375 | 497 | AuGAAGAccuGuuuuGccATsT | 498 | UGGcAAAAcAGGUCUUcAUTsT |
| AD-12689 | a | 4358-4376 | 499 | GAuGAAGAccuGuuuuGccTsT | 500 | GgcAAAAcAGGUCUUcAUCTsT |
| AD-12782 | b | 4358-4376 | 501 | GAuGAAGAccuGuuuuGccTsT | 502 | GGcAAAAcAGGUCUUcAUCTsT |
| AD-12830 | c | 4358-4376 | 503 | GauGAAGAccuGuuuuGccTsT | 504 | GGcAAAAcAGGUCUUcAUCTsT |
| AD-12870 | d | 4358-4376 | 505 | GauGAAGAccuGuuuuGccTsT | 506 | GgcAAAAcAGGUCUUcAUCTsT |
| AD-12690 | a | 4360-4378 | 507 | GGGAuGAAGAccuGuuuuGTsT | 508 | caAAAcAGGUCUUcAUCCCTsT |
| AD-12783 | b | 4360-4378 | 509 | GGGAuGAAGAccuGuuuuGTsT | 510 | cAAAAcAGGUCUUcAUCCCTsT |
| AD-12831 | c | 4360-4378 | 511 | GgGAuGAAGAccuGuuuuGTsT | 512 | cAAAAcAGGUCUUcAUCCCTsT |
| AD-12871 | d | 4360-4378 | 513 | GgGAuGAAGAccuGuuuuGTsT | 514 | caAAAcAGGUCUUcAUCCCTsT |

[symbol]

indicates data missing or illegible when filed

TABLE 11
Description of chemistries in Table 10

| Chemistry | Description |
|---|---|
| a | exo/endo-light + 2′-O-methyl in position 2 of |
| | antisense |
| b | exo/endo-light: sense strand: dTsdT + 2′OMe@all |
| | Py; antisense strand: dTsdT + |
| | 2′OMe@ Py in uA, cA |
| c | exo/endo-light + 2′-O-methyl in position 2 of sense |
| d | exo/endo-light + 2′-O-methyl in position 2 of sense |
| | and antisense |

TABLE 12
Silencing effect of modified JC Virus dsRNAs

| | | | | Relative | | |
|---|---|---|---|---|---|---|
| | Residual | | | siRNA | | |
| | luciferase | | | activity | | |
| | activity | SD of | Residual | (normalized | SD of | |
| | (relative to | residual | luciferase | to positive | relative | |
| | control siRNA | luciferace | activity +/− | control luc- | siRNA | Relative siRNA |
| Duplex Name | treated cells) | activity | SD | siRNA) | activity | activity +/− SD |
| AD-12598 | 91 | 11 | 91 ± 11% | 9 | 2 | 9 ± 2% |
| AD-12708 | 32 | 5 | 32 ± 5% | 76 | 17 | 76 ± 17% |
| AD-12599 | 25 | 6 | 25 ± 6% | 79 | 13 | 79 ± 13% |
| AD-12709 | 16 | 4 | 16 ± 4% | 97 | 26 | 97 ± 26% |
| AD-12600 | 79 | 9 | 79 ± 9% | 21 | 3 | 21 ± 3% |
| AD-12710 | 25 | 4 | 25 ± 4% | 85 | 24 | 85 ± 24% |
| AD-12784 | 23 | 2 | 23 ± 2% | 87 | 14 | 87 ± 14% |
| AD-12832 | 84 | 11 | 84 ± 11% | 18 | 4 | 18 ± 4% |
| AD-12601 | 102 | 8 | 102 ± 8% | −6 | 1 | −6 ± 1% |
| AD-12785 | 95 | 10 | 95 ± 10% | 6 | 1 | 6 ± 1% |
| AD-12602 | 107 | 9 | 107 ± 9% | −11 | 2 | −11 ± 2% |
| AD-12711 | 70 | 4 | 70 ± 4% | 34 | 3 | 34 ± 3% |
| AD-12786 | 69 | 8 | 69 ± 8% | 35 | 7 | 35 ± 7% |
| AD-12833 | 94 | 8 | 94 ± 8% | 7 | 1 | 7 ± 1% |
| AD-12603 | 100 | 9 | 100 ± 9% | −4 | 1 | −4 ± 1% |
| AD-12712 | 27 | 5 | 27 ± 5% | 82 | 16 | 82 ± 16% |
| AD-12604 | 15 | 2 | 15 ± 2% | 94 | 13 | 94 ± 13% |
| AD-12605 | 94 | 5 | 94 ± 5% | 7 | 0 | 7 ± 0% |
| AD-12713 | 61 | 10 | 61 ± 10% | 41 | 8 | 41 ± 8% |
| AD-12787 | 55 | 6 | 55 ± 6% | 47 | 6 | 47 ± 6% |
| AD-12834 | 92 | 16 | 92 ± 16% | 8 | 2 | 8 ± 2% |
| AD-12606 | 78 | 3 | 78 ± 3% | 25 | 1 | 25 ± 1% |
| AD-12714 | 63 | 6 | 63 ± 6% | 42 | 5 | 42 ± 5% |
| AD-12607 | 101 | 9 | 101 ± 9% | −1 | 0 | −1 ± 0% |
| AD-12715 | 101 | 5 | 101 ± 5% | −1 | 0 | −1 ± 0% |
| AD-12788 | 85 | 18 | 85 ± 18% | 15 | 4 | 15 ± 4% |
| AD-12835 | 95 | 9 | 95 ± 9% | 6 | 1 | 6 ± 1% |
| AD-12608 | 103 | 13 | 103 ± 13% | −3 | 0 | −3 ± 0% |
| AD-12716 | 81 | 9 | 81 ± 9% | 22 | 3 | 22 ± 3% |
| AD-12789 | 61 | 4 | 61 ± 4% | 44 | 4 | 44 ± 4% |
| AD-12836 | 103 | 11 | 103 ± 11% | −3 | 0 | −3 ± 0% |
| AD-12609 | 108 | 19 | 108 ± 19% | −9 | 2 | −9 ± 2% |
| AD-12717 | 94 | 17 | 94 ± 17% | 7 | 1 | 7 ± 1% |
| AD-12610 | 88 | 9 | 88 ± 9% | 14 | 2 | 14 ± 2% |
| AD-12718 | 39 | 4 | 39 ± 4% | 64 | 8 | 64 ± 8% |
| AD-12611 | 38 | 6 | 38 ± 6% | 69 | 12 | 69 ± 12% |
| AD-12719 | 26 | 4 | 26 ± 4% | 78 | 13 | 78 ± 13% |
| AD-12790 | 17 | 3 | 17 ± 3% | 87 | 18 | 87 ± 18% |
| AD-12837 | 22 | 4 | 22 ± 4% | 81 | 16 | 81 ± 16% |
| AD-12612 | 100 | 6 | 100 ± 6% | 0 | 0 | 0 ± 0% |
| AD-12720 | 73 | 6 | 73 ± 6% | 28 | 3 | 28 ± 3% |
| AD-12791 | 46 | 9 | 46 ± 9% | 57 | 12 | 57 ± 12% |
| AD-12838 | 97 | 15 | 97 ± 15% | 3 | 1 | 3 ± 1% |
| AD-12613 | 26 | 4 | 26 ± 4% | 82 | 15 | 82 ± 15% |
| AD-12721 | 10 | 1 | 10 ± 1% | 94 | 12 | 94 ± 12% |
| AD-12792 | 10 | 3 | 10 ± 3% | 94 | 40 | 94 ± 40% |
| AD-12839 | 22 | 3 | 22 ± 3% | 81 | 12 | 81 ± 12% |
| AD-12614 | 15 | 5 | 15 ± 5% | 94 | 38 | 94 ± 38% |
| AD-12722 | 6 | 1 | 6 ± 1% | 98 | 26 | 98 ± 26% |
| AD-12615 | 93 | 4 | 93 ± 4% | 8 | 0 | 8 ± 0% |
| AD-12616 | 95 | 4 | 95 ± 4% | 5 | 0 | 5 ± 0% |
| AD-12723 | 73 | 7 | 73 ± 7% | 30 | 3 | 30 ± 3% |
| AD-12617 | 88 | 10 | 88 ± 10% | 13 | 2 | 13 ± 2% |
| AD-12618 | 42 | 7 | 42 ± 7% | 60 | 7 | 60 ± 7% |
| AD-12724 | 21 | 5 | 21 ± 5% | 89 | 32 | 89 ± 32% |
| AD-12619 | 95 | 7 | 95 ± 7% | 6 | 1 | 6 ± 1% |
| AD-12725 | 71 | 2 | 71 ± 2% | 30 | 1 | 30 ± 1% |
| AD-12793 | 54 | 7 | 54 ± 7% | 48 | 7 | 48 ± 7% |
| AD-12840 | 94 | 9 | 94 ± 9% | 7 | 1 | 7 ± 1% |
| AD-12620 | 106 | 7 | 106 ± 7% | −8 | 1 | −8 ± 1% |
| AD-12726 | 100 | 7 | 100 ± 7% | 0 | 0 | 0 ± 0% |
| AD-12621 | 107 | 9 | 107 ± 9% | −7 | 1 | −7 ± 1% |
| AD-12727 | 47 | 4 | 47 ± 4% | 60 | 8 | 60 ± 8% |
| AD-12794 | 40 | 8 | 40 ± 8% | 67 | 20 | 67 ± 20% |
| AD-12841 | 78 | 13 | 78 ± 13% | 25 | 8 | 25 ± 8% |
| AD-12622 | 16 | 4 | 16 ± 4% | 92 | 29 | 92 ± 29% |
| AD-12728 | 25 | 6 | 25 ± 6% | 84 | 29 | 84 ± 29% |
| AD-12795 | 23 | 3 | 23 ± 3% | 86 | 20 | 86 ± 20% |
| AD-12842 | 19 | 4 | 19 ± 4% | 91 | 20 | 91 ± 20% |
| AD-12623 | 103 | 9 | 103 ± 9% | −3 | 0 | −3 ± 0% |
| AD-12729 | 84 | 8 | 84 ± 8% | 17 | 2 | 17 ± 2% |
| AD-12624 | 31 | 4 | 31 ± 4% | 77 | 12 | 77 ± 12% |
| AD-12730 | 18 | 1 | 18 ± 1% | 85 | 3 | 85 ± 3% |
| AD-12625 | 94 | 10 | 94 ± 10% | 5 | 1 | 5 ± 1% |
| AD-12731 | 57 | 4 | 57 ± 4% | 48 | 4 | 48 ± 4% |
| AD-12626 | 99 | 7 | 99 ± 7% | 0 | 0 | 0 ± 0% |
| AD-12732 | 82 | 5 | 82 ± 5% | 20 | 1 | 20 ± 1% |
| AD-12627 | 80 | 6 | 80 ± 6% | 22 | 2 | 22 ± 2% |
| AD-12733 | 65 | 6 | 65 ± 6% | 39 | 4 | 39 ± 4% |
| AD-12628 | 81 | 6 | 81 ± 6% | 21 | 2 | 21 ± 2% |
| AD-12734 | 82 | 7 | 82 ± 7% | 21 | 2 | 21 ± 2% |
| AD-12629 | 113 | 11 | 113 ± 11% | −14 | 2 | −14 ± 2% |
| AD-12735 | 90 | 9 | 90 ± 9% | 11 | 1 | 11 ± 1% |
| AD-12796 | 92 | 8 | 92 ± 8% | 9 | 1 | 9 ± 1% |
| AD-12843 | 117 | 7 | 117 ± 7% | −19 | 1 | −19 ± 1% |
| AD-12630 | 124 | 3 | 124 ± 3% | −27 | 1 | −27 ± 1% |
| AD-12736 | 85 | 4 | 85 ± 4% | 16 | 1 | 16 ± 1% |
| AD-12797 | 52 | 1 | 52 ± 1% | 53 | 1 | 53 ± 1% |
| AD-12844 | 96 | 4 | 96 ± 4% | 5 | 0 | 5 ± 0% |
| AD-12631 | 110 | 11 | 110 ± 11% | −12 | 1 | −12 ± 1% |
| AD-12737 | 115 | 13 | 115 ± 13% | −17 | 2 | −17 ± 2% |
| AD-12632 | 106 | 2 | 106 ± 2% | −7 | 0 | −7 ± 0% |
| AD-12633 | 107 | 12 | 107 ± 12% | −8 | 1 | −8 ± 1% |
| AD-12738 | 88 | 5 | 88 ± 5% | 14 | 1 | 14 ± 1% |
| AD-12634 | 79 | 5 | 79 ± 5% | 24 | 1 | 24 ± 1% |
| AD-12739 | 69 | 8 | 69 ± 8% | 35 | 6 | 35 ± 6% |
| AD-12635 | 75 | 8 | 75 ± 8% | 25 | 6 | 25 ± 6% |
| AD-12740 | 65 | 8 | 65 ± 8% | 40 | 8 | 40 ± 8% |
| AD-12798 | 56 | 4 | 56 ± 4% | 50 | 6 | 50 ± 6% |
| AD-12845 | 74 | 6 | 74 ± 6% | 30 | 3 | 30 ± 3% |
| AD-12636 | 89 | 8 | 89 ± 8% | 9 | 1 | 9 ± 1% |
| AD-12741 | 31 | 4 | 31 ± 4% | 78 | 14 | 78 ± 14% |
| AD-12637 | 16 | 2 | 16 ± 2% | 93 | 14 | 93 ± 14% |
| AD-12742 | 18 | 3 | 18 ± 3% | 85 | 14 | 85 ± 14% |
| AD-12799 | 18 | 4 | 18 ± 4% | 86 | 22 | 86 ± 22% |
| AD-12846 | 15 | 2 | 15 ± 2% | 89 | 14 | 89 ± 14% |
| AD-12638 | 95 | 6 | 95 ± 6% | 5 | 0 | 5 ± 0% |
| AD-12743 | 23 | 4 | 23 ± 4% | 81 | 15 | 81 ± 15% |
| AD-12800 | 14 | 1 | 14 ± 1% | 90 | 10 | 90 ± 10% |
| AD-12847 | 90 | 12 | 90 ± 12% | 10 | 2 | 10 ± 2% |
| AD-12639 | 113 | 11 | 113 ± 11% | −15 | 2 | −15 ± 2% |
| AD-12744 | 42 | 4 | 42 ± 4% | 60 | 7 | 60 ± 7% |
| AD-12801 | 34 | 3 | 34 ± 3% | 68 | 8 | 68 ± 8% |
| AD-12848 | 114 | 3 | 114 ± 3% | −14 | 0 | −14 ± 0% |
| AD-12640 | 96 | 11 | 96 ± 11% | 4 | 1 | 4 ± 1% |
| AD-12745 | 52 | 7 | 52 ± 7% | 53 | 8 | 53 ± 8% |
| AD-12802 | 74 | 9 | 74 ± 9% | 29 | 4 | 29 ± 4% |
| AD-12849 | 111 | 5 | 111 ± 5% | −12 | 1 | −12 ± 1% |
| AD-12641 | 103 | 8 | 103 ± 8% | −3 | 0 | −3 ± 0% |
| AD-12803 | 94 | 13 | 94 ± 13% | 6 | 1 | 6 ± 1% |
| AD-12642 | 105 | 3 | 105 ± 3% | −6 | 0 | −6 ± 0% |
| AD-12746 | 100 | 9 | 100 ± 9% | 0 | 0 | 0 ± 0% |
| AD-12643 | 33 | 4 | 33 ± 4% | 74 | 10 | 74 ± 10% |
| AD-12747 | 21 | 3 | 21 ± 3% | 83 | 13 | 83 ± 13% |
| AD-12804 | 25 | 4 | 25 ± 4% | 78 | 14 | 78 ± 14% |
| AD-12850 | 28 | 4 | 28 ± 4% | 75 | 11 | 75 ± 11% |
| AD-12644 | 82 | 7 | 82 ± 7% | 20 | 2 | 20 ± 2% |
| AD-12748 | 25 | 4 | 25 ± 4% | 78 | 14 | 78 ± 14% |
| AD-12805 | 23 | 7 | 23 ± 7% | 80 | 30 | 80 ± 30% |
| AD-12851 | 61 | 7 | 61 ± 7% | 41 | 5 | 41 ± 5% |
| AD-12645 | 112 | 6 | 112 ± 6% | −14 | 1 | −14 ± 1% |
| AD-12749 | 86 | 10 | 86 ± 10% | 16 | 2 | 16 ± 2% |
| AD-12646 | 94 | 10 | 94 ± 10% | 6 | 1 | 6 ± 1% |
| AD-12750 | 93 | 11 | 93 ± 11% | 7 | 1 | 7 ± 1% |
| AD-12806 | 77 | 8 | 77 ± 8% | 24 | 3 | 24 ± 3% |
| AD-12852 | 96 | 4 | 96 ± 4% | 5 | 0 | 5 ± 0% |
| AD-12647 | 27 | 3 | 27 ± 3% | 81 | 11 | 81 ± 11% |
| AD-12751 | 29 | 6 | 29 ± 6% | 74 | 19 | 74 ± 19% |
| AD-12807 | 31 | 2 | 31 ± 2% | 72 | 6 | 72 ± 6% |
| AD-12853 | 26 | 3 | 26 ± 3% | 78 | 11 | 78 ± 11% |
| AD-12648 | 81 | 9 | 81 ± 9% | 17 | 3 | 17 ± 3% |
| AD-12649 | 92 | 9 | 92 ± 9% | 8 | 1 | 8 ± 1% |
| AD-12752 | 71 | 9 | 71 ± 9% | 30 | 5 | 30 ± 5% |
| AD-12650 | 81 | 2 | 81 ± 2% | 21 | 1 | 21 ± 1% |
| AD-12753 | 57 | 1 | 57 ± 1% | 48 | 1 | 48 ± 1% |
| AD-12808 | 52 | 4 | 52 ± 4% | 54 | 5 | 54 ± 5% |
| AD-12854 | 77 | 5 | 77 ± 5% | 26 | 2 | 26 ± 2% |
| AD-12651 | 89 | 6 | 89 ± 6% | 13 | 1 | 13 ± 1% |
| AD-12754 | 88 | 7 | 88 ± 7% | 12 | 1 | 12 ± 1% |
| AD-12809 | 67 | 6 | 67 ± 6% | 35 | 4 | 35 ± 4% |
| AD-12855 | 88 | 10 | 88 ± 10% | 12 | 2 | 12 ± 2% |
| AD-12652 | 91 | 2 | 91 ± 2% | 10 | 0 | 10 ± 0% |
| AD-12755 | 40 | 3 | 40 ± 3% | 67 | 6 | 67 ± 6% |
| AD-12810 | 35 | 1 | 35 ± 1% | 72 | 3 | 72 ± 3% |
| AD-12856 | 75 | 8 | 75 ± 8% | 28 | 4 | 28 ± 4% |
| AD-12653 | 79 | 8 | 79 ± 8% | 23 | 3 | 23 ± 3% |
| AD-12756 | 17 | 5 | 17 ± 5% | 86 | 27 | 86 ± 27% |
| AD-12654 | 97 | 6 | 97 ± 6% | 3 | 0 | 3 ± 0% |
| AD-12811 | 74 | 5 | 74 ± 5% | 27 | 2 | 27 ± 2% |
| AD-12655 | 46 | 6 | 46 ± 6% | 59 | 9 | 59 ± 9% |
| AD-12757 | 14 | 0 | 14 ± 0% | 89 | 2 | 89 ± 2% |
| AD-12812 | 12 | 3 | 12 ± 3% | 92 | 28 | 92 ± 28% |
| AD-12857 | 35 | 7 | 35 ± 7% | 70 | 17 | 70 ± 17% |
| AD-12656 | 10 | 3 | 10 ± 3% | 99 | 33 | 99 ± 33% |
| AD-12813 | 9 | 1 | 9 ± 1% | 95 | 18 | 95 ± 18% |
| AD-12657 | 108 | 1 | 108 ± 1% | −9 | 0 | −9 ± 0% |
| AD-12814 | 101 | 4 | 101 ± 4% | −1 | 0 | −1 ± 0% |
| AD-12658 | 98 | 9 | 98 ± 9% | 2 | 0 | 2 ± 0% |
| AD-12659 | 83 | 4 | 83 ± 4% | 18 | 1 | 18 ± 1% |
| AD-12758 | 80 | 14 | 80 ± 14% | 21 | 4 | 21 ± 4% |
| AD-12815 | 25 | 3 | 25 ± 3% | 79 | 11 | 79 ± 11% |
| AD-12858 | 67 | 4 | 67 ± 4% | 35 | 2 | 35 ± 2% |
| AD-12660 | 95 | 11 | 95 ± 11% | 8 | 3 | 8 ± 3% |
| AD-12759 | 66 | 7 | 66 ± 7% | 39 | 6 | 39 ± 6% |
| AD-12661 | 34 | 2 | 34 ± 2% | 73 | 5 | 73 ± 5% |
| AD-12760 | 10 | 3 | 10 ± 3% | 94 | 30 | 94 ± 30% |
| AD-12816 | 12 | 4 | 12 ± 4% | 92 | 37 | 92 ± 37% |
| AD-12859 | 33 | 1 | 33 ± 1% | 72 | 2 | 72 ± 2% |
| AD-12662 | 92 | 7 | 92 ± 7% | 7 | 1 | 7 ± 1% |
| AD-12817 | 91 | 11 | 91 ± 11% | 10 | 2 | 10 ± 2% |
| AD-12663 | 99 | 10 | 99 ± 10% | 3 | [symbol] | [symbol] |
| AD-12761 | 20 | 5 | 20 ± 5% | 89 | 22 | 89 ± 22% |
| AD-12818 | 20 | 4 | 20 ± 4% | 90 | 20 | 90 ± 20% |
| AD-12860 | 93 | 11 | 93 ± 11% | 8 | 1 | 8 ± 1% |
| AD-12664 | 93 | 9 | 93 ± 9% | 6 | 2 | 6 ± 2% |
| AD-12665 | 94 | 8 | 94 ± 8% | 10 | 1 | 10 ± 1% |
| AD-12762 | 58 | 8 | 58 ± 8% | 47 | 10 | 47 ± 10% |
| AD-12819 | 49 | 6 | 49 ± 6% | 58 | 9 | 58 ± 9% |
| AD-12861 | 93 | 8 | 93 ± 8% | 8 | 1 | 8 ± 1% |
| AD-12666 | 30 | 5 | 30 ± 5% | 76 | 18 | 76 ± 18% |
| AD-12763 | 25 | 2 | 25 ± 2% | 84 | 9 | 84 ± 9% |
| AD-12667 | 65 | 10 | 65 ± 10% | 38 | 7 | 38 ± 7% |
| AD-12764 | 34 | 7 | 34 ± 7% | 69 | 17 | 69 ± 17% |
| AD-12668 | 34 | 4 | 34 ± 4% | 73 | 10 | 73 ± 10% |
| AD-12765 | 13 | 3 | 13 ± 3% | 91 | 22 | 91 ± 22% |
| AD-12820 | 11 | 2 | 11 ± 2% | 93 | 17 | 93 ± 17% |
| AD-12862 | 19 | 4 | 19 ± 4% | 87 | 22 | 87 ± 22% |
| AD-12669 | 22 | 3 | 22 ± 3% | 87 | 12 | 87 ± 12% |
| AD-12766 | 11 | 4 | 11 ± 4% | 93 | 39 | 93 ± 39% |
| AD-12670 | 45 | 3 | 45 ± 3% | 61 | 5 | 61 ± 5% |
| AD-12767 | 10 | 3 | 10 ± 3% | 94 | 31 | 94 ± 31% |
| AD-12821 | 12 | 1 | 12 ± 1% | 92 | 13 | 92 ± 13% |
| AD-12863 | 41 | 4 | 41 ± 4% | 64 | 8 | 64 ± 8% |
| AD-12671 | 83 | 9 | 83 ± 9% | 19 | 2 | 19 ± 2% |
| AD-12822 | 74 | 7 | 74 ± 7% | 29 | 3 | 29 ± 3% |
| AD-12672 | 52 | 7 | 52 ± 7% | 54 | 9 | 54 ± 9% |
| AD-12768 | 28 | 3 | 28 ± 3% | 81 | 12 | 81 ± 12% |
| AD-12673 | 56 | 5 | 56 ± 5% | 49 | 5 | 49 ± 5% |
| AD-12769 | 36 | 2 | 36 ± 2% | 72 | 5 | 72 ± 5% |
| AD-12823 | 33 | 2 | 33 ± 2% | 75 | 5 | 75 ± 5% |
| AD-12864 | 49 | 7 | 49 ± 7% | 57 | 10 | 57 ± 10% |
| AD-12674 | 90 | 9 | 90 ± 9% | 11 | 1 | 11 ± 1% |
| AD-12770 | 45 | 6 | 45 ± 6% | 61 | 9 | 61 ± 9% |
| AD-12675 | 45 | 5 | 45 ± 5% | 62 | 8 | 62 ± 8% |
| AD-12676 | 47 | 6 | 47 ± 6% | 59 | 9 | 59 ± 9% |
| AD-12771 | 31 | 4 | 31 ± 4% | 77 | 11 | 77 ± 11% |
| AD-12824 | 31 | 3 | 31 ± 3% | 77 | 10 | 77 ± 10% |
| AD-12865 | 43 | 7 | 43 ± 7% | 64 | 12 | 64 ± 12% |
| AD-12677 | 23 | 4 | 23 ± 4% | 86 | 16 | 86 ± 16% |
| AD-12825 | 22 | 4 | 22 ± 4% | 87 | 16 | 87 ± 16% |
| AD-12678 | 102 | 8 | 102 ± 8% | −2 | 0 | −2 ± 0% |
| AD-12772 | 101 | 13 | 101 ± 13% | −1 | 0 | −1 ± 0% |
| AD-12826 | 99 | 1 | 99 ± 1% | 1 | 0 | 1 ± 0% |
| AD-12866 | 91 | 7 | 91 ± 7% | 10 | 1 | 10 ± 1% |
| AD-12679 | 81 | 8 | 81 ± 8% | 21 | 2 | 21 ± 2% |
| AD-12773 | 11 | 2 | 11 ± 2% | 93 | 19 | 93 ± 19% |
| AD-12680 | 17 | 3 | 17 ± 3% | 92 | 17 | 92 ± 17% |
| AD-12774 | 15 | 2 | 15 ± 2% | 89 | [symbol] | [symbol] |
| AD-12827 | 11 | 2 | 11 ± 2% | 93 | 18 | 93 ± 18% |
| AD-12867 | 15 | 3 | 15 ± 3% | 91 | 22 | 91 ± 22% |
| AD-12681 | 28 | 3 | 28 ± 3% | 79 | 10 | 79 ± 10% |
| AD-12775 | 8 | 1 | 8 ± 1% | 95 | 19 | 95 ± 19% |
| AD-12682 | 43 | 6 | 43 ± 6% | 63 | 9 | 63 ± 9% |
| AD-12776 | 23 | 5 | 23 ± 5% | 80 | 19 | 80 ± 19% |
| AD-12828 | 23 | 5 | 23 ± 5% | 80 | 20 | 80 ± 20% |
| AD-12868 | 25 | 4 | 25 ± 4% | 81 | 16 | 81 ± 16% |
| AD-12683 | 17 | 2 | 17 ± 2% | 91 | 15 | 91 ± 15% |
| AD-12777 | 11 | 2 | 11 ± 2% | 92 | 22 | 92 ± 22% |
| AD-12829 | 12 | 1 | 12 ± 1% | 92 | 11 | 92 ± 11% |
| AD-12869 | 19 | 3 | 19 ± 3% | 87 | 16 | 87 ± 16% |
| AD-12684 | 87 | 12 | 87 ± 12% | 14 | 2 | 14 ± 2% |
| AD-12778 | 41 | 4 | 41 ± 4% | 66 | 8 | 66 ± 8% |
| AD-12685 | 35 | 1 | 35 ± 1% | 72 | 1 | 72 ± 1% |
| AD-12686 | 68 | 5 | 68 ± 5% | 36 | 3 | 36 ± 3% |
| AD-12779 | 58 | 5 | 58 ± 5% | 47 | 5 | 47 ± 5% |
| AD-12687 | 73 | 8 | 73 ± 8% | 30 | 4 | 30 ± 4% |
| AD-12780 | 62 | 8 | 62 ± 8% | 42 | 7 | 42 ± 7% |
| AD-12688 | 18 | 1 | 18 ± 1% | 91 | 4 | 91 ± 4% |
| AD-12781 | 11 | 3 | 11 ± 3% | 93 | 33 | 93 ± 33% |
| AD-12689 | 96 | 4 | 96 ± 4% | 4 | 0 | 4 ± 0% |
| AD-12782 | 45 | 7 | 45 ± 7% | 58 | 10 | 58 ± 10% |
| AD-12830 | 15 | 3 | 15 ± 3% | 89 | 19 | 89 ± 19% |
| AD-12870 | 51 | 3 | 51 ± 3% | 52 | 4 | 52 ± 4% |
| AD-12690 | 93 | 6 | 93 ± 6% | 8 | 1 | 8 ± 1% |
| AD-12783 | 36 | 3 | 36 ± 3% | 66 | 7 | 66 ± 7% |
| AD-12831 | 27 | 2 | 27 ± 2% | 76 | 7 | 76 ± 7% |
| AD-12871 | 81 | 18 | 81 ± 18% | 21 | 5 | 21 ± 5% |

[symbol]

indicates data missing or illegible when filed

TABLE 13
Exemplary JC Virus unmodified dsRNAs

| Position | SEQ | | | |
|---|---|---|---|---|
| in | ID | Sequence | SEQ ID | Sequence |
| Consensus | NO: | (5′--> 3′) | NO: | (5′--> 3′) |

| | | | | |
|---|---|---|---|---|
| 1533-1551 | 935 | CUUAUAAGAGGAGGAGUAG | 936 | CUACUCCUCCUCUUAUAAG |
| 1703-1721 | 937 | CAUGCUUCCUUGUUACAGU | 938 | ACUGUAACAAGGAAGCAUG |
| 1439-1457 | 939 | UACGGGACUGUAACACCUG | 940 | CAGGUGUUACAGUCCCGUA |
| 1705-1723 | 941 | UGCUUCCUUGUUACAGUGU | 942 | ACACUGUAACAAGGAAGCA |
| 2064-2082 | 943 | CCUGUUGAAUGUUGGGUUC | 944 | GAACCCAACAUUCAACAGG |
| 2067-2085 | 945 | GUUGAAUGUUGGGUUCCUG | 946 | CAGGAACCCAACAUUCAAC |
| 2071-2089 | 947 | AAUGUUGGGUUCCUGAUCC | 948 | GGAUCAGGAACCCAACAUU |
| 2121-2139 | 949 | ACACUAACAGGAGGAGAAA | 950 | UUUCUCCUCCUGUUAGUGU |
| 1535-1553 | 951 | UAUAAGAGGAGGAGUAGAA | 952 | UUCUACUCCUCCUCUUAUA |
| 1536-1554 | 953 | AUAAGAGGAGGAGUAGAAG | 954 | CUUCUACUCCUCCUCUUAU |
| 1445-1463 | 955 | ACUGUAACACCUGCUCUUG | 956 | CAAGAGCAGGUGUUACAGU |
| 1700-1718 | 957 | GGACAUGCUUCCUUGUUAC | 958 | GUAACAAGGAAGCAUGUCC |
| 1702-1720 | 959 | ACAUGCUUCCUUGUUACAG | 960 | CUGUAACAAGGAAGCAUGU |
| 1704-1722 | 961 | AUGCUUCCUUGUUACAGUG | 962 | CACUGUAACAAGGAAGCAU |
| 2065-2083 | 963 | CUGUUGAAUGUUGGGUUCC | 964 | GGAACCCAACAUUCAACAG |
| 2070-2088 | 965 | GAAUGUUGGGUUCCUGAUC | 966 | GAUCAGGAACCCAACAUUC |
| 1441-1459 | 967 | CGGGACUGUAACACCUGCU | 968 | AGCAGGUGUUACAGUCCCG |
| 1443-1461 | 969 | GGACUGUAACACCUGCUCU | 970 | AGAGCAGGUGUUACAGUCC |
| 1444-1462 | 971 | GACUGUAACACCUGCUCUU | 972 | AAGAGCAGGUGUUACAGUC |
| 1609-1627 | 973 | CUCCAGAAAUGGGUGACCC | 974 | GGGUCACCCAUUUCUGGAG |
| 1537-1555 | 975 | UAAGAGGAGGAGUAGAAGU | 976 | ACUUCUACUCCUCCUCUUA |
| 629-647 | 977 | GAGGCUGCUGCUACUAUAG | 978 | CUAUAGUAGCAGCAGCCUC |
| 656-674 | 979 | AUUGCAUCCCUUGCUACUG | 980 | CAGUAGCAAGGGAUGCAAU |
| 658-676 | 981 | UGCAUCCCUUGCUACUGUA | 982 | UACAGUAGCAAGGGAUGCA |
| 517-535 | 983 | UUGUGUUUUCAGGUUCAUG | 984 | CAUGAACCUGAAAACACAA |
| 559-577 | 985 | GGACCUAGUUGCUACUGUU | 986 | AACAGUAGCAACUAGGUCC |
| 591-609 | 987 | CUGCCACAGGAUUUUCAGU | 988 | ACUGAAAAUCCUGUGGCAG |
| 638-656 | 989 | GCUACUAUAGAAGUUGAAA | 990 | UUUCAACUUCUAUAGUAGC |
| 655-673 | 991 | AAUUGCAUCCCUUGCUACU | 992 | AGUAGCAAGGGAUGCAAUU |
| 561-579 | 993 | ACCUAGUUGCUACUGUUUC | 994 | GAAACAGUAGCAACUAGGU |
| 639-657 | 995 | CUACUAUAGAAGUUGAAAU | 996 | AUUUCAACUUCUAUAGUAG |
| 715-733 | 997 | AGGCCUUACUCCUGAAACA | 998 | UGUUUCAGGAGUAAGGCCU |
| 716-734 | 999 | GGCCUUACUCCUGAAACAU | 1000 | AUGUUUCAGGAGUAAGGCC |
| 326-344 | 1001 | GUAAAACCUGGAGUGGAAC | 1002 | GUUCCACUCCAGGUUUUAC |
| 518-536 | 1003 | UGUGUUUUCAGGUUCAUGG | 1004 | CCAUGAACCUGAAAACACA |
| 520-538 | 1005 | UGUUUUCAGGUUCAUGGGU | 1006 | ACCCAUGAACCUGAAAACA |
| 661-679 | 1007 | AUCCCUUGCUACUGUAGAG | 1008 | CUCUACAGUAGCAAGGGAU |
| 560-578 | 1009 | GACCUAGUUGCUACUGUUU | 1010 | AAACAGUAGCAACUAGGUC |
| 681-699 | 1011 | GGAUUACAAGUACCUCUGA | 1012 | UCAGAGGUACUUGUAAUCC |
| 714-732 | 1013 | UAGGCCUUACUCCUGAAAC | 1014 | GUUUCAGGAGUAAGGCCUA |
| 377-395 | 1015 | UGUUAGAAUUUUUGCUGGA | 1016 | UCCAGCAAAAAUUCUAACA |
| 589-607 | 1017 | UGCUGCCACAGGAUUUUCA | 1018 | UGAAAAUCCUGUGGCAGCA |
| 594-612 | 1019 | CCACAGGAUUUUCAGUAGC | 1020 | GCUACUGAAAAUCCUGUGG |
| 648-666 | 1021 | AAGUUGAAAUUGCAUCCCU | 1022 | AGGGAUGCAAUUUCAACUU |
| 649-667 | 1023 | AGUUGAAAUUGCAUCCCUU | 1024 | AAGGGAUGCAAUUUCAACU |
| 587-605 | 1025 | GCUGCUGCCACAGGAUUUU | 1026 | AAAAUCCUGUGGCAGCAGC |
| 325-343 | 1027 | AGUAAAACCUGGAGUGGAA | 1028 | UUCCACUCCAGGUUUUACU |
| 515-533 | 1029 | UUUUGUGUUUUCAGGUUCA | 1030 | UGAACCUGAAAACACAAAA |
| 516-534 | 1031 | UUUGUGUUUUCAGGUUCAU | 1032 | AUGAACCUGAAAACACAAA |
| 519-537 | 1033 | GUGUUUUCAGGUUCAUGGG | 1034 | CCCAUGAACCUGAAAACAC |
| 521-539 | 1035 | GUUUUCAGGUUCAUGGGUG | 1036 | CACCCAUGAACCUGAAAAC |
| 522-540 | 1037 | UUUUCAGGUUCAUGGGUGC | 1038 | GCACCCAUGAACCUGAAAA |
| 523-541 | 1039 | UUUCAGGUUCAUGGGUGCC | 1040 | GGCACCCAUGAACCUGAAA |
| 616-634 | 1041 | AAUUGCUGCUGGAGAGGCU | 1042 | AGCCUCUCCAGCAGCAAUU |
| 657-675 | 1043 | UUGCAUCCCUUGCUACUGU | 1044 | ACAGUAGCAAGGGAUGCAA |
| 761-779 | 1045 | GCUGUAGCUGGGUUUGCUG | 1046 | CAGCAAACCCAGCUACAGC |
| 645-663 | 1047 | UAGAAGUUGAAAUUGCAUC | 1048 | GAUGCAAUUUCAACUUCUA |
| 647-665 | 1049 | GAAGUUGAAAUUGCAUCCC | 1050 | GGGAUGCAAUUUCAACUUC |
| 660-678 | 1051 | CAUCCCUUGCUACUGUAGA | 1052 | UCUACAGUAGCAAGGGAUG |
| 324-342 | 1053 | UAGUAAAACCUGGAGUGGA | 1054 | UCCACUCCAGGUUUUACUA |
| 372-390 | 1055 | UUUUUUGUUAGAAUUUUUG | 1056 | CAAAAAUUCUAACAAAAAA |
| 640-658 | 1057 | UACUAUAGAAGUUGAAAUU | 1058 | AAUUUCAACUUCUAUAGUA |
| 562-580 | 1059 | CCUAGUUGCUACUGUUUCU | 1060 | AGAAACAGUAGCAACUAGG |
| 563-581 | 1061 | CUAGUUGCUACUGUUUCUG | 1062 | CAGAAACAGUAGCAACUAG |
| 566-584 | 1063 | GUUGCUACUGUUUCUGAGG | 1064 | CCUCAGAAACAGUAGCAAC |
| 625-643 | 1065 | UGGAGAGGCUGCUGCUACU | 1066 | AGUAGCAGCAGCCUCUCCA |
| 627-645 | 1067 | GAGAGGCUGCUGCUACUAU | 1068 | AUAGUAGCAGCAGCCUCUC |
| 628-646 | 1069 | AGAGGCUGCUGCUACUAUA | 1070 | UAUAGUAGCAGCAGCCUCU |
| 632-650 | 1071 | GCUGCUGCUACUAUAGAAG | 1072 | CUUCUAUAGUAGCAGCAGC |
| 513-531 | 1073 | UUUUUUGUGUUUUCAGGUU | 1074 | AACCUGAAAACACAAAAAA |
| 641-659 | 1075 | ACUAUAGAAGUUGAAAUUG | 1076 | CAAUUUCAACUUCUAUAGU |
| 323-341 | 1077 | UUAGUAAAACCUGGAGUGG | 1078 | CCACUCCAGGUUUUACUAA |
| 717-735 | 1079 | GCCUUACUCCUGAAACAUA | 1080 | UAUGUUUCAGGAGUAAGGC |
| 646-664 | 1081 | AGAAGUUGAAAUUGCAUCC | 1082 | GGAUGCAAUUUCAACUUCU |
| 592-610 | 1083 | UGCCACAGGAUUUUCAGUA | 1084 | UACUGAAAAUCCUGUGGCA |
| 590-608 | 1085 | GCUGCCACAGGAUUUUCAG | 1086 | CUGAAAAUCCUGUGGCAGC |
| 526-544 | 1087 | CAGGUUCAUGGGUGCCGCA | 1088 | UGCGGCACCCAUGAACCUG |
| 615-633 | 1089 | AAAUUGCUGCUGGAGAGGC | 1090 | GCCUCUCCAGCAGCAAUUU |
| 617-635 | 1091 | AUUGCUGCUGGAGAGGCUG | 1092 | CAGCCUCUCCAGCAGCAAU |
| 652-670 | 1093 | UGAAAUUGCAUCCCUUGCU | 1094 | AGCAAGGGAUGCAAUUUCA |
| 374-392 | 1095 | UUUUGUUAGAAUUUUUGCU | 1096 | AGCAAAAAUUCUAACAAAA |
| 375-393 | 1097 | UUUGUUAGAAUUUUUGCUG | 1098 | CAGCAAAAAUUCUAACAAA |
| 631-649 | 1099 | GGCUGCUGCUACUAUAGAA | 1100 | UUCUAUAGUAGCAGCAGCC |
| 376-394 | 1101 | UUGUUAGAAUUUUUGCUGG | 1102 | CCAGCAAAAAUUCUAACAA |
| 512-530 | 1103 | UUUUUUUGUGUUUUCAGGU | 1104 | ACCUGAAAACACAAAAAAA |
| 1127-1145 | 1105 | GAAACUACUUGGGCAAUAG | 1106 | CUAUUGCCCAAGUAGUUUC |
| 1410-1428 | 1107 | AAUGGAUGUUGCCUUUACU | 1108 | AGUAAAGGCAACAUCCAUU |
| 1406-1424 | 1109 | CCUCAAUGGAUGUUGCCUU | 1110 | AAGGCAACAUCCAUUGAGG |
| 1418-1436 | 1111 | UUGCCUUUACUUUUAGGGU | 1112 | ACCCUAAAAGUAAAGGCAA |
| 1126-1144 | 1113 | AGAAACUACUUGGGCAAUA | 1114 | UAUUGCCCAAGUAGUUUCU |
| 1125-1143 | 1115 | AAGAAACUACUUGGGCAAU | 1116 | AUUGCCCAAGUAGUUUCUU |
| 1419-1437 | 1117 | UGCCUUUACUUUUAGGGUU | 1118 | AACCCUAAAAGUAAAGGCA |
| 1420-1438 | 1119 | GCCUUUACUUUUAGGGUUG | 1120 | CAACCCUAAAAGUAAAGGC |
| 1422-1440 | 1121 | CUUUACUUUUAGGGUUGUA | 1122 | UACAACCCUAAAAGUAAAG |
| 1423-1441 | 1123 | UUUACUUUUAGGGUUGUAC | 1124 | GUACAACCCUAAAAGUAAA |
| 1425-1443 | 1125 | UACUUUUAGGGUUGUACGG | 1126 | CCGUACAACCCUAAAAGUA |
| 1123-1141 | 1127 | GGAAGAAACUACUUGGGCA | 1128 | UGCCCAAGUAGUUUCUUCC |
| 1409-1427 | 1129 | CAAUGGAUGUUGCCUUUAC | 1130 | GUAAAGGCAACAUCCAUUG |
| 1413-1431 | 1131 | GGAUGUUGCCUUUACUUUU | 1132 | AAAAGUAAAGGCAACAUCC |
| 1416-1434 | 1133 | UGUUGCCUUUACUUUUAGG | 1134 | CCUAAAAGUAAAGGCAACA |
| 1414-1432 | 1135 | GAUGUUGCCUUUACUUUUA | 1136 | UAAAAGUAAAGGCAACAUC |
| 911-929 | 1137 | CCAGAAGACUACUAUGAUA | 1138 | UAUCAUAGUAGUCUUCUGG |
| 910-928 | 1139 | UCCAGAAGACUACUAUGAU | 1140 | AUCAUAGUAGUCUUCUGGA |
| 1120-1138 | 1141 | UUUGGAAGAAACUACUUGG | 1142 | CCAAGUAGUUUCUUCCAAA |
| 1404-1422 | 1143 | CUCCUCAAUGGAUGUUGCC | 1144 | GGCAACAUCCAUUGAGGAG |
| 1337-1355 | 1145 | CCAAAUGUGCAAUCUGGUG | 1146 | CACCAGAUUGCACAUUUGG |
| 1338-1356 | 1147 | CAAAUGUGCAAUCUGGUGA | 1148 | UCACCAGAUUGCACAUUUG |
| 1397-1415 | 1149 | AGAUCUGCUCCUCAAUGGA | 1150 | UCCAUUGAGGAGCAGAUCU |
| 1407-1425 | 1151 | CUCAAUGGAUGUUGCCUUU | 1152 | AAAGGCAACAUCCAUUGAG |
| 4157-4175 | 1153 | GCUCAAAUUUUAUAUAAGA | 1154 | UCUUAUAUAAAAUUUGAGC |
| 4795-4813 | 1155 | AGCCUGAUUUUGGUACAUG | 1156 | CAUGUACCAAAAUCAGGCU |
| 4156-4174 | 1157 | CUCAAAUUUUAUAUAAGAA | 1158 | UUCUUAUAUAAAAUUUGAG |
| 5002-5020 | 1159 | ACAAAGUGCUGAAUAGGGA | 1160 | UCCCUAUUCAGCACUUUGU |
| 4792-4810 | 1161 | CUGAUUUUGGUACAUGGAA | 1162 | UUCCAUGUACCAAAAUCAG |
| 4790-4808 | 1163 | GAUUUUGGUACAUGGAAUA | 1164 | UAUUCCAUGUACCAAAAUC |
| 4801-4819 | 1165 | CUCAUCAGCCUGAUUUUGG | 1166 | CCAAAAUCAGGCUGAUGAG |
| 4622-4640 | 1167 | AGCCCACUUGUGUGGAUAG | 1168 | CUAUCCACACAAGUGGGCU |
| 4997-5015 | 1169 | GUGCUGAAUAGGGAGGAAU | 1170 | AUUCCUCCCUAUUCAGCAC |
| 5094-5112 | 1171 | AGUAAGGGCGUGGAGGCUU | 1172 | AAGCCUCCACGCCCUUACU |
| 4564-4582 | 1173 | GUGACUUAACCCAAGAAGC | 1174 | GCUUCUUGGGUUAAGUCAC |
| 5095-5113 | 1175 | UAGUAAGGGCGUGGAGGCU | 1176 | AGCCUCCACGCCCUUACUA |
| 4800-4818 | 1177 | UCAUCAGCCUGAUUUUGGU | 1178 | ACCAAAAUCAGGCUGAUGA |
| 4265-4283 | 1179 | GUAGAAGACCCUAAAGACU | 1180 | AGUCUUUAGGGUCUUCUAC |
| 4267-4285 | 1181 | AGGUAGAAGACCCUAAAGA | 1182 | UCUUUAGGGUCUUCUACCU |
| 4270-4288 | 1183 | AAAAGGUAGAAGACCCUAA | 1184 | UUAGGGUCUUCUACCUUUU |
| 4269-4287 | 1185 | AAAGGUAGAAGACCCUAAA | 1186 | UUUAGGGUCUUCUACCUUU |
| 2874-2892 | 1187 | GAUUGUGCAGUGGAAAGAA | 1188 | UUCUUUCCACUGCACAAUC |
| 2875-2893 | 1189 | GGAUUGUGCAGUGGAAAGA | 1190 | UCUUUCCACUGCACAAUCC |
| 3950-3968 | 1191 | UGUAGACAGCCAUAUGCAG | 1192 | CUGCAUAUGGCUGUCUACA |
| 3896-3914 | 1193 | CAUGACUUUAACCCAGAAG | 1194 | CUUCUGGGUUAAAGUCAUG |
| 4990-5008 | 1195 | AUAGGGAGGAAUCCAUGGA | 1196 | UCCAUGGAUUCCUCCCUAU |
| 4994-5012 | 1197 | CUGAAUAGGGAGGAAUCCA | 1198 | UGGAUUCCUCCCUAUUCAG |
| 5000-5018 | 1199 | AAAGUGCUGAAUAGGGAGG | 1200 | CCUCCCUAUUCAGCACUUU |
| 4563-4581 | 1201 | UGACUUAACCCAAGAAGCU | 1202 | AGCUUCUUGGGUUAAGUCA |
| 3895-3913 | 1203 | AUGACUUUAACCCAGAAGA | 1204 | UCUUCUGGGUUAAAGUCAU |
| 4262-4280 | 1205 | GAAGACCCUAAAGACUUUC | 1206 | GAAAGUCUUUAGGGUCUUC |
| 4162-4180 | 1207 | AAAAAGCUCAAAUUUUAUA | 1208 | UAUAAAAUUUGAGCUUUUU |
| 4798-4816 | 1209 | AUCAGCCUGAUUUUGGUAC | 1210 | GUACCAAAAUCAGGCUGAU |
| 4799-4817 | 1211 | CAUCAGCCUGAUUUUGGUA | 1212 | UACCAAAAUCAGGCUGAUG |
| 5006-5024 | 1213 | AUGGACAAAGUGCUGAAUA | 1214 | UAUUCAGCACUUUGUCCAU |
| 4264-4282 | 1215 | UAGAAGACCCUAAAGACUU | 1216 | AAGUCUUUAGGGUCUUCUA |
| 4268-4286 | 1217 | AAGGUAGAAGACCCUAAAG | 1218 | CUUUAGGGUCUUCUACCUU |
| 4623-4641 | 1219 | CAGCCCACUUGUGUGGAUA | 1220 | UAUCCACACAAGUGGGCUG |
| 4788-4806 | 1221 | UUUUGGUACAUGGAAUAGU | 1222 | ACUAUUCCAUGUACCAAAA |
| 4993-5011 | 1223 | UGAAUAGGGAGGAAUCCAU | 1224 | AUGGAUUCCUCCCUAUUCA |
| 4995-5013 | 1225 | GCUGAAUAGGGAGGAAUCC | 1226 | GGAUUCCUCCCUAUUCAGC |
| 4996-5014 | 1227 | UGCUGAAUAGGGAGGAAUC | 1228 | GAUUCCUCCCUAUUCAGCA |
| 3952-3970 | 1229 | UGUGUAGACAGCCAUAUGC | 1230 | GCAUAUGGCUGUCUACACA |
| 4595-4613 | 1231 | UGCUUUGAUUGCUUCAGAC | 1232 | GUCUGAAGCAAUCAAAGCA |
| 4596-4614 | 1233 | UUGCUUUGAUUGCUUCAGA | 1234 | UCUGAAGCAAUCAAAGCAA |
| 4597-4615 | 1235 | AUUGCUUUGAUUGCUUCAG | 1236 | CUGAAGCAAUCAAAGCAAU |
| 4599-4617 | 1237 | CUAUUGCUUUGAUUGCUUC | 1238 | GAAGCAAUCAAAGCAAUAG |
| 4726-4744 | 1239 | AUUGCAAGGAAUGGCCUAA | 1240 | UUAGGCCAUUCCUUGCAAU |
| 4753-4771 | 1241 | AUUUUCCUCCUAAUUCUGA | 1242 | UCAGAAUUAGGAGGAAAAU |
| 4802-4820 | 1243 | GCUCAUCAGCCUGAUUUUG | 1244 | CAAAAUCAGGCUGAUGAGC |
| 4803-4821 | 1245 | UGCUCAUCAGCCUGAUUUU | 1246 | AAAAUCAGGCUGAUGAGCA |
| 4806-4824 | 1247 | AGUUGCUCAUCAGCCUGAU | 1248 | AUCAGGCUGAUGAGCAACU |
| 5091-5109 | 1249 | AAGGGCGUGGAGGCUUUUU | 1250 | AAAAAGCCUCCACGCCCUU |
| 5093-5111 | 1251 | GUAAGGGCGUGGAGGCUUU | 1252 | AAAGCCUCCACGCCCUUAC |
| 4259-4277 | 1253 | GACCCUAAAGACUUUCCUG | 1254 | CAGGAAAGUCUUUAGGGUC |
| 3901-3919 | 1255 | AGGAGCAUGACUUUAACCC | 1256 | GGGUUAAAGUCAUGCUCCU |
| 4757-4775 | 1257 | UGUGAUUUUCCUCCUAAUU | 1258 | AAUUAGGAGGAAAAUCACA |
| 4758-4776 | 1259 | UUGUGAUUUUCCUCCUAAU | 1260 | AUUAGGAGGAAAAUCACAA |
| 4562-4580 | 1261 | GACUUAACCCAAGAAGCUC | 1262 | GAGCUUCUUGGGUUAAGUC |
| 4585-4603 | 1263 | GCUUCAGACAAUGGUUUGG | 1264 | CCAAACCAUUGUCUGAAGC |
| 4587-4605 | 1265 | UUGCUUCAGACAAUGGUUU | 1266 | AAACCAUUGUCUGAAGCAA |
| 4588-4606 | 1267 | AUUGCUUCAGACAAUGGUU | 1268 | AACCAUUGUCUGAAGCAAU |
| 4591-4609 | 1269 | UUGAUUGCUUCAGACAAUG | 1270 | CAUUGUCUGAAGCAAUCAA |
| 5003-5021 | 1271 | GACAAAGUGCUGAAUAGGG | 1272 | CCCUAUUCAGCACUUUGUC |
| 4165-4183 | 1273 | AAGAAAAAGCUCAAAUUUU | 1274 | AAAAUUUGAGCUUUUUCUU |
| 4166-4184 | 1275 | AAAGAAAAAGCUCAAAUUU | 1276 | AAAUUUGAGCUUUUUCUUU |
| 4263-4281 | 1277 | AGAAGACCCUAAAGACUUU | 1278 | AAAGUCUUUAGGGUCUUCU |
| 4274-4292 | 1279 | AAAAAAAAGGUAGAAGACC | 1280 | GGUCUUCUACCUUUUUUUU |
| 4266-4284 | 1281 | GGUAGAAGACCCUAAAGAC | 1282 | GUCUUUAGGGUCUUCUACC |
| 4272-4290 | 1283 | AAAAAAGGUAGAAGACCCU | 1284 | AGGGUCUUCUACCUUUUUU |
| 4271-4289 | 1285 | AAAAAGGUAGAAGACCCUA | 1286 | UAGGGUCUUCUACCUUUUU |
| 4559-4577 | 1287 | UUAACCCAAGAAGCUCUUC | 1288 | GAAGAGCUUCUUGGGUUAA |
| 4789-4807 | 1289 | AUUUUGGUACAUGGAAUAG | 1290 | CUAUUCCAUGUACCAAAAU |
| 4998-5016 | 1291 | AGUGCUGAAUAGGGAGGAA | 1292 | UUCCUCCCUAUUCAGCACU |
| 5070-5088 | 1293 | GAGGCCAGGGAAAUUCCCU | 1294 | AGGGAAUUUCCCUGGCCUC |
| 4158-4176 | 1295 | AGCUCAAAUUUUAUAUAAG | 1296 | CUUAUAUAAAAUUUGAGCU |
| 5065-5083 | 1297 | CAGGGAAAUUCCCUUGUUU | 1298 | AAACAAGGGAAUUUCCCUG |
| 2872-2890 | 1299 | UUGUGCAGUGGAAAGAAAG | 1300 | CUUUCUUUCCACUGCACAA |
| 4782-4800 | 1301 | UACAUGGAAUAGUUCAGAG | 1302 | CUCUGAACUAUUCCAUGUA |
| 4783-4801 | 1303 | GUACAUGGAAUAGUUCAGA | 1304 | UCUGAACUAUUCCAUGUAC |
| 5064-5082 | 1305 | AGGGAAAUUCCCUUGUUUU | 1306 | AAAACAAGGGAAUUUCCCU |
| 5071-5089 | 1307 | GGAGGCCAGGGAAAUUCCC | 1308 | GGGAAUUUCCCUGGCCUCC |
| 3951-3969 | 1309 | GUGUAGACAGCCAUAUGCA | 1310 | UGCAUAUGGCUGUCUACAC |
| 3949-3967 | 1311 | GUAGACAGCCAUAUGCAGU | 1312 | ACUGCAUAUGGCUGUCUAC |
| 4355-4373 | 1313 | GAAGACCUGUUUUGCCAUG | 1314 | CAUGGCAAAACAGGUCUUC |
| 4363-4381 | 1315 | AGUGGGAUGAAGACCUGUU | 1316 | AACAGGUCUUCAUCCCACU |
| 4356-4374 | 1317 | UGAAGACCUGUUUUGCCAU | 1318 | AUGGCAAAACAGGUCUUCA |
| 4361-4379 | 1319 | UGGGAUGAAGACCUGUUUU | 1320 | AAAACAGGUCUUCAUCCCA |
| 4560-4578 | 1321 | CUUAACCCAAGAAGCUCUU | 1322 | AAGAGCUUCUUGGGUUAAG |
| 2873-2891 | 1323 | AUUGUGCAGUGGAAAGAAA | 1324 | UUUCUUUCCACUGCACAAU |
| 4730-4748 | 1325 | CUUUAUUGCAAGGAAUGGC | 1326 | GCCAUUCCUUGCAAUAAAG |
| 3899-3917 | 1327 | GAGCAUGACUUUAACCCAG | 1328 | CUGGGUUAAAGUCAUGCUC |
| 4756-4774 | 1329 | GUGAUUUUCCUCCUAAUUC | 1330 | GAAUUAGGAGGAAAAUCAC |
| 4590-4608 | 1331 | UGAUUGCUUCAGACAAUGG | 1332 | CCAUUGUCUGAAGCAAUCA |
| 4159-4177 | 1333 | AAGCUCAAAUUUUAUAUAA | 1334 | UUAUAUAAAAUUUGAGCUU |
| 2743-2761 | 1335 | CUGGACAUGGAUCAAGCAC | 1336 | GUGCUUGAUCCAUGUCCAG |
| 4155-4173 | 1337 | UCAAAUUUUAUAUAAGAAA | 1338 | UUUCUUAUAUAAAAUUUGA |
| 2871-2889 | 1339 | UGUGCAGUGGAAAGAAAGG | 1340 | CCUUUCUUUCCACUGCACA |
| 4786-4804 | 1341 | UUGGUACAUGGAAUAGUUC | 1342 | GAACUAUUCCAUGUACCAA |
| 4364-4382 | 1343 | AAGUGGGAUGAAGACCUGU | 1344 | ACAGGUCUUCAUCCCACUU |
| 4359-4377 | 1345 | GGAUGAAGACCUGUUUUGC | 1346 | GCAAAACAGGUCUUCAUCC |
| 2744-2762 | 1347 | UCUGGACAUGGAUCAAGCA | 1348 | UGCUUGAUCCAUGUCCAGA |
| 4787-4805 | 1349 | UUUGGUACAUGGAAUAGUU | 1350 | AACUAUUCCAUGUACCAAA |

TABLE 14
Exemplary JC Virus unmodified dsRNAs

| Position | SEQ | | SEQ | |
|---|---|---|---|---|
| in | ID | Sequence | ID | Sequence |
| Consensus | NO: | (5′--> 3′) | NO: | (5′--> 3′) |
| 1426-1444 | 1351 | ACUUUUAGGGUUGUACGGG | 1352 | CCCGUACAACCCUAAAAGU |
| 1427-1445 | 1353 | CUUUUAGGGUUGUACGGGA | 1354 | UCCCGUACAACCCUAAAAG |
| 2026-2044 | 1355 | CAGAGCACAAGGCGUACCU | 1356 | AGGUACGCCUUGUGCUCUG |
| 1431-1449 | 1357 | UAGGGUUGUACGGGACUG | 1358 | ACAGUCCCGUACAACCCUA |
| 1432-1450 | 1359 | AGGGUUGUACGGGACUGUA | 1360 | UACAGUCCCGUACAACCCU |
| 1436-1454 | 1361 | UUGUACGGGACUGUAACAC | 1362 | GUGUUACAGUCCCGUACAA |
| 4794-4812 | 1363 | GCCUGAUUUUGGUACAUGG | 1364 | CCAUGUACCAAAAUCAGGC |
| 5099-5117 | 1365 | GAAGUAGUAAGGGCGUGGA | 1366 | UCCACGCCCUUACUACUUC |
| 713-731 | 1367 | AUAGGCCUUACUCCUGAAA | 1368 | UUUCAGGAGUAAGGCCUAU |
| 3946-3964 | 1369 | GACAGCCAUAUGCAGUAGU | 1370 | ACUACUGCAUAUGGCUGUC |
| 1128-1146 | 1371 | AAACUACUUGGGCAAUAGU | 1372 | ACUAUUGCCCAAGUAGUUU |
| 525-543 | 1373 | UCAGGUUCAUGGGUGCCGC | 1374 | GCGGCACCCAUGAACCUGA |
| 5096-5114 | 1375 | GUAGUAAGGGCGUGGAGGC | 1376 | GCCUCCACGCCCUUACUAC |
| 4727-4745 | 1377 | UAUUGCAAGGAAUGGCCUA | 1378 | UAGGCCAUUCCUUGCAAUA |
| 5097-5115 | 1379 | AGUAGUAAGGGCGUGGAGG | 1380 | CCUCCACGCCCUUACUACU |
| 4601-4619 | 1381 | UGCUAUUGCUUUGAUUGCU | 1382 | AGCAAUCAAAGCAAUAGCA |
| 4600-4618 | 1383 | GCUAUUGCUUUGAUUGCUU | 1384 | AAGCAAUCAAAGCAAUAGC |
| 1421-1439 | 1385 | CCUUUACUUUUAGGGUUGU | 1386 | ACAACCCUAAAAGUAAAGG |
| 1424-1442 | 1387 | UUACUUUUAGGGUUGUACG | 1388 | CGUACAACCCUAAAAGUAA |
| 1403-1421 | 1389 | GCUCCUCAAUGGAUGUUGC | 1390 | GCAACAUCCAUUGAGGAGC |
| 1534-1552 | 1391 | UUAUAAGAGGAGGAGUAGA | 1392 | UCUACUCCUCCUCUUAUAA |
| 5098-5116 | 1393 | AAGUAGUAAGGGCGUGGAG | 1394 | CUCCACGCCCUUACUACUU |
| 1430-1448 | 1395 | UUAGGGUUGUACGGGACUG | 1396 | CAGUCCCGUACAACCCUAA |
| 1701-1719 | 1397 | GACAUGCUUCCUUGUUACA | 1398 | UGUAACAAGGAAGCAUGUC |
| 2066-2084 | 1399 | UGUUGAAUGUUGGGUUCCU | 1400 | AGGAACCCAACAUUCAACA |
| 4561-4579 | 1401 | ACUUAACCCAAGAAGCUCUT | 1402 | AGAGCUUCUUGGGUUAAGU |
| 4797-4815 | 1403 | UCAGCCUGAUUUUGGUACA | 1404 | UGUACCAAAAUCAGGCUGA |
| 1428-1446 | 1405 | UUUUAGGGUUGUACGGGAC | 1406 | GUCCCGUACAACCCUAAAA |
| 1429-1447 | 1407 | UUUAGGGUUGUACGGGACU | 1408 | AGUCCCGUACAACCCUAAA |
| 662-680 | 1409 | UCCCUUGCUACUGUAGAGG | 1410 | CCUCUACAGUAGCAAGGGA |
| 663-681 | 1411 | CCCUUGCUACUGUAGAGGG | 1412 | CCCUCUACAGUAGCAAGGG |
| 1402-1420 | 1413 | UGCUCCUCAAUGGAUGUUG | 1414 | CAACAUCCAUUGAGGAGCA |
| 1398-1416 | 1415 | GAUCUGCUCCUCAAUGGAU | 1416 | AUCCAUUGAGGAGCAGAUC |
| 1399-1417 | 1417 | AUCUGCUCCUCAAUGGAUG | 1418 | CAUCCAUUGAGGAGCAGAU |
| 1400-1418 | 1419 | UCUGCUCCUCAAUGGAUGU | 1420 | ACAUCCAUUGAGGAGCAGA |
| 1401-1419 | 1421 | CUGCUCCUCAAUGGAUGUU | 1422 | AACAUCCAUUGAGGAGCAG |
| 1435-1453 | 1423 | GUUGUACGGGACUGUAACA | 1424 | UGUUACAGUCCCGUACAAC |
| 1437-1455 | 1425 | UGUACGGGACUGUAACACC | 1426 | GGUGUUACAGUCCCGUACA |
| 1438-1456 | 1427 | GUACGGGACUGUAACACCU | 1428 | AGGUGUUACAGUCCCGUAC |
| 4796-4814 | 1429 | CAGCCUGAUUUUGGUACAU | 1430 | AUGUACCAAAAUCAGGCUG |
| 4992-5010 | 1431 | GAAUAGGGAGGAAUCCAUG | 1432 | CAUGGAUUCCUCCCUAUUC |
| 4999-5017 | 1433 | AAGUGCUGAAUAGGGAGGA | 1434 | UCCUCCCUAUUCAGCACUU |
| 630-648 | 1435 | AGGCUGCUGCUACUAUAGA | 1436 | UCUAUAGUAGCAGCAGCCU |
| 3947-3965 | 1437 | AGACAGCCAUAUGCAGUAG | 1438 | CUACUGCAUAUGGCUGUCU |
| 524-542 | 1439 | UUCAGGUUCAUGGGUGCCG | 1440 | CGGCACCCAUGAACCUGAA |
| 3948-3966 | 1441 | UAGACAGCCAUAUGCAGUA | 1442 | UACUGCAUAUGGCUGUCUA |
| 3900-3918 | 1443 | GGAGCAUGACUUUAACCCA | 1444 | UGGGUUAAAGUCAUGCUCC |
| 1417-1435 | 1445 | GUUGCCUUUACUUUUAGGG | 1446 | CCCUAAAAGUAAAGGCAAC |
| 4565-4583 | 1447 | UGUGACUUAACCCAAGAAG | 1448 | CUUCUUGGGUUAAGUCACA |
| 4598-4616 | 1449 | UAUUGCUUUGAUUGCUUCA | 1450 | UGAAGCAAUCAAAGCAAUA |
| 2060-2078 | 1451 | AUAUCCUGUUGAAUGUUGG | 1452 | CCAACAUUCAACAGGAUAU |
| 4729-4747 | 1453 | UUUAUUGCAAGGAAUGGCC | 1454 | GGCCAUUCCUUGCAAUAAA |
| 1122-1140 | 1455 | UGGAAGAAACUACUUGGGC | 1456 | GCCCAAGUAGUUUCUUCCA |
| 4261-4279 | 1457 | AAGACCCUAAAGACUUUCC | 1458 | GGAAAGUCUUUAGGGUCUU |
| 1412-1430 | 1459 | UGGAUGUUGCCUUUACUUU | 1460 | AAAGUAAAGGCAACAUCCA |
| 4592-4610 | 1461 | UUUGAUUGCUUCAGACAAU | 1462 | AUUGUCUGAAGCAAUCAAA |
| 4991-5009 | 1463 | AAUAGGGAGGAAUCCAUGG | 1464 | CCAUGGAUUCCUCCCUAUU |
| 5004-5022 | 1465 | GGACAAAGUGCUGAAUAGG | 1466 | CCUAUUCAGCACUUUGUCC |
| 5005-5023 | 1467 | UGGACAAAGUGCUGAAUAG | 1468 | CUAUUCAGCACUUUGUCCA |
| 654-672 | 1469 | AAAUUGCAUCCCUUGCUAC | 1470 | GUAGCAAGGGAUGCAAUUU |
| 659-677 | 1471 | GCAUCCCUUGCUACUGUAG | 1472 | CUACAGUAGCAAGGGAUGC |
| 4273-4291 | 1473 | AAAAAAAGGUAGAAGACCC | 1474 | GGGUCUUCUACCUUUUUUU |
| 2025-2043 | 1475 | ACAGAGCACAAGGCGUACC | 1476 | GGUACGCCUUGUGCUCUGU |
| 4791-4809 | 1477 | UGAUUUUGGUACAUGGAAU | 1478 | AUUCCAUGUACCAAAAUCA |
| 1433-1451 | 1479 | GGGUUGUACGGGACUGUAA | 1480 | UUACAGUCCCGUACAACCC |
| 1434-1452 | 1481 | GGUUGUACGGGACUGUAAC | 1482 | GUUACAGUCCCGUACAACC |
| 1440-1458 | 1483 | ACGGGACUGUAACACCUGC | 1484 | GCAGGUGUUACAGUCCCGU |
| 1442-1460 | 1485 | GGGACUGUAACACCUGCUC | 1486 | GAGCAGGUGUUACAGUCCC |
| 1608-1626 | 1487 | ACUCCAGAAAUGGGUGACC | 1488 | GGUCACCCAUUUCUGGAGU |
| 4793-4811 | 1489 | CCUGAUUUUGGUACAUGGA | 1490 | UCCAUGUACCAAAAUCAGG |

TABLE 15
Exemplary JC Virus dsRNAs with NN-dinucleotide overhangs

| Position | SEQ | | | |
|---|---|---|---|---|
| in | ID | Sequence | SEQ ID | Sequence |
| Consensus | NO: | (5′--> 3′) | NO: | (5′--> 3′) |
| 1533-1551 | 1491 | CUUAUAAGAGGAGGAGUAGNN | 1492 | CUACUCCUCCUCUUAUAAGNN |
| 1703-1721 | 1493 | CAUGCUUCCUUGUUACAGUNN | 1494 | ACUGUAACAAGGAAGCAUGNN |
| 1439-1457 | 1495 | UACGGGACUGUAACACCUGNN | 1496 | CAGGUGUUACAGUCCCGUANN |
| 1705-1723 | 1497 | UGCUUCCUUGUUACAGUGUNN | 1498 | ACACUGUAACAAGGAAGCANN |
| 2064-2082 | 1499 | CCUGUUGAAUGUUGGGUUCNN | 1500 | GAACCCAACAUUCAACAGGNN |
| 2067-2085 | 1501 | GUUGAAUGUUGGGUUCCUGNN | 1502 | CAGGAACCCAACAUUCAACNN |
| 2071-2089 | 1503 | AAUGUUGGGUUCCUGAUCCNN | 1504 | GGAUCAGGAACCCAACAUUNN |
| 2121-2139 | 1505 | ACACUAACAGGAGGAGAAANN | 1506 | UUUCUCCUCCUGUUAGUGUNN |
| 1535-1553 | 1507 | UAUAAGAGGAGGAGUAGAANN | 1508 | UUCUACUCCUCCUCUUAUANN |
| 1536-1554 | 1509 | AUAAGAGGAGGAGUAGAAGNN | 1510 | CUUCUACUCCUCCUCUUAUNN |
| 1445-1463 | 1511 | ACUGUAACACCUGCUCUUGNN | 1512 | CAAGAGCAGGUGUUACAGUNN |
| 1700-1718 | 1513 | GGACAUGCUUCCUUGUUACNN | 1514 | GUAACAAGGAAGCAUGUCCNN |
| 1702-1720 | 1515 | ACAUGCUUCCUUGUUACAGNN | 1516 | CUGUAACAAGGAAGCAUGUNN |
| 1704-1722 | 1517 | AUGCUUCCUUGUUACAGUGNN | 1518 | CACUGUAACAAGGAAGCAUNN |
| 2065-2083 | 1519 | CUGUUGAAUGUUGGGUUCCNN | 1520 | GGAACCCAACAUUCAACAGNN |
| 2070-2088 | 1521 | GAAUGUUGGGUUCCUGAUCNN | 1522 | GAUCAGGAACCCAACAUUCNN |
| 1441-1459 | 1523 | CGGGACUGUAACACCUGCUNN | 1524 | AGCAGGUGUUACAGUCCCGNN |
| 1443-1461 | 1525 | GGACUGUAACACCUGCUCUNN | 1526 | AGAGCAGGUGUUACAGUCCNN |
| 1444-1462 | 1527 | GACUGUAACACCUGCUCUUNN | 1528 | AAGAGCAGGUGUUACAGUCNN |
| 1609-1627 | 1529 | CUCCAGAAAUGGGUGACCCNN | 1530 | GGGUCACCCAUUUCUGGAGNN |
| 1537-1555 | 1531 | UAAGAGGAGGAGUAGAAGUNN | 1532 | ACUUCUACUCCUCCUCUUANN |
| 629-647 | 1533 | GAGGCUGCUGCUACUAUAGNN | 1534 | CUAUAGUAGCAGCAGCCUCNN |
| 656-674 | 1535 | AUUGCAUCCCUUGCUACUGNN | 1536 | CAGUAGCAAGGGAUGCAAUNN |
| 658-676 | 1537 | UGCAUCCCUUGCUACUGUANN | 1538 | UACAGUAGCAAGGGAUGCANN |
| 517-535 | 1539 | UUGUGUUUUCAGGUUCAUGNN | 1540 | CAUGAACCUGAAAACACAANN |
| 559-577 | 1541 | GGACCUAGUUGCUACUGUUNN | 1542 | AACAGUAGCAACUAGGUCCNN |
| 591-609 | 1543 | CUGCCACAGGAUUUUCAGUNN | 1544 | ACUGAAAAUCCUGUGGCAGNN |
| 638-656 | 1545 | GCUACUAUAGAAGUUGAAANN | 1546 | UUUCAACUUCUAUAGUAGCNN |
| 655-673 | 1547 | AAUUGCAUCCCUUGCUACUNN | 1548 | AGUAGCAAGGGAUGCAAUUNN |
| 561-579 | 1549 | ACCUAGUUGCUACUGUUUCNN | 1550 | GAAACAGUAGCAACUAGGUNN |
| 639-657 | 1551 | CUACUAUAGAAGUUGAAAUNN | 1552 | AUUUCAACUUCUAUAGUAGNN |
| 715-733 | 1553 | AGGCCUUACUCCUGAAACANN | 1554 | UGUUUCAGGAGUAAGGCCUNN |
| 716-734 | 1555 | GGCCUUACUCCUGAAACAUNN | 1556 | AUGUUUCAGGAGUAAGGCCNN |
| 326-344 | 1557 | GUAAAACCUGGAGUGGAACNN | 1558 | GUUCCACUCCAGGUUUUACNN |
| 518-536 | 1559 | UGUGUUUUCAGGUUCAUGGNN | 1560 | CCAUGAACCUGAAAACACANN |
| 520-538 | 1561 | UGUUUUCAGGUUCAUGGGUNN | 1562 | ACCCAUGAACCUGAAAACANN |
| 661-679 | 1563 | AUCCCUUGCUACUGUAGAGNN | 1564 | CUCUACAGUAGCAAGGGAUNN |
| 560-578 | 1565 | GACCUAGUUGCUACUGUUUNN | 1566 | AAACAGUAGCAACUAGGUCNN |
| 681-699 | 1567 | GGAUUACAAGUACCUCUGANN | 1568 | UCAGAGGUACUUGUAAUCCNN |
| 714-732 | 1569 | UAGGCCUUACUCCUGAAACNN | 1570 | GUUUCAGGAGUAAGGCCUANN |
| 377-395 | 1571 | UGUUAGAAUUUUUGCUGGANN | 1572 | UCCAGCAAAAAUUCUAACANN |
| 589-607 | 1573 | UGCUGCCACAGGAUUUUCANN | 1574 | UGAAAAUCCUGUGGCAGCANN |
| 594-612 | 1575 | CCACAGGAUUUUCAGUAGCNN | 1576 | GCUACUGAAAAUCCUGUGGNN |
| 648-666 | 1577 | AAGUUGAAAUUGCAUCCCUNN | 1578 | AGGGAUGCAAUUUCAACUUNN |
| 649-667 | 1579 | AGUUGAAAUUGCAUCCCUUNN | 1580 | AAGGGAUGCAAUUUCAACUNN |
| 587-605 | 1581 | GCUGCUGCCACAGGAUUUUNN | 1582 | AAAAUCCUGUGGCAGCAGCNN |
| 325-343 | 1583 | AGUAAAACCUGGAGUGGAANN | 1584 | UUCCACUCCAGGUUUUACUNN |
| 515-533 | 1585 | UUUUGUGUUUUCAGGUUCANN | 1586 | UGAACCUGAAAACACAAAANN |
| 516-534 | 1587 | UUUGUGUUUUCAGGUUCAUNN | 1588 | AUGAACCUGAAAACACAAANN |
| 519-537 | 1589 | GUGUUUUCAGGUUCAUGGGNN | 1590 | CCCAUGAACCUGAAAACACNN |
| 521-539 | 1591 | GUUUUCAGGUUCAUGGGUGNN | 1592 | CACCCAUGAACCUGAAAACNN |
| 522-540 | 1593 | UUUUCAGGUUCAUGGGUGCNN | 1594 | GCACCCAUGAACCUGAAAANN |
| 523-541 | 1595 | UUUCAGGUUCAUGGGUGCCNN | 1596 | GGCACCCAUGAACCUGAAANN |
| 616-634 | 1597 | AAUUGCUGCUGGAGAGGCUNN | 1598 | AGCCUCUCCAGCAGCAAUUNN |
| 657-675 | 1599 | UUGCAUCCCUUGCUACUGUNN | 1600 | ACAGUAGCAAGGGAUGCAANN |
| 761-779 | 1601 | GCUGUAGCUGGGUUUGCUGNN | 1602 | CAGCAAACCCAGCUACAGCNN |
| 645-663 | 1603 | UAGAAGUUGAAAUUGCAUCNN | 1604 | GAUGCAAUUUCAACUUCUANN |
| 647-665 | 1605 | GAAGUUGAAAUUGCAUCCCNN | 1606 | GGGAUGCAAUUUCAACUUCNN |
| 660-678 | 1607 | CAUCCCUUGCUACUGUAGANN | 1608 | UCUACAGUAGCAAGGGAUGNN |
| 324-342 | 1609 | UAGUAAAACCUGGAGUGGANN | 1610 | UCCACUCCAGGUUUUACUANN |
| 372-390 | 1611 | UUUUUUGUUAGAAUUUUUGNN | 1612 | CAAAAAUUCUAACAAAAAANN |
| 640-658 | 1613 | UACUAUAGAAGUUGAAAUUNN | 1614 | AAUUUCAACUUCUAUAGUANN |
| 562-580 | 1615 | CCUAGUUGCUACUGUUUCUNN | 1616 | AGAAACAGUAGCAACUAGGNN |
| 563-581 | 1617 | CUAGUUGCUACUGUUUCUGNN | 1618 | CAGAAACAGUAGCAACUAGNN |
| 566-584 | 1619 | GUUGCUACUGUUUCUGAGGNN | 1620 | CCUCAGAAACAGUAGCAACNN |
| 625-643 | 1621 | UGGAGAGGCUGCUGCUACUNN | 1622 | AGUAGCAGCAGCCUCUCCANN |
| 627-645 | 1623 | GAGAGGCUGCUGCUACUAUNN | 1624 | AUAGUAGCAGCAGCCUCUCNN |
| 628-646 | 1625 | AGAGGCUGCUGCUACUAUANN | 1626 | UAUAGUAGCAGCAGCCUCUNN |
| 632-650 | 1627 | GCUGCUGCUACUAUAGAAGNN | 1628 | CUUCUAUAGUAGCAGCAGCNN |
| 513-531 | 1629 | UUUUUUGUGUUUUCAGGUUNN | 1630 | AACCUGAAAACACAAAAAANN |
| 641-659 | 1631 | ACUAUAGAAGUUGAAAUUGNN | 1632 | CAAUUUCAACUUCUAUAGUNN |
| 323-341 | 1633 | UUAGUAAAACCUGGAGUGGNN | 1634 | CCACUCCAGGUUUUACUAANN |
| 717-735 | 1635 | GCCUUACUCCUGAAACAUANN | 1636 | UAUGUUUCAGGAGUAAGGCNN |
| 646-664 | 1637 | AGAAGUUGAAAUUGCAUCCNN | 1638 | GGAUGCAAUUUCAACUUCUNN |
| 592-610 | 1639 | UGCCACAGGAUUUUCAGUANN | 1640 | UACUGAAAAUCCUGUGGCANN |
| 590-608 | 1641 | GCUGCCACAGGAUUUUCAGNN | 1642 | CUGAAAAUCCUGUGGCAGCNN |
| 526-544 | 1643 | CAGGUUCAUGGGUGCCGCANN | 1644 | UGCGGCACCCAUGAACCUGNN |
| 615-633 | 1645 | AAAUUGCUGCUGGAGAGGCNN | 1646 | GCCUCUCCAGCAGCAAUUUNN |
| 617-635 | 1647 | AUUGCUGCUGGAGAGGCUGNN | 1648 | CAGCCUCUCCAGCAGCAAUNN |
| 652-670 | 1649 | UGAAAUUGCAUCCCUUGCUNN | 1650 | AGCAAGGGAUGCAAUUUCANN |
| 374-392 | 1651 | UUUUGUUAGAAUUUUUGCUNN | 1652 | AGCAAAAAUUCUAACAAAANN |
| 375-393 | 1653 | UUUGUUAGAAUUUUUGCUGNN | 1654 | CAGCAAAAAUUCUAACAAANN |
| 631-649 | 1655 | GGCUGCUGCUACUAUAGAANN | 1656 | UUCUAUAGUAGCAGCAGCCNN |
| 376-394 | 1657 | UUGUUAGAAUUUUUGCUGGNN | 1658 | CCAGCAAAAAUUCUAACAANN |
| 512-530 | 1659 | UUUUUUUGUGUUUUCAGGUNN | 1660 | ACCUGAAAACACAAAAAAANN |
| 1127-1145 | 1661 | GAAACUACUUGGGCAAUAGNN | 1662 | CUAUUGCCCAAGUAGUUUCNN |
| 1410-1428 | 1663 | AAUGGAUGUUGCCUUUACUNN | 1664 | AGUAAAGGCAACAUCCAUUNN |
| 1406-1424 | 1665 | CCUCAAUGGAUGUUGCCUUNN | 1666 | AAGGCAACAUCCAUUGAGGNN |
| 1418-1436 | 1667 | UUGCCUUUACUUUUAGGGUNN | 1668 | ACCCUAAAAGUAAAGGCAANN |
| 1126-1144 | 1669 | AGAAACUACUUGGGCAAUANN | 1670 | UAUUGCCCAAGUAGUUUCUNN |
| 1125-1143 | 1671 | AAGAAACUACUUGGGCAAUNN | 1672 | AUUGCCCAAGUAGUUUCUUNN |
| 1419-1437 | 1673 | UGCCUUUACUUUUAGGGUUNN | 1674 | AACCCUAAAAGUAAAGGCANN |
| 1420-1438 | 1675 | GCCUUUACUUUUAGGGUUGNN | 1676 | CAACCCUAAAAGUAAAGGCNN |
| 1422-1440 | 1677 | CUUUACUUUUAGGGUUGUANN | 1678 | UACAACCCUAAAAGUAAAGNN |
| 1423-1441 | 1679 | UUUACUUUUAGGGUUGUACNN | 1680 | GUACAACCCUAAAAGUAAANN |
| 1425-1443 | 1681 | UACUUUUAGGGUUGUACGGNN | 1682 | CCGUACAACCCUAAAAGUANN |
| 1123-1141 | 1683 | GGAAGAAACUACUUGGGCANN | 1684 | UGCCCAAGUAGUUUCUUCCNN |
| 1409-1427 | 1685 | CAAUGGAUGUUGCCUUUACNN | 1686 | GUAAAGGCAACAUCCAUUGNN |
| 1413-1431 | 1687 | GGAUGUUGCCUUUACUUUUNN | 1688 | AAAAGUAAAGGCAACAUCCNN |
| 1416-1434 | 1689 | UGUUGCCUUUACUUUUAGGNN | 1690 | CCUAAAAGUAAAGGCAACANN |
| 1414-1432 | 1691 | GAUGUUGCCUUUACUUUUANN | 1692 | UAAAAGUAAAGGCAACAUCNN |
| 911-929 | 1693 | CCAGAAGACUACUAUGAUANN | 1694 | UAUCAUAGUAGUCUUCUGGNN |
| 910-928 | 1695 | UCCAGAAGACUACUAUGAUNN | 1696 | AUCAUAGUAGUCUUCUGGANN |
| 1120-1138 | 1697 | UUUGGAAGAAACUACUUGGNN | 1698 | CCAAGUAGUUUCUUCCAAANN |
| 1404-1422 | 1699 | CUCCUCAAUGGAUGUUGCCNN | 1700 | GGCAACAUCCAUUGAGGAGNN |
| 1337-1355 | 1701 | CCAAAUGUGCAAUCUGGUGNN | 1702 | CACCAGAUUGCACAUUUGGNN |
| 1338-1356 | 1703 | CAAAUGUGCAAUCUGGUGANN | 1704 | UCACCAGAUUGCACAUUUGNN |
| 1397-1415 | 1705 | AGAUCUGCUCCUCAAUGGANN | 1706 | UCCAUUGAGGAGCAGAUCUNN |
| 1407-1425 | 1707 | CUCAAUGGAUGUUGCCUUUNN | 1708 | AAAGGCAACAUCCAUUGAGNN |
| 4157-4175 | 1709 | GCUCAAAUUUUAUAUAAGANN | 1710 | UCUUAUAUAAAAUUUGAGCNN |
| 4795-4813 | 1711 | AGCCUGAUUUUGGUACAUGNN | 1712 | CAUGUACCAAAAUCAGGCUNN |
| 4156-4174 | 1713 | CUCAAAUUUUAUAUAAGAANN | 1714 | UUCUUAUAUAAAAUUUGAGNN |
| 5002-5020 | 1715 | ACAAAGUGCUGAAUAGGGANN | 1716 | UCCCUAUUCAGCACUUUGUNN |
| 4792-4810 | 1717 | CUGAUUUUGGUACAUGGAANN | 1718 | UUCCAUGUACCAAAAUCAGNN |
| 4790-4808 | 1719 | GAUUUUGGUACAUGGAAUANN | 1720 | UAUUCCAUGUACCAAAAUCNN |
| 4801-4819 | 1721 | CUCAUCAGCCUGAUUUUGGNN | 1722 | CCAAAAUCAGGCUGAUGAGNN |
| 4622-4640 | 1723 | AGCCCACUUGUGUGGAUAGNN | 1724 | CUAUCCACACAAGUGGGCUNN |
| 4997-5015 | 1725 | GUGCUGAAUAGGGAGGAAUNN | 1726 | AUUCCUCCCUAUUCAGCACNN |
| 5094-5112 | 1727 | AGUAAGGGCGUGGAGGCUUNN | 1728 | AAGCCUCCACGCCCUUACUNN |
| 4564-4582 | 1729 | GUGACUUAACCCAAGAAGCNN | 1730 | GCUUCUUGGGUUAAGUCACNN |
| 5095-5113 | 1731 | UAGUAAGGGCGUGGAGGCUNN | 1732 | AGCCUCCACGCCCUUACUANN |
| 4800-4818 | 1733 | UCAUCAGCCUGAUUUUGGUNN | 1734 | ACCAAAAUCAGGCUGAUGANN |
| 4265-4283 | 1735 | GUAGAAGACCCUAAAGACUNN | 1736 | AGUCUUUAGGGUCUUCUACNN |
| 4267-4285 | 1737 | AGGUAGAAGACCCUAAAGANN | 1738 | UCUUUAGGGUCUUCUACCUNN |
| 4270-4288 | 1739 | AAAAGGUAGAAGACCCUAANN | 1740 | UUAGGGUCUUCUACCUUUUNN |
| 4269-4287 | 1741 | AAAGGUAGAAGACCCUAAANN | 1742 | UUUAGGGUCUUCUACCUUUNN |
| 2874-2892 | 1743 | GAUUGUGCAGUGGAAAGAANN | 1744 | UUCUUUCCACUGCACAAUCNN |
| 2875-2893 | 1745 | GGAUUGUGCAGUGGAAAGANN | 1746 | UCUUUCCACUGCACAAUCCNN |
| 3950-3968 | 1747 | UGUAGACAGCCAUAUGCAGNN | 1748 | CUGCAUAUGGCUGUCUACANN |
| 3896-3914 | 1749 | CAUGACUUUAACCCAGAAGNN | 1750 | CUUCUGGGUUAAAGUCAUGNN |
| 4990-5008 | 1751 | AUAGGGAGGAAUCCAUGGANN | 1752 | UCCAUGGAUUCCUCCCUAUNN |
| 4994-5012 | 1753 | CUGAAUAGGGAGGAAUCCANN | 1754 | UGGAUUCCUCCCUAUUCAGNN |
| 5000-5018 | 1755 | AAAGUGCUGAAUAGGGAGGNN | 1756 | CCUCCCUAUUCAGCACUUUNN |
| 4563-4581 | 1757 | UGACUUAACCCAAGAAGCUNN | 1758 | AGCUUCUUGGGUUAAGUCANN |
| 3895-3913 | 1759 | AUGACUUUAACCCAGAAGANN | 1760 | UCUUCUGGGUUAAAGUCAUNN |
| 4262-4280 | 1761 | GAAGACCCUAAAGACUUUCNN | 1762 | GAAAGUCUUUAGGGUCUUCNN |
| 4162-4180 | 1763 | AAAAAGCUCAAAUUUUAUANN | 1764 | UAUAAAAUUUGAGCUUUUUNN |
| 4798-4816 | 1765 | AUCAGCCUGAUUUUGGUACNN | 1766 | GUACCAAAAUCAGGCUGAUNN |
| 4799-4817 | 1767 | CAUCAGCCUGAUUUUGGUANN | 1768 | UACCAAAAUCAGGCUGAUGNN |
| 5006-5024 | 1769 | AUGGACAAAGUGCUGAAUANN | 1770 | UAUUCAGCACUUUGUCCAUNN |
| 4264-4282 | 1771 | UAGAAGACCCUAAAGACUUNN | 1772 | AAGUCUUUAGGGUCUUCUANN |
| 4268-4286 | 1773 | AAGGUAGAAGACCCUAAAGNN | 1774 | CUUUAGGGUCUUCUACCUUNN |
| 4623-4641 | 1775 | CAGCCCACUUGUGUGGAUANN | 1776 | UAUCCACACAAGUGGGCUGNN |
| 4788-4806 | 1777 | UUUUGGUACAUGGAAUAGUNN | 1778 | ACUAUUCCAUGUACCAAAANN |
| 4993-5011 | 1779 | UGAAUAGGGAGGAAUCCAUNN | 1780 | AUGGAUUCCUCCCUAUUCANN |
| 4995-5013 | 1781 | GCUGAAUAGGGAGGAAUCCNN | 1782 | GGAUUCCUCCCUAUUCAGCNN |
| 4996-5014 | 1783 | UGCUGAAUAGGGAGGAAUCNN | 1784 | GAUUCCUCCCUAUUCAGCANN |
| 3952-3970 | 1785 | UGUGUAGACAGCCAUAUGCNN | 1786 | GCAUAUGGCUGUCUACACANN |
| 4595-4613 | 1787 | UGCUUUGAUUGCUUCAGACNN | 1788 | GUCUGAAGCAAUCAAAGCANN |
| 4596-4614 | 1789 | UUGCUUUGAUUGCUUCAGANN | 1790 | UCUGAAGCAAUCAAAGCAANN |
| 4597-4615 | 1791 | AUUGCUUUGAUUGCUUCAGNN | 1792 | CUGAAGCAAUCAAAGCAAUNN |
| 4599-4617 | 1793 | CUAUUGCUUUGAUUGCUUCNN | 1794 | GAAGCAAUCAAAGCAAUAGNN |
| 4726-4744 | 1795 | AUUGCAAGGAAUGGCCUAANN | 1796 | UUAGGCCAUUCCUUGCAAUNN |
| 4753-4771 | 1797 | AUUUUCCUCCUAAUUCUGANN | 1798 | UCAGAAUUAGGAGGAAAAUNN |
| 4802-4820 | 1799 | GCUCAUCAGCCUGAUUUUGNN | 1800 | CAAAAUCAGGCUGAUGAGCNN |
| 4803-4821 | 1801 | UGCUCAUCAGCCUGAUUUUNN | 1802 | AAAAUCAGGCUGAUGAGCANN |
| 4806-4824 | 1803 | AGUUGCUCAUCAGCCUGAUNN | 1804 | AUCAGGCUGAUGAGCAACUNN |
| 5091-5109 | 1805 | AAGGGCGUGGAGGCUUUUUNN | 1806 | AAAAAGCCUCCACGCCCUUNN |
| 5093-5111 | 1807 | GUAAGGGCGUGGAGGCUUUNN | 1808 | AAAGCCUCCACGCCCUUACNN |
| 4259-4277 | 1809 | GACCCUAAAGACUUUCCUGNN | 1810 | CAGGAAAGUCUUUAGGGUCNN |
| 3901-3919 | 1811 | AGGAGCAUGACUUUAACCCNN | 1812 | GGGUUAAAGUCAUGCUCCUNN |
| 4757-4775 | 1813 | UGUGAUUUUCCUCCUAAUUNN | 1814 | AAUUAGGAGGAAAAUCACANN |
| 4758-4776 | 1815 | UUGUGAUUUUCCUCCUAAUNN | 1816 | AUUAGGAGGAAAAUCACAANN |
| 4562-4580 | 1817 | GACUUAACCCAAGAAGCUCNN | 1818 | GAGCUUCUUGGGUUAAGUCNN |
| 4585-4603 | 1819 | GCUUCAGACAAUGGUUUGGNN | 1820 | CCAAACCAUUGUCUGAAGCNN |
| 4587-4605 | 1821 | UUGCUUCAGACAAUGGUUUNN | 1822 | AAACCAUUGUCUGAAGCAANN |
| 4588-4606 | 1823 | AUUGCUUCAGACAAUGGUUNN | 1824 | AACCAUUGUCUGAAGCAAUNN |
| 4591-4609 | 1825 | UUGAUUGCUUCAGACAAUGNN | 1826 | CAUUGUCUGAAGCAAUCAANN |
| 5003-5021 | 1827 | GACAAAGUGCUGAAUAGGGNN | 1828 | CCCUAUUCAGCACUUUGUCNN |
| 4165-4183 | 1829 | AAGAAAAAGCUCAAAUUUUNN | 1830 | AAAAUUUGAGCUUUUUCUUNN |
| 4166-4184 | 1831 | AAAGAAAAAGCUCAAAUUUNN | 1832 | AAAUUUGAGCUUUUUCUUUNN |
| 4263-4281 | 1833 | AGAAGACCCUAAAGACUUUNN | 1834 | AAAGUCUUUAGGGUCUUCUNN |
| 4274-4292 | 1835 | AAAAAAAAGGUAGAAGACCNN | 1836 | GGUCUUCUACCUUUUUUUUNN |
| 4266-4284 | 1837 | GGUAGAAGACCCUAAAGACNN | 1838 | GUCUUUAGGGUCUUCUACCNN |
| 4272-4290 | 1839 | AAAAAAGGUAGAAGACCCUNN | 1840 | AGGGUCUUCUACCUUUUUUNN |
| 4271-4289 | 1841 | AAAAAGGUAGAAGACCCUANN | 1842 | UAGGGUCUUCUACCUUUUUNN |
| 4559-4577 | 1843 | UUAACCCAAGAAGCUCUUCNN | 1844 | GAAGAGCUUCUUGGGUUAANN |
| 4789-4807 | 1845 | AUUUUGGUACAUGGAAUAGNN | 1846 | CUAUUCCAUGUACCAAAAUNN |
| 4998-5016 | 1847 | AGUGCUGAAUAGGGAGGAANN | 1848 | UUCCUCCCUAUUCAGCACUNN |
| 5070-5088 | 1849 | GAGGCCAGGGAAAUUCCCUNN | 1850 | AGGGAAUUUCCCUGGCCUCNN |
| 4158-4176 | 1851 | AGCUCAAAUUUUAUAUAAGNN | 1852 | CUUAUAUAAAAUUUGAGCUNN |
| 5065-5083 | 1853 | CAGGGAAAUUCCCUUGUUUNN | 1854 | AAACAAGGGAAUUUCCCUGNN |
| 2872-2890 | 1855 | UUGUGCAGUGGAAAGAAAGNN | 1856 | CUUUCUUUCCACUGCACAANN |
| 4782-4800 | 1857 | UACAUGGAAUAGUUCAGAGNN | 1858 | CUCUGAACUAUUCCAUGUANN |
| 4783-4801 | 1859 | GUACAUGGAAUAGUUCAGANN | 1860 | UCUGAACUAUUCCAUGUACNN |
| 5064-5082 | 1861 | AGGGAAAUUCCCUUGUUUUNN | 1862 | AAAACAAGGGAAUUUCCCUNN |
| 5071-5089 | 1863 | GGAGGCCAGGGAAAUUCCCNN | 1864 | GGGAAUUUCCCUGGCCUCCNN |
| 3951-3969 | 1865 | GUGUAGACAGCCAUAUGCANN | 1866 | UGCAUAUGGCUGUCUACACNN |
| 3949-3967 | 1867 | GUAGACAGCCAUAUGCAGUNN | 1868 | ACUGCAUAUGGCUGUCUACNN |
| 4355-4373 | 1869 | GAAGACCUGUUUUGCCAUGNN | 1870 | CAUGGCAAAACAGGUCUUCNN |
| 4363-4381 | 1871 | AGUGGGAUGAAGACCUGUUNN | 1872 | AACAGGUCUUCAUCCCACUNN |
| 4356-4374 | 1873 | UGAAGACCUGUUUUGCCAUNN | 1874 | AUGGCAAAACAGGUCUUCANN |
| 4361-4379 | 1875 | UGGGAUGAAGACCUGUUUUNN | 1876 | AAAACAGGUCUUCAUCCCANN |
| 4560-4578 | 1877 | CUUAACCCAAGAAGCUCUUNN | 1878 | AAGAGCUUCUUGGGUUAAGNN |
| 2873-2891 | 1879 | AUUGUGCAGUGGAAAGAAANN | 1880 | UUUCUUUCCACUGCACAAUNN |
| 4730-4748 | 1881 | CUUUAUUGCAAGGAAUGGCNN | 1882 | GCCAUUCCUUGCAAUAAAGNN |
| 3899-3917 | 1883 | GAGCAUGACUUUAACCCAGNN | 1884 | CUGGGUUAAAGUCAUGCUCNN |
| 4756-4774 | 1885 | GUGAUUUUCCUCCUAAUUCNN | 1886 | GAAUUAGGAGGAAAAUCACNN |
| 4590-4608 | 1887 | UGAUUGCUUCAGACAAUGGNN | 1888 | CCAUUGUCUGAAGCAAUCANN |
| 4159-4177 | 1889 | AAGCUCAAAUUUUAUAUAANN | 1890 | UUAUAUAAAAUUUGAGCUUNN |
| 2743-2761 | 1891 | CUGGACAUGGAUCAAGCACNN | 1892 | GUGCUUGAUCCAUGUCCAGNN |
| 4155-4173 | 1893 | UCAAAUUUUAUAUAAGAAANN | 1894 | UUUCUUAUAUAAAAUUUGANN |
| 2871-2889 | 1895 | UGUGCAGUGGAAAGAAAGGNN | 1896 | CCUUUCUUUCCACUGCACANN |
| 4786-4804 | 1897 | UUGGUACAUGGAAUAGUUCNN | 1898 | GAACUAUUCCAUGUACCAANN |
| 4364-4382 | 1899 | AAGUGGGAUGAAGACCUGUNN | 1900 | ACAGGUCUUCAUCCCACUUNN |
| 4359-4377 | 1901 | GGAUGAAGACCUGUUUUGCNN | 1902 | GCAAAACAGGUCUUCAUCCNN |
| 2744-2762 | 1903 | UCUGGACAUGGAUCAAGCANN | 1904 | UGCUUGAUCCAUGUCCAGANN |
| 4787-4805 | 1905 | UUUGGUACAUGGAAUAGUUNN | 1906 | AACUAUUCCAUGUACCAAANN |

TABLE 16
Exemplary JC Virus dsRNAs with NN-dinucleotide overhangs

| Position | SEQ | | SEQ | |
|---|---|---|---|---|
| in | ID | Sequence | ID | Sequence |
| Consensus | NO: | (5′--> 3′) | NO: | (5′--> 3′) |
| 1426-1444 | 1907 | ACUUUUAGGGUUGUACGGGNN | 1908 | CCCGUACAACCCUAAAAGUNN |
| 1427-1445 | 1909 | CUUUUAGGGUUGUACGGGANN | 1910 | UCCCGUACAACCCUAAAAGNN |
| 2026-2044 | 1911 | CAGAGCACAAGGCGUACCUNN | 1912 | AGGUACGCCUUGUGCUCUGNN |
| 1431-1449 | 1913 | UAGGGUUGUACGGGACUGNN | 1914 | ACAGUCCCGUACAACCCUANN |
| 1432-1450 | 1915 | AGGGUUGUACGGGACUGUANN | 1916 | UACAGUCCCGUACAACCCUNN |
| 1436-1454 | 1917 | UUGUACGGGACUGUAACACNN | 1918 | GUGUUACAGUCCCGUACAANN |
| 4794-4812 | 1919 | GCCUGAUUUUGGUACAUGGNN | 1920 | CCAUGUACCAAAAUCAGGCNN |
| 5099-5117 | 1921 | GAAGUAGUAAGGGCGUGGANN | 1922 | UCCACGCCCUUACUACUUCNN |
| 713-731 | 1923 | AUAGGCCUUACUCCUGAAANN | 1924 | UUUCAGGAGUAAGGCCUAUNN |
| 3946-3964 | 1925 | GACAGCCAUAUGCAGUAGUNN | 1926 | ACUACUGCAUAUGGCUGUCNN |
| 1128-1146 | 1927 | AAACUACUUGGGCAAUAGUNN | 1928 | ACUAUUGCCCAAGUAGUUUNN |
| 525-543 | 1929 | UCAGGUUCAUGGGUGCCGCNN | 1930 | GCGGCACCCAUGAACCUGANN |
| 5096-5114 | 1931 | GUAGUAAGGGCGUGGAGGCNN | 1932 | GCCUCCACGCCCUUACUACNN |
| 4727-4745 | 1933 | UAUUGCAAGGAAUGGCCUANN | 1934 | UAGGCCAUUCCUUGCAAUANN |
| 5097-5115 | 1935 | AGUAGUAAGGGCGUGGAGGNN | 1936 | CCUCCACGCCCUUACUACUNN |
| 4601-4619 | 1937 | UGCUAUUGCUUUGAUUGCUNN | 1938 | AGCAAUCAAAGCAAUAGCANN |
| 4600-4618 | 1939 | GCUAUUGCUUUGAUUGCUUNN | 1940 | AAGCAAUCAAAGCAAUAGCNN |
| 1421-1439 | 1941 | CCUUUACUUUUAGGGUUGUNN | 1942 | ACAACCCUAAAAGUAAAGGNN |
| 1424-1442 | 1943 | UUACUUUUAGGGUUGUACGNN | 1944 | CGUACAACCCUAAAAGUAANN |
| 1403-1421 | 1945 | GCUCCUCAAUGGAUGUUGCNN | 1946 | GCAACAUCCAUUGAGGAGCNN |
| 5098-5116 | 1947 | AAGUAGUAAGGGCGUGGAGNN | 1948 | CUCCACGCCCUUACUACUUNN |
| 1430-1448 | 1949 | UUAGGGUUGUACGGGACUGNN | 1950 | CAGUCCCGUACAACCCUAANN |
| 1701-1719 | 1951 | GACAUGCUUCCUUGUUACANN | 1952 | UGUAACAAGGAAGCAUGUCNN |
| 2066-2084 | 1953 | UGUUGAAUGUUGGGUUCCUNN | 1954 | AGGAACCCAACAUUCAACANN |
| 4561-4579 | 1955 | ACUUAACCCAAGAAGCUCUNN | 1956 | AGAGCUUCUUGGGUUAAGUNN |
| 4797-4815 | 1957 | UCAGCCUGAUUUUGGUACANN | 1958 | UGUACCAAAAUCAGGCUGANN |
| 1428-1446 | 1959 | UUUUAGGGUUGUACGGGACNN | 1960 | GUCCCGUACAACCCUAAAANN |
| 1429-1447 | 1961 | UUUAGGGUUGUACGGGACUNN | 1962 | AGUCCCGUACAACCCUAAANN |
| 662-680 | 1963 | UCCCUUGCUACUGUAGAGGNN | 1964 | CCUCUACAGUAGCAAGGGANN |
| 663-681 | 1965 | CCCUUGCUACUGUAGAGGGNN | 1966 | CCCUCUACAGUAGCAAGGGNN |
| 1402-1420 | 1967 | UGCUCCUCAAUGGAUGUUGNN | 1968 | CAACAUCCAUUGAGGAGCANN |
| 1398-1416 | 1969 | GAUCUGCUCCUCAAUGGAUNN | 1970 | AUCCAUUGAGGAGCAGAUCNN |
| 1399-1417 | 1971 | AUCUGCUCCUCAAUGGAUGNN | 1972 | CAUCCAUUGAGGAGCAGAUNN |
| 1400-1418 | 1973 | UCUGCUCCUCAAUGGAUGUNN | 1974 | ACAUCCAUUGAGGAGCAGANN |
| 1401-1419 | 1975 | CUGCUCCUCAAUGGAUGUUNN | 1976 | AACAUCCAUUGAGGAGCAGNN |
| 1435-1453 | 1977 | GUUGUACGGGACUGUAACANN | 1978 | UGUUACAGUCCCGUACAACNN |
| 1437-1455 | 1979 | UGUACGGGACUGUAACACCNN | 1980 | GGUGUUACAGUCCCGUACANN |
| 1438-1456 | 1981 | GUACGGGACUGUAACACCUNN | 1982 | AGGUGUUACAGUCCCGUACNN |
| 4796-4814 | 1983 | CAGCCUGAUUUUGGUACAUNN | 1984 | AUGUACCAAAAUCAGGCUGNN |
| 4992-5010 | 1985 | GAAUAGGGAGGAAUCCAUGNN | 1986 | CAUGGAUUCCUCCCUAUUCNN |
| 4999-5017 | 1987 | AAGUGCUGAAUAGGGAGGANN | 1988 | UCCUCCCUAUUCAGCACUUNN |
| 630-648 | 1989 | AGGCUGCUGCUACUAUAGANN | 1990 | UCUAUAGUAGCAGCAGCCUNN |
| 3947-3965 | 1991 | AGACAGCCAUAUGCAGUAGNN | 1992 | CUACUGCAUAUGGCUGUCUNN |
| 524-542 | 1993 | UUCAGGUUCAUGGGUGCCGNN | 1994 | CGGCACCCAUGAACCUGAANN |
| 3948-3966 | 1995 | UAGACAGCCAUAUGCAGUANN | 1996 | UACUGCAUAUGGCUGUCUANN |
| 3900-3918 | 1997 | GGAGCAUGACUUUAACCCANN | 1998 | UGGGUUAAAGUCAUGCUCCNN |
| 1417-1435 | 1999 | GUUGCCUUUACUUUUAGGGNN | 2000 | CCCUAAAAGUAAAGGCAACNN |
| 4565-4583 | 2001 | UGUGACUUAACCCAAGAAGNN | 2002 | CUUCUUGGGUUAAGUCACANN |
| 4598-4616 | 2003 | UAUUGCUUUGAUUGCUUCANN | 2004 | UGAAGCAAUCAAAGCAAUANN |
| 2060-2078 | 2005 | AUAUCCUGUUGAAUGUUGGNN | 2006 | CCAACAUUCAACAGGAUAUNN |
| 4729-4747 | 2007 | UUUAUUGCAAGGAAUGGCCNN | 2008 | GGCCAUUCCUUGCAAUAAANN |
| 1122-1140 | 2009 | UGGAAGAAACUACUUGGGCNN | 2010 | GCCCAAGUAGUUUCUUCCANN |
| 4261-4279 | 2011 | AAGACCCUAAAGACUUUCCNN | 2012 | GGAAAGUCUUUAGGGUCUUNN |
| 1412-1430 | 2013 | UGGAUGUUGCCUUUACUUUNN | 2014 | AAAGUAAAGGCAACAUCCANN |
| 4592-4610 | 2015 | UUUGAUUGCUUCAGACAAUNN | 2016 | AUUGUCUGAAGCAAUCAAANN |
| 4991-5009 | 2017 | AAUAGGGAGGAAUCCAUGGNN | 2018 | CCAUGGAUUCCUCCCUAUUNN |
| 5004-5022 | 2019 | GGACAAAGUGCUGAAUAGGNN | 2020 | CCUAUUCAGCACUUUGUCCNN |
| 5005-5023 | 2021 | UGGACAAAGUGCUGAAUAGNN | 2022 | CUAUUCAGCACUUUGUCCANN |
| 654-672 | 2023 | AAAUUGCAUCCCUUGCUACNN | 2024 | GUAGCAAGGGAUGCAAUUUNN |
| 659-677 | 2025 | GCAUCCCUUGCUACUGUAGNN | 2026 | CUACAGUAGCAAGGGAUGCNN |
| 4273-4291 | 2027 | AAAAAAAGGUAGAAGACCCNN | 2028 | GGGUCUUCUACCUUUUUUUNN |
| 2025-2043 | 2029 | ACAGAGCACAAGGCGUACCNN | 2030 | GGUACGCCUUGUGCUCUGUNN |
| 4791-4809 | 2031 | UGAUUUUGGUACAUGGAAUNN | 2032 | AUUCCAUGUACCAAAAUCANN |
| 1433-1451 | 2033 | GGGUUGUACGGGACUGUAANN | 2034 | UUACAGUCCCGUACAACCCNN |
| 1434-1452 | 2035 | GGUUGUACGGGACUGUAACNN | 2036 | GUUACAGUCCCGUACAACCNN |
| 1440-1458 | 2037 | ACGGGACUGUAACACCUGCNN | 2038 | GCAGGUGUUACAGUCCCGUNN |
| 1442-1460 | 2039 | GGGACUGUAACACCUGCUCNN | 2040 | GAGCAGGUGUUACAGUCCCNN |
| 1608-1626 | 2041 | ACUCCAGAAAUGGGUGACCNN | 2042 | GGUCACCCAUUUCUGGAGUNN |
| 4793-4811 | 2043 | CCUGAUUUUGGUACAUGGANN | 2044 | UCCAUGUACCAAAAUCAGGNN |

TABLE 17
Sequence of unmodified CNPase dsRNAs

| SEQ | | | |
|---|---|---|---|
| ID | | | |
| NO: | Sense (5′ to 3′) | SEQ ID NO: | Antisense (5′ to 3′) |
| 2057 | GGCCUUGACCUCUUAGAGA | 2058 | UCUCUAAGAGGUCAAGGCC |
| 2059 | GGCCUUGACCUCUUAGAGA | 2060 | UCUCUAAGAGGUCAAGGCC |
| 2061 | GGCCUUGACCUCUUAGAGA | 2062 | UCUCUAAGAGGUCAAGGCC |
| 2063 | GGCCUUGACCUCUUAGAGA | 2064 | UCUCUAAGAGGUCAAGGCC |
| 2065 | GGCCUUGACCUCUUAGAGAUUUU | 2066 | AAAAUCUCUAAGAGGUCAAGGCCUG |
| 2067 | GGCCUUGACCUCUUAGAGA | 2068 | UCUCUAAGAGGUCAAGGCC |
| 2069 | GGGCAAGCUCUAUUCCUUG | 2070 | CAAGGAAUAGAGCUUGCCC |
| 2071 | GCCUUGACCUCUUAGAGAU | 2072 | AUCUCUAAGAGGUCAAGGC |
| 2073 | CCUUGACCUCUUAGAGAUU | 2074 | AAUCUCUAAGAGGUCAAGG |

TABLE 18
Sequences of CNPase NN-dinucleotide modified dsRNAs.

| SEQ | | SEQ | |
|---|---|---|---|
| ID | | ID | |
| NO: | Sense (5′ to 3′) | NO: | Antisense (5′ to 3′) |
| 2075 | GGCCUUGACCUCUUAGAGANN | 2076 | UCUCUAAGAGGUCAAGGCCNN |
| 2077 | GGCCUUGACCUCUUAGAGANN | 2078 | UCUCUAAGAGGUCAAGGCCNN |
| 2079 | GGCCUUGACCUCUUAGAGANN | 2080 | UCUCUAAGAGGUCAAGGCCNN |
| 2081 | GGCCUUGACCUCUUAGAGANN | 2082 | UCUCUAAGAGGUCAAGGCCNN |
| 2083 | GGCCUUGACCUCUUAGAGAUUUUNN | 2084 | AAAAUCUCUAAGAGGUCAAGGCCUGNN |
| 2085 | GGCCUUGACCUCUUAGAGANN | 2086 | UCUCUAAGAGGUCAAGGCCNN |
| 2087 | GGGCAAGCUCUAUUCCUUGNN | 2088 | CAAGGAAUAGAGCUUGCCCNN |
| 2089 | GCCUUGACCUCUUAGAGAUNN | 2090 | AUCUCUAAGAGGUCAAGGCNN |
| 2091 | CCUUGACCUCUUAGAGAUUNN | 2092 | AAUCUCUAAGAGGUCAAGGNN |

TABLE 19
Sequences of modified CNPase dsRNAs.

| | | SEQ | | | SEQ | |
|---|---|---|---|---|---|---|
| Duplex | Strand | ID | | Strand | ID | |
| Name | ID: | NO: | Sense (5′ to 3′) | ID: | NO: | Antisense (5′ to 3′) |
| AD-12436 | AL-20068 | 2096 | GGccuuGAccucuuAGAGATsT | AL-20069 | 2095 | UCUCuAAGAGGUcAAGGCCTsT |
| AD-12449 | AL-20094 | 2098 | GGGcAAGcucuAuuccuuGTsT | AL-20095 | 2097 | cAAGGAAuAGAGCUUGCCCTsT |
| AD-12441 | AL-20078 | 2100 | GccuuGAccucuuAGAGAuTsT | AL-20079 | 2099 | AUCUCuAAGAGGUcAAGGCTsT |
| AD-12438 | AL-20072 | 2102 | ccuuGAccucuuAGAGAuuTsT | AL-20073 | 2101 | AAUCUCuAAGAGGUcAAGGTsT |

Other embodiments are in the claims.

We claim:

1. A method of delivering a double-stranded ribonucleic acid (dsRNA) to a subject, comprising delivering the dsRNA by localized delivery into the corpus callosum of the subject, wherein said dsRNA is one of the dsRNAs selected from Tables 8, 10, and 13-16.

2. The method of claim 1, wherein the dsRNA is delivered to an oligodendrocyte of the subject.

3. The method of claim 1 or 2, wherein the dsRNA comprises at least one modified nucleotide.

4. The method of claim 3, wherein the modified nucleotide is chosen from the group consisting of a 2′-O-methyl modified nucleotide, a nucleotide comprising a 5′-phosphorothioate group, a disulfide linker, and a terminal nucleotide linked to a conjugate group.

5. The method of claim 4, wherein the conjugate group is a cholesteryl derivative or vitamin E group.

6. The method of claim 3, wherein the modified nucleotide is chosen from the group consisting of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

7. A method of inhibiting expression of a gene from JC Virus in a subject, comprising delivering a dsRNA by localized delivery into the corpus callosum of the subject, wherein said dsRNA is selected from the dsRNAs of Tables 8, 10, and 13-16.

8. The method of claim 7, wherein the dsRNA is duplex number AD12795.

9. A method of treating, preventing or managing a pathological process mediated by JC virus in a subject, comprising delivering a dsRNA by localized delivery into the corpus callosum of the subject, wherein said dsRNA is selected from the dsRNAs of Tables 8, 10, and 13-16.

10. A method of delivering a double-stranded ribonucleic acid (dsRNA) to a subject, comprising delivering the dsRNA by localized delivery into the corpus callosum of the subject, wherein said dsRNA is selected from the dsRNAs of Tables 1 and 17-19.

11. The method of claim 9, wherein the dsRNA is delivered to an oligodendrocyte of the subject.

12. The method of claim 9, wherein the dsRNA is AD3222.

13. A method of treating, preventing or managing a neurological disorder mediated by CNPase in a subject, comprising delivering a dsRNA by localized delivery into the corpus callosum of the subject, wherein said dsRNA is selected from the dsRNAs of Tables 1 and 17-19.

14. The method of claim 13, wherein the neurological disorder is schizophrenia.

15. A method of decreasing CNPase mRNA levels in a subject, comprising delivering a dsRNA by localized delivery into the corpus callosum of the subject, wherein the dsRNA is selected from the dsRNAs of Tables 1 and 17-19.

16. The method of claim 15, wherein the dsRNA is AD3222.

17. A method of treating a neurodegenerative disease in a subject comprising delivering a dsRNA by localized delivery into the corpus callosum of the subject.

18. The method of claim 17, wherein the neurodegenerative disease is selected from the group consisting of Parkinson's disease, multiple sclerosis, amyotrophic lateral sclerosis, autoimmune encephalomyelitis, Alzheimer's disease, stroke and Huntington's disease.

19. The method of claim 17 or 18, wherein the dsRNA comprises at least one modified nucleotide.

20. The method of claim 19, wherein the modified nucleotide is chosen from the group consisting of a 2′-O-methyl modified nucleotide, a nucleotide comprising a 5′-phosphorothioate group, a disulfide linker, and a terminal nucleotide linked to a conjugate group.

21. The method of claim 20, wherein the conjugate group is a cholesteryl derivative or vitamin E group.
